(19) United States
(12) Patent Application Publication
(10) Pub. No.: US 2025/0241567 A1
(43) Pub. Date: Jul. 31, 2025

(54) SENSOR SYSTEM FOR OPTICAL DETECTION OF MULTIPLE ANALYTES

(71) Applicant: BlueHalo, LLC, Huntsville, AL (US)

(72) Inventors: Jack T. Ly, Huntsville, AL (US); Kayla F. Presley, Huntsville, AL (US); Matthew J. Dalton, Huntsville, AL (US); Tod A. Grusenmeyer, Huntsville, AL (US)

(21) Appl. No.: 19/076,500

(22) Filed: Mar. 11, 2025

Related U.S. Application Data
(63) Continuation-in-part of application No. 18/590,155, filed on Feb. 28, 2024.
(63) Continuation-in-part of application No. 18/590,178, filed on Feb. 28, 2024.
(60) Provisional application No. 63/487,344, filed on Feb. 28, 2023.
(60) Provisional application No. 63/487,344, filed on Feb. 28, 2023.

(51) Int. Cl. A61B 5/1473; A61B 5/00; A61B 5/145

(52) U.S. Cl. CPC A61B 5/14735; A61B 5/14514; A61B 5/685; A61B 2562/0233; A61B 2562/028

(57) ABSTRACT

A sensor system for optical detection of multiple analytes is discussed. An example system is presented that includes a plurality of lenses configured to receive individual light signals from analyte-sensing chromophores disposed on an array when the analyte-sensing chromophores are illuminated by light while being exposed to different specific analytes within an interstitial fluid. The system further includes at least one volume phase grating (VPG) communicatively coupled to at least one of the plurality of lenses and a detector communicatively coupled to the at least one VPG so that light signals from the analyte-sensing chromophores are focused by the plurality of lenses to the at least one VPG and transmitted to the detector. The detector being operable to generate spectra with each spectrum corresponding to an individual light signal from a single analyte-sensing chromophore.

### CROSS-REFERENCE TO RELATED APPLICATIONS

This application is a continuation in part of U.S. patent application Ser. No. 18/590,155, titled “Optical Microneedle Sensors For Analyte Detection Within Interstitial Fluid,” filed on Feb. 28, 2024, and of U.S. patent application Ser. No. 18/590,178, titled “Optical Microneedle Sensors For Analyte Detection Within Interstitial Fluid,” filed on Feb. 28, 2024, each of which claiming priority to and benefit from U.S. Provisional Patent Application No. 63/487,344, filed on Feb. 28, 2023. The entire disclosure of each of which is hereby incorporated by reference.

### TECHNICAL FIELD

The present disclosure relates generally to a system for monitoring multiple physiological analytes through the use of a single array of optical sensing devices along with an optical detector that can process, transmit, and present results corresponding to such multiple physiological analytes.

### BACKGROUND

Numerous approaches have been developed in order to detect various physiological parameters within a human body. Parameters of interest can include the presence of chemical or biological agents, such as a precursor to, or other indication of, disease, bodily functioning, the administration of medicaments or the like, as part of a health-related diagnostic tool. In situations where these agents are of particular interest in a diagnostic, medical or related analytical procedure, they are referred to as analytes.

One form of conventional analyte sensing employs an approach where injectables that are placed relatively deeply in the skin (such as the lower part of the dermis or even into the subcutaneous layer that forms the hypodermis), often between about 3 to 6 millimeters. This depth is generally too great to permit the use of optical devices operating within the visible light range (that is, penetration depth is too poor for visible light with wavelengths between about 400 and 600 nanometers) and instead necessitates the use of sensors that can only be excited by red or near-infrared (NIR) wavelengths that fall within the optical tissue window (between about 600 and 1300 nanometers). These sensors must also emit light that is within the optical tissue window. Unfortunately, this approach generally requires exotic chromophores in order for the absorption and emission wavelengths to be within this optical tissue window regime. Additionally, an implanted optical sensor may have a limited time of performance, rendering the implanted sensor unusable after a certain amount of time. Moreover, when its usefulness has been exhausted, the sensor (assuming that it is non-biodegradable) is either left permanently implanted in the individual or removed only by an invasive explant procedure. Such implant often requires local tissue healing before use, which, more often than not, requires an equilibration period that limits its immediate efficacy after implantation.

Another form of conventional analyte sensing that is used for monitoring oxygen saturation levels in the body includes pulse oximeters. This approach measures the absorption of blood at two wavelengths (typically in the red or NIR between about 650 and 1000 nanometers) and compares the ratiometric intensity to ascertain oxygenated vs non-oxygenated hemoglobin levels. However, this approach is highly prone to motion artifacts, which can limit its use to static environments and preclude such use for dynamic situations such as those associated with emergency mobile (en route) care and active warfighter environments. The parameter measured in this case (the percent of hemoglobin bound to oxygen) is related to but distinct from direct tissue oxygenation measurements that can occur with a luminescent oxygen sensor. Furthermore, pulse oximetry is not designed to measure hyperoxia—i.e., heightened tissue oxygen concentrations.

In most forms of conventional analyte sensing, each device allows for detection of a single analyte. Although, data on the concentration of multiple analytes would be highly desirable to better understand the health status of an individual, the simultaneous use of multiple devices could be unwieldy and costly.

The foregoing examples of the related art and limitations therewith are intended to be illustrative and not exclusive, and are not admitted to be “prior art.” Other limitations of the related art will become apparent to those of skill in the art upon a reading of the specification and a study of the drawings.

### SUMMARY

The system disclosed herein is directed to an optical sensing device capable of detecting multiple physiological analytes in a continuous manner, the optical sensing device being signally coupled to a detector-based system that enables continuous, real-time monitoring of such analytes. In some embodiments, one or both of the optical sensing device and detector-based system define a wearable form factor. Through the simultaneous acquisition, processing and transmission of data from numerous different analytes, the amount of health information gained from a single sensing device array and a single system, such as those disclosed herein, can be equivalent to the amount of health-related metrics that typically require unwieldly, complex systems to acquire.

In some embodiments, the optical sensing device is made up of a microneedle array. In particular, such microneedles extend only within a relatively short distance (for example, between 0.4 and 1.5 millimeters from base to tip) into a patient's skin such that unlike hypodermic needles or related invasive mechanisms, they do not activate the patient's dermal nerve network, thereby allowing the intended analysis to occur in a relatively pain-free, minimally invasive manner. In addition, because the interstitial fluid within tissue spaces in the skin is very similar to blood plasma in terms of concentration of particular analytes (at least for those analytes that are small molecules), a microneedle array may be used to reach the interstitial fluid without having to go through hypodermic or related subcutaneous means. Furthermore, by functionalizing the microneedle array with analyte-sensing chromophores capable of detecting multiple different analytes, the disclosed assembly can function as an optically transparent transdermal window so that light signals being introduced thereto may be absorbed by the chromophore. Because each chromophore's optical response may be altered by the presence of a particular analyte (for example, dissolved oxygen), optical responses can include changes in resulting emission intensity, changes in resulting emission lifetime, and observed color changes (also known as colorimetry).

According to some embodiments, by coupling the single array of optical sensing devices to suitable optical detection and associated electronic processing equipment, the acquired signals can be used as part of an intelligible analysis to detect various analytes within the interstitial fluid of an individual, including situations where numerous analytes are being detected simultaneously. In some embodiments where the optical sensing devices are microneedles or related optical-based components, conventional electrode-based electrochemical approaches requiring conversion of a reaction into a suitable voltage, current, resistance or related electrical signal are avoided, as are fluid collection reservoirs. Moreover, the systems and methods disclosed herein may be used in dynamic (that is to say, motion-related) environments in a manner that may otherwise hamper the collection and analysis of data under some conventional approaches. In some embodiments, the microneedles are formed as solid or hollow structures, and in either case are configured to include analyte-sensing chromophores such that luminescence changes occur in the presence of an analyte. As noted, different microneedles within the array may be tailored to different analyte-sensing chromophores such that luminescence changes that occur to each of these chromophores in the presence of one or more analytes can-when coupled to software that makes up a part of the optical detector and associated parts of the system-identify the presence of the corresponding analytes.

According to an aspect of the present disclosure, an analyte-sensing system is disclosed. The system includes a microneedle assembly with a substrate, one or more microneedles disposed on a skin-facing surface of the substrate such that upon placement of the skin-facing surface on the skin of an individual, the at least one microneedle extends a distance sufficient into one or more layers of the skin such that it is immersed in an interstitial fluid, and a biocompatible polymer cooperative with the one or more microneedles. The one or more microneedles are optically transparent to form an optically transparent transdermal window, while the biocompatible polymer includes an analyte-sensing chromophore and is configured such that at least a portion of an analyte present in the interstitial fluid diffuses into the biocompatible polymer. In this way, upon the introduction of a light signal to the transdermal window, the presence of the diffused analyte in the biocompatible polymer causes a change to at least one photophysical property of the analyte-sensing chromophore that can be optically detected and converted into indicia of the analyte that is present in the interstitial fluid.

According to another aspect of the present disclosure, a method of transdermally analyzing an interstitial fluid is disclosed. The method includes receiving a light signal into a transdermal window that is placed into numerous layers of the skin of an individual so that upon having at least a portion of an analyte present in the interstitial fluid diffuse into a portion of the transdermal window, an analyte-sensing chromophore that is part of a biocompatible polymer undergoes a change in at least one photophysical property in response to an interaction between the diffused analyte and the analyte-sensing chromophore. The method further includes sensing, with a reader in optical communication with the transdermal window, at least a portion of the received light signal that contains indicia of the change to the at least one photophysical property.

According to another aspect of the present disclosure, an analyte-sensing system is disclosed. The analyte-sensing system includes an optical sensor array comprising numerous microneedle-chromophore combinations that extend a distance sufficient into a layer of an individual's skin such that they are immersed in an interstitial fluid of the individual's skin. Each of the microneedle-chromophore combinations detects an analyte that may be a different analyte than at least one other of the microneedle-chromophore combinations through an agent that cooperates with a respective one of the optical microneedles to form a transdermal window. The analyte-sensing system further includes one or more collimating lenses and a coupling device disposed between the optical sensor array and the one or more collimating lenses to convey between them at least some of the optical signal from the chromophores. One or more volume phase gratings are used to form a spectrum for each of the optical signals that pass through the one or more collimating lenses. One or more focusing lenses project these optical signals onto a detector such that upon the introduction of a light signal to each microneedle-chromophore combination, the light signal interacts with at least a portion of a particular analyte that is present in the interstitial fluid and that has diffused into the transdermal window to cause a change to at least one of the optically-detectable analyte-sensing chromophore's photophysical properties, after which a spectrum for each of the plurality of optically-detectable analyte-sensing chromophores passes through one or more lenses and volume phase gratings such that the signal may be registered on the detector as a data cube.

According to another aspect of the present disclosure, an optical processing system is disclosed. The optical processing system includes one or more lenses configured to receive numerous individual light signals, one or more volume phase gratings cooperative with the one or more lenses to form a spectrum for each of the various signals, one or more focusing lenses, and a detector cooperative with these other components. In this way, upon the introduction of each of the individual light signals to the lens, the signals pass through the lens, volume phase grating and into the detector that in turn may generate a data cube within the detector. The foregoing Summary, including the description of some embodiments, motivations therefor, and/or advantages thereof, is intended to assist the reader in understanding the present disclosure, and does not in any way limit the scope of any of the claims.

### BRIEF DESCRIPTION OF THE DRAWINGS

The accompanying figures (FIGS), which are included as part of the present specification, illustrate the presently preferred embodiments and together with the general description given above and the detailed description of the preferred embodiments given below serve to explain and teach the principles described herein. In the figures, like structures are indicated with like reference numerals.

FIGS. 1A and 1B depict a computer-based analyte-sensing system with a skin-mounted optical microneedle array, in accordance to some embodiments.

FIG. 2 depicts the optical microneedle array of FIGS. 1A and 1B where a multiplex array of different individual microneedles is shown, each containing a tailored analyte-sensing chromophore, in accordance to some embodiments.

FIG. 3 depicts a Pd(II) meso-tetra(N-(3-methacrylamidopropyl)benzamido) porphyrin molecule that forms an analyte-sensing chromophore, in accordance to some embodiments.

FIG. 4 depicts a Pd(II) meso-tetra(4-carboxyphenyl) porphyrin molecule that forms a analyte-sensing chromophore, in accordance to some embodiments.

FIG. 5A depicts a phosphorescence lifetime modulation for a Pd(II) meso-tetra(N-(3-methacrylamidopropyl)benzamido) porphyrin-loaded hydrogel coating placed on a microneedle array in 37° C. phosphate-buffered saline over varying concentrations of dissolved oxygen, in accordance to some embodiments.

FIG. 5B depicts a Stern-Volmer plot of the Pd(II) meso-tetra(N-(3-methacrylamidopropyl)benzamido) porphyrin-loaded hydrogel coating of FIG. 5A in 37° C. phosphate-buffered saline, in accordance to some embodiments.

FIG. 6 depicts a mechanism for sodium ion-induced fluorescence emission enhancement in an aza-crown ether functionalized boron-dipyrromethene (BODIPY)-based analyte-sensing chromophore, in accordance to some embodiments.

FIGS. 7A, 7B, and 7C depict various views of a microneedle application device and its use in applying it to the skin of an individual as part of a transdermal analysis of an interstitial fluid, in accordance to some embodiments.

FIG. 8 depicts the use of inkjet printing for performing microneedle coating, in accordance to some embodiments.

FIG. 9 depicts an example of an array patterning technique implemented through inkjet printing, in accordance to some embodiments.

FIG. 10 depicts chromophore-loaded hydrogel-coated microneedles made from airbrush coating with a laser cut mask of 390×390 micrometer square openings, in accordance to some embodiments.

FIG. 11 depicts magnified optical and fluorescence confocal images of hydrogel-coated microneedles, in accordance to some embodiments.

FIGS. 12A and 12B depict synthesis approaches for chromophores capable of sensing sodium ions, in accordance to some embodiments.

FIGS. 13A and 13B depicts synthesis approaches for chromophores capable of sensing potassium ions, in accordance to some embodiments.

FIG. 14 depicts synthesis approaches for chromophores capable of sensing calcium ions, in accordance to some embodiments.

FIG. 15 depicts an example of a synthesis pathway for a crosslinkable chromophore for use in sodium ion sensing;

FIGS. 16A through 16B depicts synthesis pathways to afford methacrylate functionality via para functionalization of diphenylpyrrole BODIPY precursors, in accordance to some embodiments.

FIG. 17 depicts examples of crosslinkable, synthesizable chromophores with various sensing moieties for sodium, potassium and calcium ions, in accordance to some embodiments.

FIG. 18 depicts examples of non-crosslinkable, synthesizable chromophores with various sensing moieties for sodium, potassium and calcium ions, in accordance to some embodiments.

FIG. 19 depicts examples of crosslinkable, synthesizable quinolinium- and acridinium-based chromophores for chloride ion sensing, in accordance to some embodiments.

FIG. 20 depicts examples of crosslinkable, synthesizable cyclometallated-iridium(III) BODIPY chromophores for ratiometric dissolved oxygen sensing and crosslinkable, synthesizable N-phenyl-boronic acid BODIPYs for lactate sensing, in accordance to some embodiments.

FIG. 21 depicts examples of non-crosslinkable, synthesizable cyclometallated-iridium (III) BODIPY chromophores for ratiometric dissolved oxygen sensing and non-crosslinkable, synthesizable N-phenyl-boronic acid BODIPYs for lactate sensing, in accordance to some embodiments.

FIG. 22 depicts examples of crosslinkable, synthesizable iodo-BODIPY chromophores for ratiometric dissolved oxygen sensing, in accordance to some embodiments.

FIG. 23 depicts an analyte-sensing system that uses an optical sensor array for simultaneous detection of multiple analytes in real-time, in accordance to some embodiments.

FIG. 24 depicts a variation of the analyte-sensing system of FIG. 23 where a microelectromechanical system is used to facilitate sequential detection of multiple analytes, in accordance to some embodiments.

FIG. 25 depicts a waveguide of FIG. 24 as a tapered fused fiber waveguide, in accordance to some embodiments.

FIG. 26 depicts a waveguide of FIG. 24 as a semiconductor photonic waveguide, in accordance to some embodiments.

FIG. 27 depicts another variation of the analyte-sensing system of FIG. 23 where line scanning is used rather than a one-time snapshot of the entire optical sensor array, in accordance to some embodiments.

While the present disclosure is subject to various modifications and alternative forms, specific embodiments thereof have been shown by way of example in the drawings and will herein be described in detail. The present disclosure should not be understood to be limited to the particular forms disclosed, but on the contrary, the intention is to cover all modifications, equivalents, and alternatives falling within the spirit and scope of the present disclosure.

### DETAILED DESCRIPTION

A key challenge in conventional optical sensors is how to provide minimally invasive biosampling of physiological fluids, as well as various biochemicals, metabolites, electrolytes, ions, pathogens, microorganisms or other analytes present within such fluids. One part of this difficulty relates to how to monitor for one or more indicators of bodily condition, including (among others) hypoxia, hyperoxia, hydration, stress and fatigue through the detection of various analytes within physiological fluids. Yet another challenge is that conventional implantable optical sensors must operate in the optical tissue window (which generally exists at between 600 and 1300 nanometers (nm)); such range precludes the use of many chromophores. Still another challenge is that for colorimetric measurements, optical constraints and other factors would be hard to implement in implantable sensors. A further challenge for implantable sensors arises for intensity-based measurements, specifically because such measurements require considerable correction factors. For instance, even with a ratiometric sensor, the sensor and reference wavelengths would exhibit different degrees of scattering and absorption traveling from the implant to the reader. Another challenge with electrochemical-based devices is that certain parts related to the electronics must remain implanted into or attached to the body, even when the sensor is not in use. Lastly, if analyte detection were done via the method of an assay after a blood draw or related procedure, it would not be possible to achieve real-time, continuous monitoring.

The technical solution presented herein is to provide one or more optical microneedles (also referred to herein as optically transparent microneedles) that permit the passage of a light wave that is within the visible spectrum through a microneedle such that upon interaction of the light with a portion of the physiological fluid that has interacted with a hydrogel or related biocompatible polymer that includes an analyte-sensing chromophore and that makes up a part of the microneedle, a corresponding signal may be analyzed to produce indicia of conditions within the physiological fluid. As will be discussed in more detail, the use of a transdermal window with one or more optically transparent microneedles allows for the use of chromophores that can function outside of the optical tissue window—in other words, chromophores that can be excited by and/or can both be excited by and emit wavelengths less than 600 nm (<600 nm). In some cases, this could allow for the use of commercially available chromophores, and when this is not possible, difficulties associated with the synthesis of non-commercially available chromophores are substantially obviated without having to operate in the optical tissue window. Thus, upon excitation by light of a known wavelength, a detectable change in at least one photophysical property of the chromophore (such as a change in the emission lifetime or intensity) that occurs in the presence of the analyte takes place; this change can be detected and quantified.

During use, the hydrogel or related polymer that forms a part of the microneedles becomes illuminated with light in a first wavelength range. According to some embodiments, the microneedles are sized, for example, to allow roughly 1 millimeter-deep skin penetration to interface with the interstitial fluid that is present within the dermis layer of the skin and which may include one or more of sugars, salts, fatty acids, electrolytes, dissolved oxygen, amino acids, co-enzymes, hormones, neurotransmitters, white blood cells and/or the like. The shallow penetration depth of the microneedles (e.g., about 1 millimeter (mm)) may not require a long equilibration period and thus it may be possible to monitor for the presence of a particular analyte relatively soon after placement and penetration of the skin surface. In general, upon receipt within the biocompatible polymer of at least a portion of the light in this first wavelength range, the analyte-sensing chromophore either absorbs some portion of the light and/or is excited by this light and emits a second (that is to say, longer) wavelength range. Interaction of the analyte-sensing chromophore and the analyte results in a change in at least one photophysical property of the chromophore (that is to say, changes in luminescence emission intensity, phosphorescence emission lifetime, absorption maxima, emission maxima, absorption (resulting in color change) or the like). By way of example, in the case of dissolved oxygen sensing, a phosphorescent chromophore is excited and undergoes intersystem crossing to its triplet state. The phosphorescence emission is quenched in accordance with the dissolved oxygen concentration such that both the phosphorescence lifetime and emission intensity are reduced in the presence of dissolved oxygen. In one instance, dissolved oxygen could be sensed by monitoring changes in the lifetime of a phosphorescent chromophore. Dissolved oxygen could also be sensed using a ratiometric intensity approach through the use of a dual luminescent chromophore that exhibits both an oxygen-insensitive fluorescence emission and an oxygen-sensitive phosphorescence emission. In this case, the ratio between the two emission intensities could be used to indicate the amount of dissolved oxygen present. By way of example, glucose or lactate could also be sensed through use of an oxygen-sensitive chromophore and either glucose oxidase or lactate oxidase, respectively. In this manner, dissolved oxygen is consumed in the presence of the analyte (glucose or lactate) and its respective enzyme (glucose oxidase or lactate oxidase, respectively). This change in local oxygen consumption would result in reduced quenching of the phosphorescence of the oxygen-sensitive chromophore, and the changes in the photophysical properties of the oxygen-sensitive chromophore could be used to indirectly assess the glucose or lactate concentration. By way of example, glucose and lactate could also be sensed via the use of an appropriate boronic acid attached to the chromophore. In some embodiments, the boronic acid should be chosen to selectively bind glucose or lactate, and the complexation of the boronic acid and the analyte could result in changes to the emission intensity of the chromophore. By way of example, in the case of electrolyte sensing, a luminescent molecule can be designed to exhibit a change in one or more photophysical properties as the concentration of the particular electrolyte of interest increases. More specifically, the use of an appropriately sized crown ether attached to a luminescent molecule could be used to selectively bind sodium or potassium ions in such a manner that emission intensity increases in the presence of the analyte. For sensing of chloride ions, the luminescent emission of certain quinolinium chromophores, quinolinium-containing chromophores, acridinium chromophores and acridinium-containing chromophores can be quenched by chloride ions. For detection of calcium ions, a luminescent chromophore can be made to contain chelating agents, such as 1,2-bis(o-aminophenoxy) ethane-N,N,N′,N′-tetra acetic acid (BAPTA) or N-phenylazabis(ethoxy)acetic acid, that selectively bind calcium ions. In the presence of calcium, the luminescent chromophore may exhibit an increase in emission intensity, a change in the absorption maximum, and/or a change in the emission maximum. By way of example, for a colorimetric pH sensor, changes in absorption due to changes in the pH of the interstitial fluid result in color changes of the chromophore. By way of example, for detection of larger molecules, such as hormones, neuropeptides, and neurotransmitters, the use of a biorecognition element is likely needed to impart selectivity. For instance, a deoxyribonucleic acid (DNA) or ribonucleic acid (RNA) aptamer could serve as the biorecognition element and could be paired with a luminescent chromophore in such a way that changes in at least one photophysical property could be observed in the presence of the analyte. Luminescent aptamer-based strategies could encompass mechanisms based on FRET (Förster or fluorescence resonance energy transfer). By way of example, one end of an aptamer may be bound to the luminescent chromophore while the other end may be bound to a quencher. In the absence of the analyte, the chromophore emission could be at least partially quenched, but binding of the analyte to the aptamer could separate the luminescent chromophore and quencher from one another, thereby increasing emission intensity. The aptamer in this case could be a hairpin aptamer. Another instance of a luminescent aptamer-based strategy is a means by which the binding of the analyte changes the environment of the chromophore in such a manner as to alter at least one of its photophysical properties. By way of example, this could entail the use of a conformation-dependent luminescent chromophore along with a forced intercalation (FIT) aptamer.

Moreover, configuring the microneedles to have relatively short skin penetration depths into the region of interest in conjunction with optically transparent construction permits the operating light range to also take place at wavelengths below 600 nm (<600 nm) rather than being restricted to the optical tissue window (e.g., between about 600 and 1300 nm). This in turn allows the functional biocompatible polymer sensor formulation to expand the analyte-sensing chromophore to a much wider variety of options. These options include commercially available chromophores, chromophores that can be commercially purchased and then modified in some way (i.e., to add functionality), and synthesizable chromophores. Chromophores that may be commercially available and could be used include Pd(II) meso-tetra(4-carboxyphenyl) porphyrin which could be used for the sensing of dissolved oxygen. This chromophore could also be used for sensing of lactate or glucose in the presence of lactate oxidase or glucose oxidase, respectively. In addition, commercially available analyte-sensing chromophores that may be employed for pH sensing include bromothymol blue, phenol red, cresol red, neutral red, α-naphtholphthalein and m-cresol purple. Commercially available chromophores for sodium ion sensing could include CoroNa Green and Sodium Green, while potassium benzofuran isophthalic acid is a commercially available chromophore that could enable potassium ion sensing. Oregon Green 488 BAPTA-5N hexapotassium salt, Fluo-4FF pentapotassium salt, and Fura Red acetoxymethyl could be commercially available chromophores for calcium ion sensing, while commercially available chromophores for chloride ion sensing could include Lucigenin (10,10′-dimethyl-9,9′-biacridinium, dinitrate), 6-methoxy-N-(3-sulfopropyl) quinolinium and N-(ethoxycarbonylmethyl)-6-methoxyquinolinium bromide. It will be appreciated that these commercially available chromophores and target analytes are mentioned as non-limiting examples, and that other target analytes and suitably-configured commercially available chromophores for use with the biocompatible polymer are deemed to be within the scope of the present disclosure. In some embodiments, commercially available chromophores may be customized to impart desirable functionalities. By way of example and not limitation, a moiety can be added to a chromophore to impart the chromophore with analyte-sensing capabilities. Another instance of this is adding functionality that enables covalent attachment of the analyte-sensing chromophore to the biocompatible polymer. By way of example, Pd(II) meso-tetra(N-(3-methacrylamidopropyl)benzamido) can be created via the addition of methylmethacrylate functional groups to the commercially available Pd(II) meso-tetra(4-carboxyphenyl) porphyrin, and this added functionality can enable crosslinking to an acrylate-based polymer. It will be appreciated that Pd(II) meso-tetra(N-(3-methacrylamidopropyl)benzamido) is mentioned as a non-limiting example and that other modified chromophores for use with the biocompatible polymer are deemed to be within the scope of the present disclosure. As aforementioned, the analyte-sensing chromophore may instead need to be synthesized. Synthesizable dual luminescent chromophores that could be used for ratiometric intensity based detection of dissolved oxygen and could be crosslinked to acrylate-based polymers include iodo-BODIPYs, such as 2,6-diiodo-3,5-distyryl BODIPY and 2,6-diiodo-3-styryl BODIPY, and cyclometallated-iridium (III) BODIPY complexes, such as [(iridium(III) biphenylisoquinoline isocyano(dimethylbenzo) pyridine) (2,6-diphenylmethacrylate BODIPY)](hexafluorophosphate), [(iridium(III) biphenylisoquinoline isocyano(dimethylbenzo) pyridine) (2,6-diphenylethyl methacrylate BODIPY)](hexafluorophosphate), [(iridium(III) biphenylisoquinoline isocyano(dimethylbenzo) pyridine) (2,6-divinyl BODIPY)](hexafluorophosphate), [(iridium(III) biphenylisoquinoline isocyano(dimethylbenzo) pyridine) (2,6-diallyl BODIPY)](hexafluorophosphate), [(iridium(III) biphenylisoquinoline isocyano(dimethylbenzo) pyridine) (2,6-dibutenyl BODIPY)](hexafluorophosphate), [(iridium(III) biphenylisoquinoline isocyano(dimethylbenzo) pyridine) (3,5-distyryl BODIPY)](hexafluorophosphate), and [(iridium(III) biphenylisoquinoline isocyano(dimethylbenzo) pyridine) (3-styryl BODIPY)](hexafluorophosphate)]. Synthesizable chromophores that do not have the functionality to enable crosslinking to acrylate-based polymers but enable sensing of dissolved oxygen include tetra-substituted [(iridium(III) biphenylisoquinoline isocyano(dimethylbenzo) pyridine) (1,3,5,7-tetraphenyl BODIPY)](hexafluorophosphate). Synthesizable sodium ion sensing chromophores that can be crosslinked to acrylate-based polymers include N-phenyl-aza-15-crown-5-ether 2,6-diphenylmethacrylate BODIPY, N-phenyl-aza-15-crown-5-ether 2,6-diphenylethyl methacrylate BODIPY, N-phenyl-aza-15-crown-5-ether 2,6-divinyl BODIPY, N-phenyl-aza-15-crown-5-ether 2,6-diallyl BODIPY, N-phenyl-aza-15-crown-5-ether 2,6-dibutenyl BODIPY, N-phenyl-aza-15-crown-5-ether 3,5-distyryl BODIPY, N-phenyl-aza-15-crown-5-ether 3-styryl BODIPY, o-methoxy-N-phenyl-aza-15-crown-5-ether 2,6-diphenylmethacrylate BODIPY, o-methoxy-N-phenyl-aza-15-crown-5-ether 2,6-diphenylethyl methacrylate BODIPY, o-methoxy-N-phenyl-aza-15-crown-5-ether 2,6-divinyl BODIPY, o-methoxy-N-phenyl-aza-15-crown-5-ether 2,6-diallyl BODIPY, o-methoxy-N-phenyl-aza-15-crown-5-ether 2,6-dibutenyl BODIPY, o-methoxy-N-phenyl-aza-15-crown-5-ether 3,5-distyryl BODIPY, o-methoxy-N-phenyl-aza-15-crown-5-ether 3-styryl BODIPY, o-methoxyethoxy-N-phenyl-aza-15-crown-5-ether 2,6-diphenylmethacrylate BODIPY, o-methoxyethoxy-N-phenyl-aza-15-crown-5-ether 2,6-diphenylethyl methacrylate BODIPY, o-methoxyethoxy-N-phenyl-aza-15-crown-5-ether 2,6-divinyl BODIPY, o-methoxyethoxy-N-phenyl-aza-15-crown-5-ether 2,6-diallyl BODIPY, o-methoxyethoxy-N-phenyl-aza-15-crown-5-ether 2,6-dibutenyl BODIPY o-methoxyethoxy-N-phenyl-aza-15-crown-5-ether 3,5-distyryl BODIPY and o-methoxyethoxy-N-phenyl-aza-15-crown-5-ether 3-styryl BODIPY. Furthermore, synthesizable potassium ion sensing chromophores that can be crosslinked to acrylate-based polymers include N-phenyl-aza-18-crown-6-ether 2,6-diphenylmethacrylate BODIPY, N-phenyl-aza-18-crown-6-ether 2,6-diphenylethyl methacrylate BODIPY, N-phenyl-aza-18-crown-6-ether 2,6-divinyl BODIPY, N-phenyl-aza-18-crown-6-ether 2,6-diallyl BODIPY, N-phenyl-aza-18-crown-6-ether 2,6-dibutenyl BODIPY, N-phenyl-aza-18-crown-6-ether 3,5-distyryl BODIPY, N-phenyl-aza-18-crown-6-ether 3-styryl BODIPY, o-methoxy-N-phenyl-aza-18-crown-6-ether 2,6-diphenylethyl methacrylate BODIPY, o-methoxy-N-phenyl-aza-18-crown-6-ether 2,6-divinyl BODIPY, o-methoxy-N-phenyl-aza-18-crown-6-ether 2,6-diallyl BODIPY, o-methoxy-N-phenyl-aza-18-crown-6-ether 2,6-dibutenyl BODIPY, o-methoxy-N-phenyl-aza-18-crown-6-ether 3,5-distyryl BODIPY, o-methoxy-N-phenyl-aza-18-crown-6-ether 3-styryl BODIPY, o-methoxyethoxy-N-phenyl-aza-18-crown-6-ether 2,6-diphenylethyl methacrylate BODIPY, o-methoxyethoxy-N-phenyl-aza-18-crown-6-ether 2,6-divinyl BODIPY, o-methoxyethoxy-N-phenyl-aza-18-crown-6-ether 2,6-diallyl BODIPY, o-methoxyethoxy-N-phenyl-aza-18-crown-6-ether 2,6-dibutenyl BODIPY, o-methoxyethoxy-N-phenyl-aza-18-crown-6-ether 3,5-distyryl BODIPY and o-methoxyethoxy-N-phenyl-aza-18-crown-6-ether 3-styryl BODIPY. Synthesizable calcium ion sensing chromophores that can be crosslinked to acrylate-based polymers includes N-phenylazabis(ethoxy)acetic acid 2,6-diphenylmethacrylate BODIPY, N-phenylazabis(ethoxy)acetic acid 2,6-diphenylethyl methacrylate BODIPY, N-phenylazabis(ethoxy)acetic acid 2,6-divinyl BODIPY, N-phenylazabis(ethoxy)acetic acid 2,6-diallyl BODIPY, N-phenylazabis(ethoxy)acetic acid 2,6-dibutenyl BODIPY, N-phenylazabis(ethoxy)acetic acid moieties 3,5-distyryl BODIPY, N-phenylazabis(ethoxy)acetic acid moieties 3-styryl BODIPY, o-methoxy-N-phenylazabis(ethoxy)acetic acid 2,6-diphenylmethacrylate BODIPY, o-methoxy-N-phenylazabis(ethoxy)acetic acid 2,6-diphenylethyl methacrylate BODIPY, o-methoxy-N-phenylazabis(ethoxy)acetic acid 2,6-divinyl BODIPY, o-methoxy-N-phenylazabis(ethoxy)acetic acid 2,6-diallyl BODIPY, o-methoxy-N-phenylazabis(ethoxy)acetic acid 2,6-dibutenyl BODIPY, o-methoxy-N-phenylazabis(ethoxy)acetic acid moieties 3,5-distyryl BODIPY, o-methoxy-N-phenylazabis(ethoxy)acetic acid moieties 3-styryl BODIPY, o-methoxyethoxy-N-phenylazabis(ethoxy)acetic acid 2,6-diphenylmethacrylate BODIPY, o-methoxyethoxy-N-phenylazabis(ethoxy)acetic acid 2,6-diphenylethyl methacrylate BODIPY, o-methoxyethoxy-N-phenylazabis(ethoxy)acetic acid 2,6-divinyl BODIPY, o-methoxyethoxy-N-phenylazabis(ethoxy)acetic acid 2,6-diallyl BODIPY, o-methoxyethoxy-N-phenylazabis(ethoxy)acetic acid 2,6-dibutenyl BODIPY, o-methoxyethoxy-N-phenylazabis(ethoxy)acetic acid moieties 3,5-distyryl BODIPY and o-methoxyethoxy-N-phenylazabis(ethoxy)acetic acid moieties 3-styryl BODIPY. Synthesizable chromophores that do not have the functionality to enable crosslinking to acrylate-based polymers include N-phenyl-aza-15-crown-5-ether BODIPY, tetra-substituted N-phenyl-aza-15-crown-5-ether 1,3,5,7-tetraphenyl BODIPY, tetra-substituted o-methoxy-N-phenyl-aza-15-crown-5-ether 1,3,5,7-tetraphenyl BODIPY, and tetra-substituted o-methoxyethoxy-N-phenyl-aza-15-crown-5-ether 1,3,5,7-tetraphenyl BODIPY which could be used to sense sodium ions, N-phenyl-aza-18-crown-6-ether BODIPY, tetra-substituted N-phenyl-aza-18-crown-6-ether 1,3,5,7-tetraphenyl BODIPY, tetra-substituted o-methoxy-N-phenyl-aza-18-crown-6-ether 1,3,5,7-tetraphenyl BODIPY, and tetra-substituted o-methoxyethoxy-N-phenyl-aza-18-crown-6-ether 1,3,5,7-tetraphenyl BODIPY which could be used to sense potassium ions, and N-phenylazabis(ethoxy)acetic acid BODIPY, tetra-substituted N-phenylazabis(ethoxy)acetic acid 1,3,5,7-tetraphenyl BODIPY, tetra-substituted o-methoxy-N-phenylazabis(ethoxy)acetic acid 1,3,5,7-tetraphenyl BODIPY, and tetra-substituted o-methoxyethoxy-N-phenylazabis(ethoxy)acetic acid 1,3,5,7-tetraphenyl BODIPY which could be used to sense calcium ions. Synthesizable chromophores that may be able to be used to sense chloride ions and could be crosslinked to acrylate-based polymers include ethyl-2-(quinolinium methyl) methacrylate bromide, 2-quinolinium ethyl methacrylate bromide, ethyl-2-(acridinium methyl) methacrylate bromide, 2-acridinium ethyl methacrylate bromide, 9,9′-bi (ethyl-2-(acridinium methyl) methacrylate bromide), and 9,9′-bi (2-acridinium ethyl methacrylate bromide). Synthesizable chromophores that could be used to detect lactate via intensity-based detection and could be crosslinked to acrylate-based polymers include N-phenyl-boronic acid 2,6-diphenylmethacrylate BODIPY, N-phenyl-boronic acid 2,6-diphenylethyl methacrylate BODIPY, N-phenyl-boronic acid 2,6-divinyl BODIPY, N-phenyl-boronic acid 2,6-diallyl BODIPY, N-phenyl-boronic acid 2,6-dibutenyl BODIPY, N-phenyl-boronic acid 3,5-distyryl BODIPY, and N-phenyl-boronic acid 3-styryl BODIPY. Synthesizable chromophores that do not have the functionality to enable crosslinking to acrylate-based polymers but enable sensing of lactate include tetra-substituted N-phenyl-boronic acid 1,3,5,7-tetraphenyl BODIPY. It will be appreciated that although such chromophores may not be commercially available, those skilled in the art may readily convert them through known synthetic approaches. It will be appreciated that these synthesizable chromophores and target analytes are mentioned as non-limiting examples, and that other target analytes and suitably-configured synthesizable chromophores for use with the biocompatible polymer are deemed to be within the scope of the present disclosure. It will be appreciated that the foregoing list is not all-inclusive, and that additional chromophores such as those that are depicted in the various figures, as well as those discussed elsewhere herein, may be included in the foregoing list, and that all such chromophores and their variants are within the scope of the present disclosure. For example, some of the chromophores depicted in the various figures are described through the use of R groups; it will be appreciated that such shorthand notation encompasses other possible structures all of which are within the scope of the present disclosure. It will be further appreciated that while some of these structures have been previously demonstrated to show analyte-sensing capabilities, they can be incorporated into polymer-coated microneedles as described herein for continuous monitoring within the interstitial fluid (ISF), especially when used as part of a targeted formulation. By way of example, N-phenyl-aza-18-crown-6-ether BODIPY and N-phenylazabis(ethoxy)acetic acid BODIPY are known in the literature for in vitro potassium ion (K⁺) and calcium ion (Ca²⁺) sensing, respectively.

According to some embodiments, the use of optically transparent microneedles in conjunction with biocompatible polymers (such as hydrogels, whether formed as a coating or in situ) makes it possible to sense analytes in one of three analytical ways: (i) a lifetime approach, (ii) an intensity approach or (iii) a colorimetric approach. Within the present disclosure, the lifetime approach uses a pulsed light-emitting diode (LED) or laser diode and monitors how the magnitude of the emission decays over time. From the decay curve, a lifetime can be calculated which corresponds to how much time the chromophore is in its excited state. As a practical matter, for a portable application, this is typically done for long-lived, phosphorescent chromophores. The intensity approach (which in one form is ratiometric) involves exciting the chromophore with an LED after which the emission takes place at another wavelength, although unlike the lifetime approach, it is the intensity (rather than the lifetime) changes of the emission in the presence of the analyte that are monitored. For a ratiometric intensity approach, there would also be an emission peak that is unchanged by the analyte and this emission intensity would be used as a reference. In some embodiments, the reference emission could be from a separate analyte-insensitive reference chromophore. In other embodiments, the reference emission could also be emitted from the analyte-sensing chromophore. By way of example, a dual luminescent chromophore could exhibit a dissolved oxygen-insensitive fluorescence emission and a dissolved oxygen-sensitive phosphorescence emission. In a ratiometric sensor, there could be, for instance, one luminescent chromophore for which the excitation wavelength maximum and/or emission wavelength maximum are shifted in the presence of the analyte. By way of example, in this instance, it may be possible to obtain a ratiometric measurement by exciting the chromophore at two different wavelengths while monitoring the emission intensity at the same wavelength in each case. The colorimetric (or color-changing) approach involves examining how the color changes in response to an analyte, specifically corresponding to changes in absorption as opposed to a luminescent (fluorescent or phosphorescent) emission. This approach can be quantified by exciting the sample with white light and monitoring the absorption of this light such as through a reflectance measurement. In other examples, quantification may take place through an observed physical color change. Regardless of the sensing approach being used herein, the interaction between the analyte and the light signal that takes place in the presence of the analyte-sensing chromophore causes the light signal to become altered in a way that can be detected and analyzed.

Thus, for certain types of sensing (such as those done to detect for the presence of lactate, glucose or dissolved oxygen, for example), the emission from the chromophore that interacts with the analyte of interest is detected in the form of an emission lifetime that is a measure of how long a molecule remains in an excited state prior to photon emission. Likewise, for other types of sensing (such as those done to detect for the presence of electrolytes or stress-related biomarkers), the intensity of the emission wavelengths from the chromophore are monitored. In the more specific ratiometric intensity case, the intensity of the emission wavelength from the analyte-sensing chromophore relative to an analyte insensitive reference is used to provide a ratiometric measure of the level of the analyte of interest. Moreover, and for other types of sensing, such as those done to detect for the presence of pH, a colorimetric approach includes an analysis which produces detectable color changes within the chromophore. In all of these approaches, changes in the characteristic emission from—or absorption of—the chromophore-activated array can be monitored by an external reader (such as a detector) and computer-based analysis to provide indicia that would correspond to the presence of the relevant analyte. These approaches, as well as the platforms used to perform analyte monitoring based on such approaches, will be discussed in more detail as follows.

A technical benefit of the method disclosed herein includes the ability to monitor dissolved oxygen and electrolytes, such as dissolved metal ion levels (including, for example, sodium, potassium and calcium) and others (such as chloride), in the body on demand that in turn allows the detection of early onset of hypoxia, hyperoxia and dehydration. In a similar way, the presence of certain biomarker-related compounds (such as cortisol, lactate, dehydroepiandrosterone sulfate (DHEAs) or neuropeptide Y (NPY)) that provide an indication of stress or fatigue may also be monitored, as can urea, creatinine or glucose. Monitoring these health-related biomarkers in real-time has a direct relationship with individuals that are in static or dynamic environments, such as patients being transported during a medical emergency as well as police, firefighters, first responders and military service personnel in addition to patients (that is, individuals that are under the care of a doctor, nurse, emergency treatment personnel or other medical professional and being diagnosed or treated for a particular medical condition), individuals that are participating in organized sports (such as those whose participation is sanctioned by a recognized professional, intercollegiate, high school or related governing body), research subjects (that is, animals or humans that are being monitored as part of a research study) and individuals who wish to monitor their own health. Likewise, in tactical situations, the knowledge gained on the individual may be used to aid in the understanding or assessment of unexplained physiological events (UPEs). Within the present disclosure, the term “analyte” is meant to generally describe a measurable component of interest in a chemical analysis, while various particular types—such as the aforementioned dissolved oxygen, electrolytes, biomarkers or the like—are used to describe the analyte with a greater degree of specificity. Such generality or specificity will be apparent from the context.

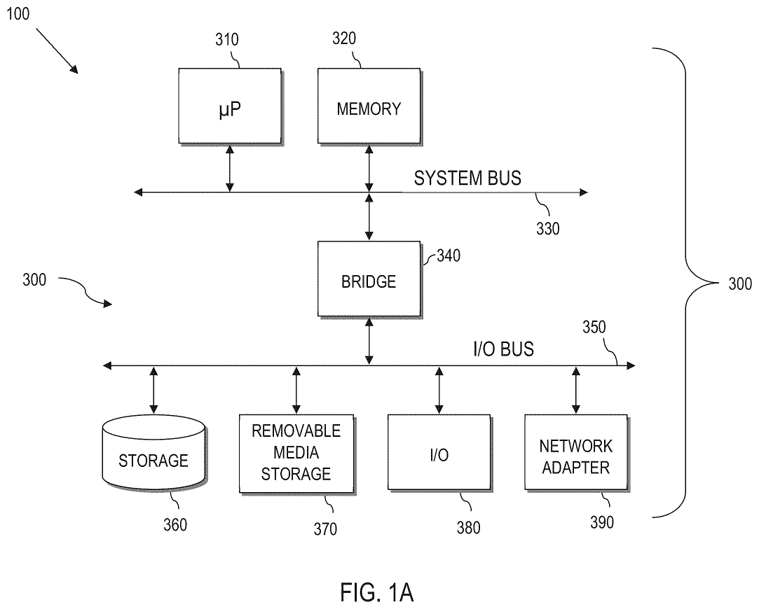

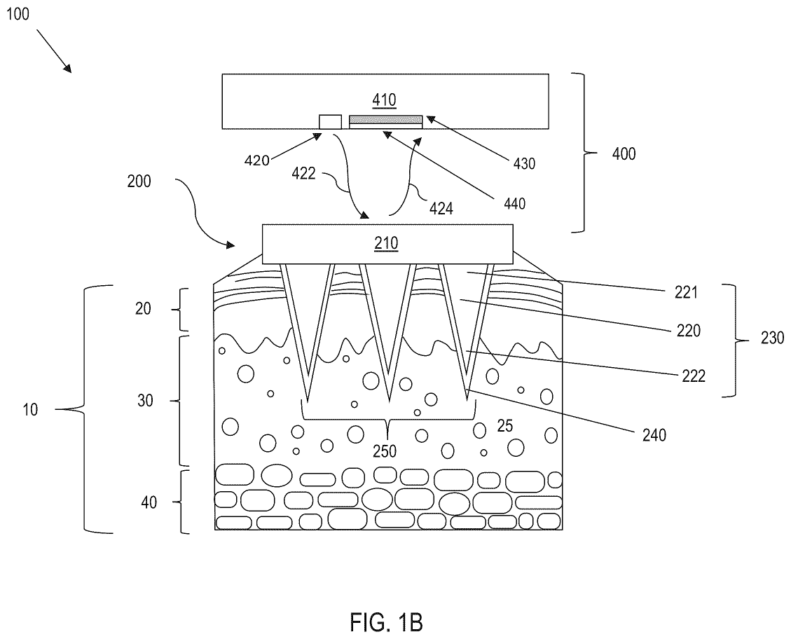

Referring first to FIGS. 1A and 1B, skin 10 of an individual is shown being monitored by an analyte-sensing system 100 with a notional skin-mounted optical microneedle assembly 200, where the various layers corresponding to the epidermis 20, dermis 30 and hypodermis (that is to say, subcutaneous) 40 of the skin 10 are shown in more detail. Signal cooperation between the analyte-sensing system 100 and the microneedle assembly 200 may be achieved through a control circuit that is in the form of a computer system 300. As discussed elsewhere, the various parts of the analyte-sensing system 100 may acquire data in the form of chromophore-transformed light signals that correspond to the presence of dissolved oxygen, electrolytes or other analytes of interest within the interstitial fluid and then apply algorithmic or library-based pattern recognition techniques on the acquired data as a way to analyze, store and report the results, such as to a clinician, caregiver, the individual being monitored or other person who is monitoring one or more of the physiological parameters of an individual.

In one form, the microneedle assembly 200—either in cooperation with or formed as a part of the computer system 300 or other part of the analyte-sensing system 100—is configured as a biosensor in that the analyte-sensing chromophore contained in its microneedles 220 function as a biosensing element that interacts with an analyte and experiences a change in at least one of its photophysical properties in response to the presence of the analyte, while a transducer element converts this response into a detectable signal. In such form, the analyte-sensing system 100 may likewise be arranged as a biosensor system. Furthermore, the microneedle assembly 200 configured as a biosensor may be configured as an internet of things (IoT) device where various connectivity, small form factor, pre-processing and low-power functionalities may be implemented. With such construction, the information acquired by the microneedle assembly 200 may be processed in such a way to allow the computer system 300 or other part of the analyte-sensing system 100 to be configured as an edge device for the at least partially decentralized acquisition, analysis and reporting. Relatedly, computational algorithms (which in one form may be stored as a program on a memory and operated upon by a processor both of which make up a part of the computer system 300) may be configured to cooperate with the microneedle assembly 200 to form a soft sensor. In a similar manner, the collection of data from numerous different individual microneedles 220 may be aggregated or fused in such a way that the computational algorithm uses the fused data to generate additional analytical insights.

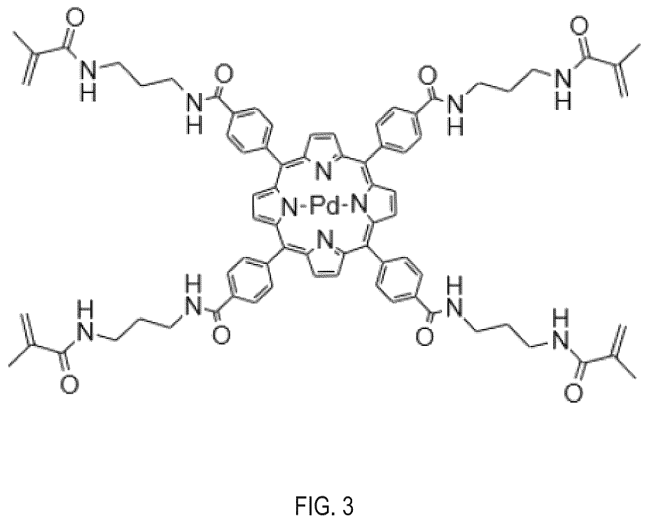

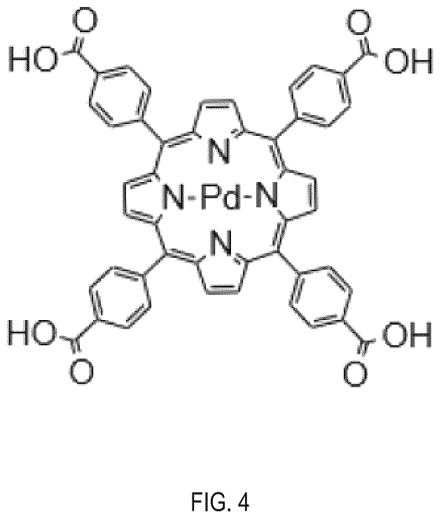

As shown, the microneedle assembly 200 is made up of a substrate 210, an array 250 of numerous individual optically transparent microneedles (of which only three are shown) 220 that terminate at their opposing ends in a base 221 and a tip 222 that together form a transdermal window 230, along with a biocompatible polymer (such as the aforementioned hydrogel) 240. In some embodiments, the microneedle array is formed on an adhesive patch such that upon the end of its useful life, it can be simply peeled off the skin and disposed. As will be discussed in more detail in conjunction with FIGS. 3 and 4, in one form the biocompatible polymer 240 is configured to contain an analyte-sensing chromophore. In some embodiments, the substrate 210 may be opaque or optically transparent. Thus, in the former case, any opaqueness that is present within the transdermal window 230 would ideally only be in the generally planar space that is in between the individual microneedles 220. In addition, the substrate 210 may be biocompatible (including being hypoallergenic or otherwise non-irritating), as well as have adhesiveness and flexibility. As shown, the individual microneedles 220 are formed and placed on a skin-facing surface of the substrate 210 such that a light signal being introduced transdermally comes either wholly or partially through the microneedles 220. For example, not all of the light need come through the microneedles 220 as there could be light going through the substrate 210 instead, as well as that reflecting off of one or more surfaces or the like. In addition, as shown, the individual microneedles 220 are cooperative with (such as by coating or the like) the biocompatible polymer 240 that may or may not be optically transparent. With particular regard to the biocompatible polymer 240, its features include—in addition to the hypoallergenic or otherwise non-irritating properties mentioned in conjunction with the substrate 210—the ability to allow the analyte being investigated to diffuse therein to interact with the chromophore to induce a change in at least one of its photophysical properties. In use, the substrate 210 is firmly pressed onto the outermost (stratum corneum) layer of the epidermis 20 that in turn causes the individual microneedles 220 (that in one example may have axial length that extends beyond the substrate 210 by between about 0.4 and 1.5 mm, and in a more particular between about 0.4 and 1.0 mm) to pass a distance sufficient through the outermost and subsequent layers of the epidermis 20 and extend deep enough into the dermis 30 such that they can sense analytes that are present in an interstitial fluid 25 that is present therein, but not so far that they extend into the hypodermis 40. Significantly, this relatively shallow penetration is conducive to the use of a chromophore that has photon excitation and emission within the visible range that in turn makes it easier to conduct lifetime, ratiometric or colorimetric analyses. In some embodiments, all of the individual microneedles 220 within the array 250 may be functionalized with the same biocompatible polymer 240.

Within the present disclosure, the substrate 210 may be made from the same material as the individual microneedles 220 or from a different material. As such, the microneedles 220 may be attached to a similar or dissimilar backing material, depending on the need. Relatedly, the microneedles 220 may be made as part of the same process and substantially at the same time (such as by co-forming or the like) as the substrate 210 or separately—i.e., fabricated and then subsequently joined. In yet another example, the substrate 210 may be dispensed with altogether such that the microneedles 220 may engage directly with the skin 10 without any intervening structure. It will be appreciated that all of these forms are within the scope of the present disclosure.

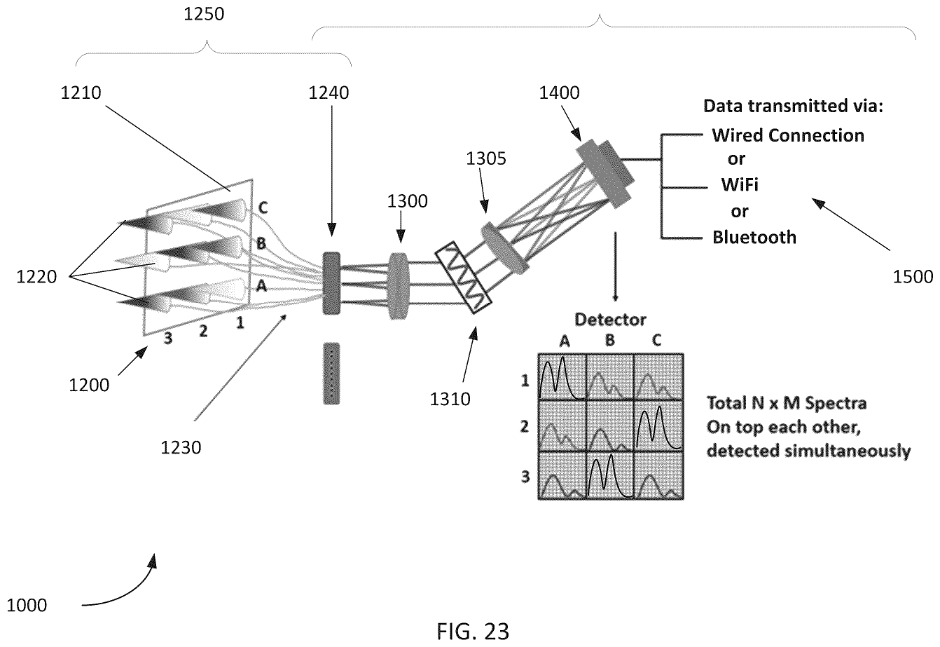

A reader (which may include a detector, or more particularly a photodetector, depending on the signal being sensed, as well as other components) 400 is shown attached to or otherwise situated on a surface of the substrate 210 that is opposite of the skin-facing surface such that in one form, the reader 400 and the microneedle assembly 200 may be configured as an integral whole, while in another as separate (but signally-cooperative) components where either form is within the scope of the present disclosure. In one example, the detector may be configured as a photomultiplier tube (PMT), photodiode or other signal-sensing device. Within the present context, the more general term “reader” is understood to include equipment that can introduce a light signal to the transdermal window 230 such as through the use of one or more LEDs or laser diodes, can detect an event within a given environment and (in certain configurations) can provide its own ability to provide intelligible content to the sensed data that corresponds to the event, while the more particular term “detector” merely senses the event for recordation or registration such that the light signal is introduced through one or more separate components, and the corresponding data may be conveyed to a computer or other processing equipment for subsequent analysis and intelligible content. In configurations (not shown) where the reader 400 is physically separated or otherwise remote from one or more of the substrate 210 and the microneedle assembly 200, the signal cooperation may be made to take place through known wired or wireless communication means. Various forms of the reader 400 may be used, including those configured as a charge-coupled device (CCD) and accompanying grating, a complementary metal-oxide semiconductor (CMOS) device, a photodiode (including those tuned to a particular wavelength range), an avalanche photodiode, a PMT or the like. Likewise, in one example, the reader 400 may be configured to have a miniaturized, conformal form factor in order to secure it to the substrate 210. In some example, the reader 400 may be partially adhered to the skin 10 of FIG. 1B, and partially to the substrate 210. The CCD and CMOS variants will be discussed in further detail in conjunction with FIGS. 23 through 27.

In some embodiments, the reader 400 may be configured to act as the light source as well as the aforementioned detector. In such configuration, the reader 400 may also include its own circuitry and components 410 that can be used to send light-based (that is to say, optical) signals through an optical exciter 420 to generate the light source and receive (or collect) returned light through an optical receiver 430, while in other embodiments (not shown), its functionality may be integrated into one or more separate components that make up the analyte-sensing system 100. More particularly, the optical receiver 430 includes functionality that allows it to either include or take the place of a conventional detector that, as previously noted, is in some embodiments a component of the reader 400. One or more optical filters 440 may optionally be included to be placed in signal cooperation with the optical receiver 430. In some examples, some or all of the circuitry associated with the operation of the reader 400 may be situated within the computer system 300. It will be appreciated that the analyte-sensing functionality of the reader 400 is the same, regardless of where the operational control or receipt of the optical signal resides, and that all such forms are within the scope of the present disclosure. Within the present context, it will be appreciated that the light signals being received into the optical receiver 430 may be through various known mechanisms after interaction of light signals with the chromophore, depending on the type of sensing (lifetime, intensity or colorimetric) being performed. Such mechanisms may include—but are not limited to—reflection from the analyte-sensing chromophore that is present in or on the microneedles 220 of a portion of the light signal being emitted by the optical exciter 420, as well as emission of a signal in response to the light signal from the optical exciter 420 that has been at least partially absorbed by the analyte-sensing chromophore that is present in or on the microneedles 220. By way of example, lifetime and intensity sensing generally involves detection of light that has been emitted by the chromophore, while colorimetric sensing generally involves detection of white light, a portion of which has been absorbed such that the remainder that is being reflected has a different spectral content than that of the original light signal being emitted by the optical exciter 420. Regardless of the mechanism, the light that is intended for receipt by the optical receiver 430 is generally referred to herein as returned light in order to distinguish it from the light that is originally emitted by the optical exciter 420, while more specific versions of such returned light will be apparent from the context.

The sending and receiving of optical signals between the reader 400 and the optically transparent microneedles 220 is part of a spectroscopic process to detect the presence of one or more analytes within the interstitial fluid. By way of example, the optical exciter 420 that acts as a light source of the optical signal for the reader 400 may include an LED or laser diode for which an emitted light signal 422 upon being transmitted through the microneedle assembly 200 can be absorbed by an analyte-sensing chromophore present in the biocompatible polymer 240. The analyte-sensing chromophore can interact with an analyte that is present in the interstitial fluid 25 and that has diffused into or otherwise interacted with the biocompatible polymer 240 that is present in or on one or more of the optically transparent microneedles 220 such that it induces a change in at least one photophysical property of the analyte-sensing chromophore. Thus, these changes may be identified through a comparison of the emitted light signal 422 to a returned light signal 424 that is sensed, measured or otherwise detected by the optical receiver 430. The analyte-sensing chromophore that is present within the biocompatible polymer 240 is optically responsive to a particular analyte within the interstitial fluid. Thus, when the emitted light signal 422 has interacted with the analyte-sensing chromophore within the interstitial fluid 25 in the presence of the targeted analyte for sensing in the hydrogel or other biocompatible polymer 240, the chromophore undergoes at least one photophysical change in the form of emission intensity, emission lifetime and/or color change that can be optically detected by the returned light signal 424. Circuitry contained within either the reader 400 or elsewhere (such as the computer system 300) may be used to correlate the presence of that particular analyte, such as through algorithmic determination or comparison to a known (baseline) reference signal such as those determined by a previous analyte spectrographic or spectroscopic analysis. Such circuitry allows the analyte-sensing system 100 to operate as a spectrometer in that the detection, subsequent regularization of the signals through an algorithmic, equation-based procedure and determined analyte value analysis and presentation may take place. Although not all are shown, the reader 400 may include other components in order to send, receive and process the light signals. Such components may include one or more filters (such as wavelength-selective filters or angle-of-incidence filters to avoid saturation problems), a diffraction grating, as well as one or more diode-based or capacitor-based imaging photosensors that are tailored to detect the light emitted or reflected by the chromophore that is contained within the biocompatible polymer 240. As such, the optical exciter 420 and optical receiver 430 may either or both be active or passive, depending on the degree of signal processing and analysis performed in the reader 400 versus that performed in the computer system 300 that is discussed in more detail next.

In some embodiments, the analyte-sensing system 100 is computer-based with functionality depicted as a schematic block diagram. In this way, the various activities related to the detection, analysis and reporting of the results may be performed automatically. The computer system 300 may function as a control circuit that may be used for implementing the various processes described herein, including where the computer system forms a part of a control circuit. The exemplary computer system 300 includes one or more (hardware) microprocessors (μP) 310 (or in the alternative, a system-on-chip microcontroller that itself forms an integrated circuit that also includes memory and one or more peripherals) for executing instructions that make up a computer program and corresponding (hardware) memory 320 (for example, random access memory and/or read only memory) that are connected to a system bus 330. Information can be passed between the system bus 330 (via a suitable bridge 340) and a local bus 350 that is used to communicate with various input/output devices. For instance, the local bus 350 is used to interface peripherals with the one or more microprocessors 310, such as storage 360 (for example, hard disk drives); removable media storage devices 370 (for example, flash drives, DVD-ROM drives, CD-ROM drives, floppy drives or the like); I/O devices 380 such as one or more of input devices (for example, mouse, keyboard, scanner or the like) and output devices (for example, monitor, printer or the like); and a network adapter 390. The present list of peripherals is mentioned by way of illustration and is not intended to be limiting. Other peripheral devices may be suitably integrated into the computer system 300. In a similar manner, all such devices and variants thereof are within the spirit and the scope of the present disclosure. In another form, the I/O devices 380 may include various display screens, such as those available on a computer, laptop, mobile telephone, smart watch, graphical user interface (GUI) or other related data-reporting device, including those placed upon or otherwise readily accessible to the individual being monitored. Such display screens cooperate with the computer system 300 such that a program stored in memory 320 and executed by the one or more microprocessors 310 can convey to a user an indication of the level of the analyte on the I/O device 380. It will be appreciated that while the sensing activities disclosed herein identify changes in the concentration of an analyte, the accuracy of a quantification of such changes could be impacted by various in vitro to in vivo circumstances, and that such circumstances, if need be, may be adjusted for, either algorithmically or otherwise.

The one or more microprocessors 310 control operation of the exemplary computer system 300. Moreover, one or more of the microprocessors 310 execute computer readable code (for example, stored in the memory 320, storage 360, removable media insertable into the removable media storage 370 or combinations thereof, collectively or individually referred to as computer-program products) that instructs the one or more microprocessors 310 to implement the computer-implemented processes herein.

The computer-implemented processes herein may be in the form of a machine-executable process executed on the computer system 300, or on any other structure described more fully herein.

Thus, the exemplary computer system 300 or components thereof can implement processes and/or computer-implemented processes stored on the memory 320 as well as one or more computer-readable storage devices 360, 370 as set out in greater detail herein. Other computer configurations may also implement the processes and/or computer-implemented processes stored on one or more computer-readable storage devices 360, 370 as set out in greater detail herein. Computer-program code for carrying out operations for aspects of the present disclosure may be written in any combination of one or more programming languages. The program code may execute entirely on the computer system 300 or partly on the computer system 300. In the latter scenario, the remote computer may be connected to the computer system 300 through any type of network connection, for example, using the network adapter 390 of the computer system 300.

In implementing computer aspects of the present disclosure, any combination of computer-readable medium may be utilized. The computer-readable medium may be a computer readable signal medium, a computer-readable storage medium, or a combination thereof. Moreover, a computer-readable storage medium may be implemented in practice as one or more distinct mediums.

A computer-readable signal medium is a transitory propagating signal per se. A computer-readable signal medium may include computer readable program code embodied therein, for example, as a propagated data signal in baseband or as part of a carrier wave. More specifically, a computer-readable signal medium does not encompass a computer-readable storage medium.

A computer-readable storage medium (such as memory 320 or the computer-readable storage devices 360, 370) is a tangible device/hardware that can retain and store a program (instructions) for use by or in connection with an instruction execution system, apparatus, or device, for example, a computer or other processing device set out more fully herein. Notably, a computer-readable storage medium does not encompass a computer-readable signal medium. Thus, a computer readable storage medium, as used herein, is not to be construed as being transitory signals per se, such as radio waves or other freely propagating electromagnetic waves through a transmission media.

Specific examples (a non-exhaustive list) of the computer-readable storage medium include the following: a hard disk, a random-access memory (RAM), a read-only memory (ROM), an erasable programmable read-only memory (EPROM), flash memory, a portable computer storage device, an optical storage device such as a compact disc read-only memory (CD-ROM) or digital video disk (DVD), or any suitable combination of the foregoing. In particular, a computer-readable storage medium includes computer-readable hardware such as a computer-readable storage device, for example, memory. Here, a computer-readable storage device and computer-readable hardware are physical, tangible implementations that are non-transitory.

By non-transitory, it is meant that, unlike a transitory propagating signal per se, which will naturally cease to exist, the contents of the computer-readable storage device or computer-readable hardware that define the claimed subject matter persists until acted upon by an external action. For instance, program code loaded into random access memory (RAM) is deemed non-transitory in that the content will persist until acted upon, for example, by removing power, by overwriting, deleting, modifying or the like.

Moreover, since hardware comprises physical element(s) or component(s) of a corresponding computer system (such as computer system 300), hardware does not encompass software per se.

It will be appreciated that the computer system 300 may be configured into numerous form factors all of which are within the scope of the present disclosure. For example, in one form, the computer system 300 may be configured as a stationary on-site or off-site stationary device, while in another form to be miniaturized such that it may take on a wearable form factor, such as being affixed to the individual, such as through wrist-mounted, arm-mounted, torso-mounted, leg or ankle-mounted configurations. It will be appreciated that other devices besides those being mounted to body parts, in addition to other than those listed, are still within the scope of the present disclosure. In still another form factor, the computer system 300 may be configured to be in the cloud. Communication within or by the computer system 300 may be through wired or wireless means where the latter may include known long-range or short-range approaches such as mobile telephony, WiFi, Bluetooth, nearfield communications (NFC), or the like.

The various components that make up the computer system 300 may be configured as a controller and include one or more of the various components, including the I/O 380 as a user interface, the one or more microprocessors 310 and memory 320 the last of which may contain program code, machine codes, native instruction sets, computer readable instructions or related data structures such that upon loading into the memory 320, the program code is particularly configured to execute one or more steps in a manner consistent with the methods disclosed herein. In addition, the controller may include a communications circuit (such as through the network adapter 390 or other communication means) that may be either wired or wireless such in when configured as the latter, it may include a suitable radio-frequency transceiver that can access an external network (not shown). Within the present disclosure, the program code will be understood to include the organized collection of instructions and computer data that make up particular application software and system software the latter of which may include operating system software and basic input/output that relates to the operation of the computer system 300, regardless of its form factor. This and other software (such as system software or application-specific software) provides programmed instructions that may be implemented on the one or more microprocessors 310 to allow it (or them) to interact with the computer system 300 or other computer-based equipment in order to perform one or more of the data acquisition, processing, communicating, analysis and related functions disclosed herein. For example, source code may be converted into executable form as machine code for use by the one or more microprocessors 310; such machine code is predefined (or configured) to perform a specific task in that it is taken from a machine language instruction set known as the native instruction set that may be part of a shared library or related non-volatile memory (including memory 320 and the removable media storage devices 370) that is specific to the implementation of the one or more microprocessors 310 and its (or their) particular Instruction Set Architecture (ISA). As such, software instructions such as those embodied in the corresponding portion of the machine code configure the one or more microprocessors 310 to provide the program structure and associated functionality as discussed herein.

In operation, once the array 250 of the microneedle assembly 200 is inserted into the layer of skin 10, the biocompatible polymer 240 becomes immersed within the interstitial fluid 25 that is present in the dermis 30. In this manner, analytes within the interstitial fluid 25 can diffuse into the biocompatible polymer 240 and interact with the analyte-sensing chromophores. For example, to be configured as a dissolved oxygen sensor, the microneedle assembly 200 and associated assembly and analyte-sensing system 100 is configured such that a phosphorescent chromophore sensitive to the presence of dissolved oxygen is contained in the biocompatible polymer 240. One or more colored LEDs or laser diodes that make up the source of light (whether on the reader 400 or from a separate device) are used to transmit a light signal into the microneedle assembly 200 such that upon interaction of the light signal with the analyte-sensing chromophore that is present in the biocompatible polymer 240, the absorbed part of the light signal by the chromophore allows the chromophore to enter into an excited (that is to say, higher energy) state. By way of example, following intersystem crossing, the chromophore exhibits a phosphorescent emission. However, dynamic quenching by dissolved oxygen results in the phosphorescence emission lifetime decreasing in accordance with the local dissolved oxygen concentration. The reader 400 monitors these changes in phosphorescence lifetime. These phosphorescence lifetime changes are then related (such as through computer-based algorithmic or comparison approaches) to corresponding changes in the tissue oxygenation of the dermis 25.

By way of example, in a situation where the wavelength of the light signal being sent by the reader 400 or other light source and received into at least one of the individual microneedles 220 of the array 250 that makes up a portion of the microneedle assembly 200 exhibits a peak wavelength between 505 to 525 nm (preferably 515 nm), the wavelength is absorbed by one of the porphyrin chromophores disclosed herein (such as in FIGS. 3 and 4), and the peak wavelength of the resulting emission is between 690 and 720 nm. It will be appreciated that other excitation and emission wavelengths may also apply, depending on the chromophores, light source and readers 400 being used, and that all such variants are within the scope of the present disclosure.

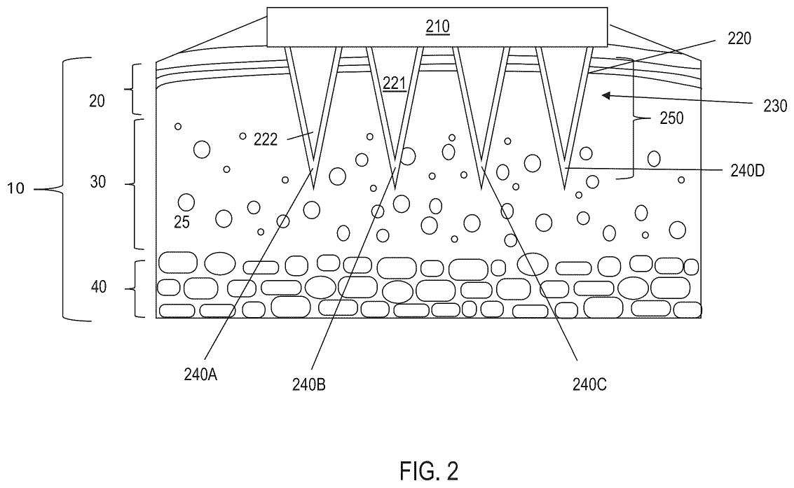

Referring next to FIG. 2, details of one particular type of microneedle array 250 made in accordance with the present disclosure are shown. As previously discussed, in one example, all of the individual microneedles 220 within the array 250 may be functionalized with the same biocompatible polymer 240, while in the embodiment presently shown, the biocompatible polymer 240 (as well as the analyte-sensing chromophore contained therein) may be tailored so that different individual microneedles 220 (along with one or more suitably-configured readers 400) may each detect a particular analyte within a broader range of analytes; the unique biocompatible polymers corresponding to such individually-tailored microneedles are shown as 240A, 240B, 240C and 240D, although it will be appreciated that this can extend to any number of individually-tailored microneedles 220. Relatedly, even within individual microneedles 220, striations or gradients along the height-wise dimension may be used to allow different sensing capabilities within a single microneedle. Thus, it will be appreciated that numerous coating patterns for the placement of the biocompatible polymer 240 are possible through the use of the various deposition techniques disclosed herein, regardless of whether the coating is (a) substantially the same among the numerous microneedles 220 that make up the array 250 or other large group, (b) individually tailored for each microneedle 220, or (c) tailored to have different functionality over the surface of each microneedle 220. In a related way, one or both of the microneedles 220 and the biocompatible polymer 240 may be coated with a selectively permeable material in order to improve the selectivity of the microneedle 220 for a certain analyte. In some instances, the membrane may selectively block the passage of certain other analytes for which the analyte-sensing chromophore may exhibit some response toward, thereby increasing the selectivity. In other cases, the membrane may selectively block the passage of larger molecules (e.g., proteins) to limit biofouling or other adverse effects. Selectively permeable coatings could either be commercially available or synthesized. Alternatively, the coatings may involve the incorporation of a commercially available or synthesized molecule into a polymer matrix. Example ionophores that could be commercially available and could help impart selectively via incorporation into a coating include sodium ionophore I, sodium ionophore II, sodium ionophore III, sodium ionophore IV, sodium ionophore VI, sodium ionophore X, calcium ionophore I, calcium ionophore II, calcium ionophore III, calcium ionophore IV, calcium ionophore V, potassium ionophore I, potassium ionophore III, chloride ionophore I, chloride ionophore II, chloride ionophore III and chloride ionophore IV.

As discussed elsewhere herein, analytes of interest may include one or more of dissolved oxygen, carbon dioxide, pH, markers related to stress or fatigue (such as lactate, DHEAs, cortisol or NPY), electrolytes (such as sodium, potassium, calcium, chloride), neurotransmitters (such as epinephrine or norepinephrine) as well as other biomarkers (such as urea, creatinine or glucose). As previously discussed, the use of the various sensing approaches (that is to say, lifetime, intensity (including ratiometric) or colorimetric) may be based at least in part on the analyte of interest. For example, monitoring for dissolved oxygen, lactate or glucose may employ a lifetime-based sensor, while monitoring for the various electrolytes, DHEAs, cortisol, NPY and the neurotransmitters may use an intensity (including ratiometric) sensor. In configurations where pH is being monitored, a colorimetric sensor may be used.

Referring next to FIGS. 3 and 4, two different porphyrin-based analyte-sensing chromophores are shown that may be used to detect dissolved oxygen from photoluminescence. In one form, these chromophores are configured as phosphorescent chromophores that are particularly configured to sense dissolved oxygen. The first example, Pd(II) meso-tetra(N-(3-methacrylamidopropyl)benzamido) porphyrin, is used in the polymer formulation to chemically bind the chromophore to the biocompatible polymer itself. For instance, the porphyrin can be crosslinked to a poly-2-hydroxyethyl methacrylate (pHEMA) hydrogel via in situ photopolymerization with a UV light source. Likewise, the porphyrin could be crosslinked into other acrylate-based polymers, such as poly(sulfobetaine methacrylate) (PSBMA) in the same manner. The example ensures leaching of the chromophore from the polymer is mitigated. Additionally, ensuring the chromophore is chemically bound to the polymer can help prevent chromophore aggregation as aggregation can affect its oxygen sensing capabilities. In the second example, Pd(II) meso-tetra(4-carboxyphenyl) porphyrin, is used when the oxygen sensing chromophore is simply blended into the biocompatible polymer and is therefore not chemically bound to the polymer itself.

Referring with particularity to FIG. 3, the Pd(II) meso-tetra(N-(3-methacrylamidopropyl)benzamido) porphyrin molecule is shown. In general, Group 10 elements and palladium (Pd) and platinum (Pt) elements in particular may be used to form a porphyrin organometallic complex such as those shown in FIGS. 3 and 4 that is known to exhibit certain photophysical properties. The presence of this heavy metal atom results in the molecule experiencing a large spin-orbit coupling that facilitates intersystem crossing, a process which—upon excitation—results in the transition from the singlet state to the triplet state. Thus, following light exposure, the radiative decay from this triplet state encompasses a phosphorescence emission. The lifetime of this phosphorescence emission is altered by the presence of dissolved oxygen which can dynamically quench the phosphorescence emission. Sensing of dissolved oxygen or other particular analytes by the coated optical microneedle array 250 that makes up a portion of the microneedle assembly 200 can be performed using an appropriate wavelength-sensitive reader. It will be appreciated that other analytes (with the exception of lactate and glucose) may use a different mechanism than the phosphorescence lifetime quenching mentioned here. For detection of lactate or glucose, the analyte in the presence of both a dissolved oxygen-sensitive phosphorescence chromophore (such as those shown in FIGS. 3 and 4) and an enzyme (e.g., lactate oxidase or glucose oxidase, respectively) could result in a decrease in the local dissolved oxygen concentration, causing an increase in the phosphorescence lifetime of the oxygen-sensitive chromophore. In this manner, the combination of the enzyme and the chromophore allow for an indirect determination of the analyte of interest, namely lactate or glucose.

Referring with particularity to FIG. 4, the Pd(II) meso-tetra(4-carboxyphenyl) porphyrin molecule is shown. As with the Pd(II) meso-tetra(N-(3-methacrylamidopropyl)benzamido) porphyrin molecule of FIG. 3, the Pd(II) meso-tetra(4-carboxyphenyl) porphyrin molecule may serve as an dissolved oxygen sensing chromophore contained within a biocompatible polymer. Unlike the chromophore of FIG. 3, the chromophore of FIG. 4 is not crosslinked into the polymer network.

When incorporated within the same biocompatible polymer 240 coating, the porphyrins shown in FIGS. 3 and 4 can both be used to sense dissolved oxygen and have similar photophysical properties and dissolved oxygen sensing capabilities. The resulting coatings may be expected to have similar deaerated lifetimes, Stern-Volmer quenching rate constants, bimolecular quenching rate constants, dynamic ranges, absorption maxima, emission maxima or the like. The main difference is that the porphyrin of FIG. 3 can be chemical bound to acrylate-based polymers, while that of FIG. 4 would just be blended into the coating.

Referring again to FIGS. 1A, 1B and 2, the individual microneedles 220 that make up the array 250 of the microneedle assembly 200 are made to be optically transparent through the choice of a suitable light-transmissive material. In some examples, the material is a polycarbonate or an adhesive such as Norland Optical Adhesive (for example, NOA63, NOA86, or NOA86H), although it will be appreciated that other materials that provide roughly comparable optical properties (such as glass, Loctite MED413 photopolymer resin, other Norland Optical Adhesives or the like) may also be used, and that all such materials are within the spirit and the scope of the present disclosure. These microneedles may be coated with a photopolymerizable mixture with either a 49:1:11:20:20 or 49:1:11:30:10 volume ratio of 2-hydroxyethyl methacrylate, triethylene glycol dimethacrylate, an at the most 10 millimolar solution of the analyte-sensing chromophore in dimethyl sulfoxide, deionized water or phosphate-buffered saline and ethylene glycol. In some embodiments, the biocompatible polymer 240 coating covers about 75% of the microneedle 220 by height from the top of the microneedles, where the thickness of the coating is between about 10 and 40 μm. In other embodiments, the individual microneedles 220 may have numerous different coatings formed along its elongated axial (that is height-wise) dimension. In that way, each individual coating may be configured to respond to a particular analyte such as dissolved oxygen, electrolytes or the other ones discussed herein. It will be appreciated that differences in the biocompatible polymer 240 coatings may arise out of different combinations, such as different chromophores but the same polymer, as well as other variations. Shape-wise, the individual microneedles 220 may resemble a pyramid, cone, obelisk or monolith with height along its axial dimension of between about 0.4 to 1.5 millimeters. In some embodiments, the microneedles 220 may be melt processed, such as injection molding, from known materials such as polycarbonate or high-density polyethylene. In other embodiments, the microneedles 220 may be made from photocurable hard resins such as the Norland Optical Adhesive product line, Loctite MED413 photopolymer resin or related photoresists. The microneedle tips 222 should be sufficiently sharp to allow skin penetration with tip resolution of 50 μm or less with microneedle base width between 50 to 700 μm. In some embodiments, the biocompatible polymer coating thickness ranges between about 4 to 45 μm and moreover may be highly localized to one or more of the individual microneedles 220. In addition, the coating of the biocompatible polymer 240 can be made to cover roughly 20% to 80% in height from the top of the individual microneedles 220.

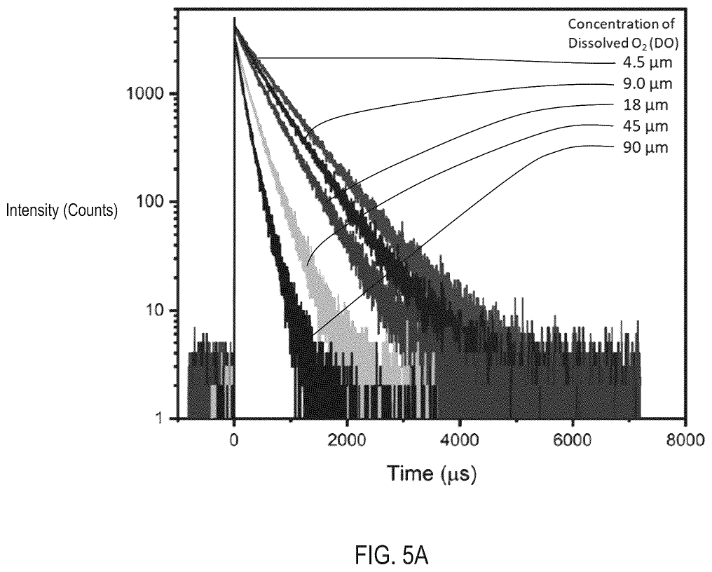

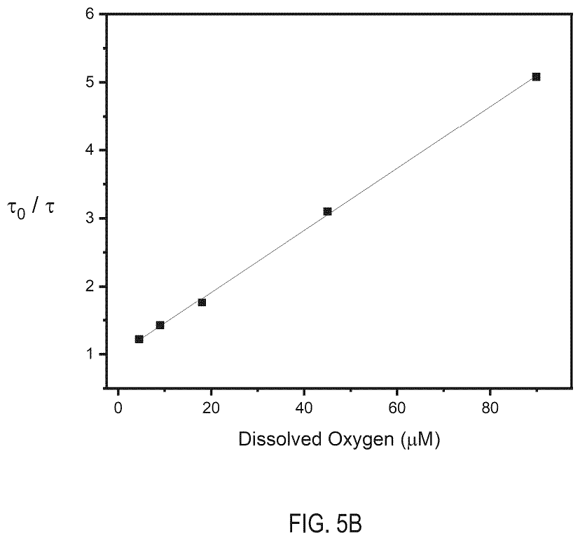

Referring next to FIGS. 5A and 5B, the emission lifetime decay of the phosphorescent chromophores of FIGS. 3 and 4 is influenced by the analyte (i.e., dissolved oxygen) concentration that is present within in the interstitial fluid 25 of FIGS. 1A, 1B and 2. As shown, the phosphorescence lifetime modulation is in response to dissolved oxygen was demonstrated by a version of the microneedle array 250 that has a poly-2-hydroxyethyl methacrylate hydrogel coating that contains the Pd(II) meso-tetra(N-(3-methacrylamidopropyl)benzamido) porphyrin of FIG. 3. This demonstration was done in vitro in 37 degrees Celsius (° C.) phosphate-buffered saline. The lifetime decays shown in FIG. 5A were fit with an exponential function to get the phosphorescence lifetime as a function of dissolved oxygen concentration. Those values were then used to make the Stern-Volmer plot of FIG. 5B that describes how the phosphorescence lifetime decreases as a function of the dissolved oxygen concentration.

Referring with particularity to FIG. 5B, a Stern-Volmer plot of the Pd(II) meso-tetra(N-(3-methacrylamidopropyl)benzamido) porphyrin-loaded hydrogel-coated microneedles sensors is shown. Thus, when the analyte-sensing system 100 (in general) and the computer system 300 (in particular) can perform pre-calibrated measurements of the analyte concentration in the interstitial fluid, using the Stern-Volmer relationship provides insight into the level of dissolved oxygen. The plot is made up of numerous lifetime data points, and in this particular instance shows that the chromophore in question helps produce an extremely long deaerated lifetime τ₀ of about 700 microseconds (μs). In some embodiments, an analysis of the amount of the analyte of interest within a given measurement may be compared to known (that is to say, calibrated) baselines, either algorithmically or through comparison to a lookup table or other form of spectral library. In addition to performing pre-calibrated measurements for in vitro measurements, other measurement procedures—such as those involving in vivo measurements—may instead not involve any pre-calibration.

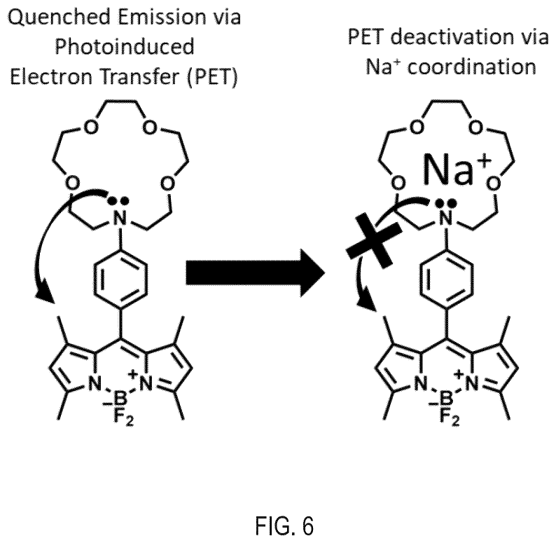

Referring next to FIG. 6, a mechanism is shown for sensing sodium ions via deactivation of photoinduced electron transfer (PET) in an aza-crown ether functionalized BODIPY-based chromophore. In general, BODIPY chromophores have highly modular properties with the potential for high signal output. In a manner similar to the chromophores of FIGS. 3 and 4, these analyte-sensing chromophores can be incorporated into a biocompatible polymer that in turn may be placed in or on one or more transparent microneedles 220. Upon excitation with a certain wavelength, the aza-crown ether functionalized BODIPY-based chromophore exhibits a fluorescence emission at another wavelength. In the absence of sodium ions, the lone pair of electrons on the aza-crown partially or fully quenches the fluorescence emission via PET. This aza-crown is designed to be a similar size to sodium ions. When Na⁺ binds the aza-crown, this reduces the effect of fluorescence quenching via PET. Thus, the fluorescence intensity of the BODIPY-based chromophore will increase based on the concentration of Na⁺ present in the interstitial fluid 25. Although not shown, it will be appreciated that an emission spectra of a N-phenyl-aza-15-crown-5-ether BODIPY chromophore loaded pHEMA hydrogel may be particularly used. In such case, the hydrogel may be soaked in an aqueous sodium chloride solution with various dissolved sodium chloride concentrations. Increasing the dissolved sodium ion concentration in the aqueous solution that the chromophore loaded hydrogel is soaking in leads to increases in the emission intensity.

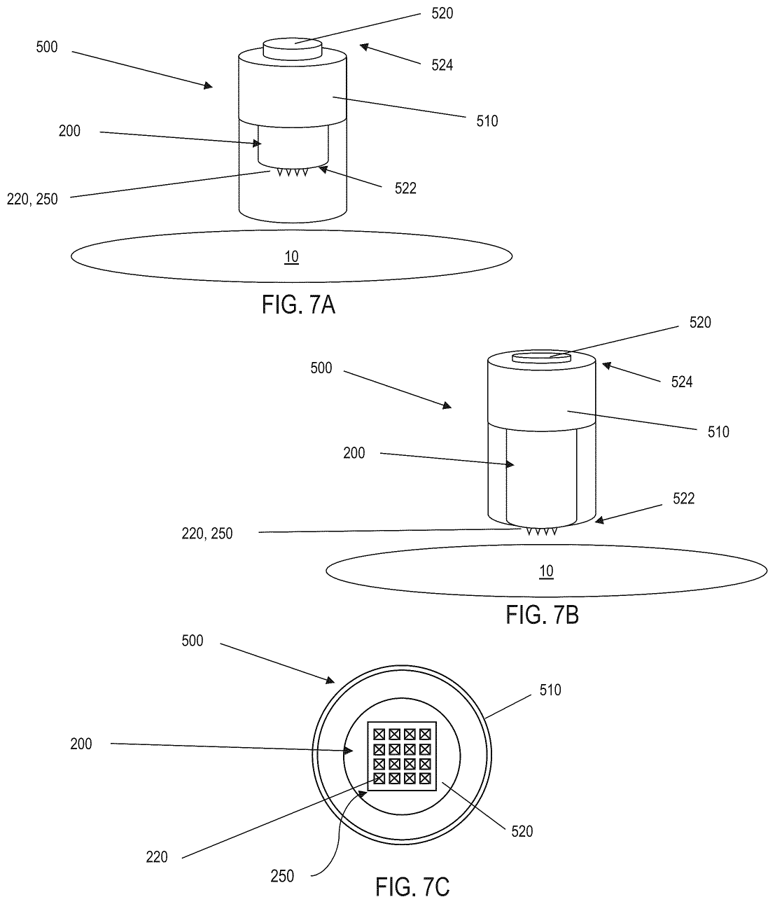

Referring next to FIGS. 7A through 7C in conjunction with FIGS. 1A and 1B, one form of transdermally analyzing an interstitial fluid is shown, where an application device 500 is used to first facilitate insertion of the microneedle assembly 200 (shown in its present embodiment as a four-by-four array 250 of individual microneedles 220) into the skin 10 of a patient. The application device 500 includes an outside casing 510 and a spring-loaded plunger 520 disposed therein with the microneedle assembly 200 affixed at a distal end 522 such that upon an actuating force (such as through thumb engagement or the like) being applied to a proximal end 524, the spring bias of FIG. 7A is overcome such that the microneedle assembly 200 moves in FIG. 7B along the downward axial dimension formed by the plunger 520 until the individual microneedles 220 pierce the epidermis 20 and dermis 30 the latter of which contains the interstitial fluid 25. Although not shown, it will be appreciated that other forms of application device 500 activation are possible, such as through other spring-based approaches, as well as proximal end 524 compression-based approaches, including those where the microneedle assembly 200 is already placed on the skin 10 after which the applicator device 500 is brought into contact with the microneedle assembly 200 in order to effect the desired piercing. FIG. 7C shows with particularity a bottom view of the application device 500 and the four-by-four array 250 of individual microneedles 220 that make up the lowermost portion of the microneedle assembly 200.

In some embodiments (not shown), a platform for colorimetric-based, intensity-based or lifetime-based testing of microneedles such as depicted in the microneedle assembly 200 may be used. For lifetime-based sensing, one testing platform may be used that allows for the acquisition of lifetime decays as a function of the analyte concentration in the surrounding solution. For intensity-based and colorimetric-based sensing, the testing platform is configured to either periodically or continuously acquire various reflectance or emission spectra. In both instances, the temperature of the solution and the concentration of analytes in the solution can be controlled. Significantly, the testing platform may utilize a photoluminescence spectrometer and related accessories to allow for evaluation of the sensor performance in a physiologically relevant environment while avoiding the need for an expensive, custom-built reader for this initial evaluation.

In the case of in vivo measurements, a portable (preferably wearable) reader platform may be used instead. The general goal is to have a reader with a detector and light source that could be adhered to the top of the microneedle array 250 that is inserted into the subject's skin. Based on the analyte that is being sensed, the mechanism of sensing being exploited, and the chromophore that is used to enable the optical sensing, different reader features are required. For instance, when sensing dissolved oxygen, one or more pulsed LEDs or laser diodes may be used as a light source, and a photodetector may be used to monitor the lifetime of the phosphorescence emission. In such case, a filter may be placed signally upstream of the photodetector so that the photodetector primarily captures light emitted from the chromophore. For measurements that are colorimetric- or intensity-based, the detector ideally captures a spectral window with a CCD and an appropriate grating. For colorimetric measurements, the light source is generally one or more white LEDs or miniaturized halogen sources, and the measured output is the reflectance as a means to quantify the color change. For intensity measurements, the light source is one or more colored LEDs or laser diodes, and the measured output is the emission spectra that ideally (in the case of a ratiometric intensity sensor) captures the analyte-sensitive emission of both the analyte-sensing chromophore as well as some analyte-insensitive reference emission such as that of a reference chromophore that is insensitive to the presence of the analyte. For both colorimetric and ratiometric intensity measurements, an alternative to a CCD and grating, could be two or more photodetectors covered by different bandpass filters to capture light intensity in two or more different narrow wavelength ranges. In the case of a non-ratiometric intensity measurement, only a single photodetector may be necessary.

These lifetime, intensity and colorimetric measurements will determine some of the functional elements that make up the reader 400. As previously noted relative to FIGS. 1A and 1B, the reader 400 may include its own circuitry 410 that can be used as the light source (to send) as well as the detector (to receive) such that the excitation light is sent through the optical exciter 420 and analyte-specific data is received through the optical receiver 430. For an LED- or laser diode-based configuration as previously discussed that is being used for luminescent (which includes one or both of fluorescent or phosphorescent) analyte sensing chromophores, the LED signals, for example, being emitted may be colored such that they correspond to an excitation wavelength for the particular chromophore. In this case, the optical receiver 430 may also include a filter 440 that is disposed along the wavepath of the signal to control the wavelength of light detected. In some embodiments, the filter is configured as a bandpass filter that is centered near the emission maxima of the luminescent analyte-sensing chromophore. In some embodiments (which are relevant for ratiometric intensity sensors), the reader 400 could be configured with multiple-colored LEDs or laser diode, in addition to one or more detectors, to detect emission of two different chromophores. In other embodiments, a single LED or laser diode type could be used to elicit both the analyte-sensitive emission and analyte-insensitive reference emission. In other embodiments, the reader 400 is configured to allow for detection of the spectral output of the one or more chromophores so that the emission spectra of intensity versus wavelength may be ascertained.

As previously discussed in conjunction with FIGS. 1A and 1B, during operation, the emitted light signal 422 from the optical exciter 420 is introduced into the transdermal window 230 that is formed by the individual microneedles 220 and the biocompatible polymer 240 that contains the analyte-sensing chromophore. In this situation, when at least a portion of the emitted light signal 422 is received into the transdermal window 230, the analyte causes a change to at least one photophysical property of the chromophore that in turn may be sensed with the emitted or reflected light signal 424. Examples of changes in photophysical properties that may take place and can be detected include changes in phosphorescence lifetime, changes in phosphorescence intensity, changes in fluorescence intensity, changes in peak emission wavelength, and changes in absorption.

With particular regard to changes in phosphorescence lifetime, the LED(s) or laser diode(s) of the optical exciter 420 are pulsed and the detector of the optical receiver 430 is used to obtain the decay curve seen after the excitation coming from the LED(s) or laser diode(s) turns off. This decay is in the form of emission intensity versus time. This data is then fit with a decay function such as:

I=I₀+e^{(-t/T)}

to obtain the lifetime T. In the foregoing equation, I represents the measured intensity at time t, and I₀ is the background intensity. With particular regard to changes in fluorescence or phosphorescence intensity, the LED(s) or laser diode(s) are on during measurement and the output is the intensity of the emission. Likewise, with particular regard to colorimetric changes, the light source(s) are white and are constantly on during measurement. In this manner, the amount of light that is not being absorbed and therefore making it back to the detector (such as through reflection or similar mechanism) is quantified.

In a simplest iterative form for use in colorimetric sensing, the reader 400 is configured to have two detectors (such as the aforementioned PMT or photodiode) that have bandpass filters to allow for detection of two different wavelengths. In more comprehensive forms, the detector acquires a spectrum of the reflected light signal 424 that corresponds to the intensity of light reflected back to the detector versus wavelength. Likewise, for ratiometric (intensity) sensing, there is a reference emission at another wavelength, and this emission is insensitive to the targeted analyte. The reference emission could either be from a separate analyte-insensitive reference chromophore or could be an analyte-insensitive emission from a dual luminescent chromophore. In the case of ratiometric sensing, the detector configuration of the reader 400 is similar to that used for the colorimetric case. Namely, there is either two detectors each with bandpass filters that allow for detection at two different wavelengths or there is a detector (i.e., CCD and grating) that allows for detection of part or whole of the spectra for both the reference emission and analyte-sensitive emission.

With particular regard to the reference emission, in a relatively simple case of intensity-based sensing, only the emission intensity of the analyte-sensing chromophore is monitored and there is no such reference emission. Such a detection scheme may be possible in cases when the specimen is not moving (for example, an animal under anesthesia). In such a case, an initial verification of the analyte level through another means (for example, a blood test) may be desired to verify the concentration at the start of the test. However, for real-world applications where the specimen is live and active, a specific kind of intensity-based sensor—namely, a ratiometric sensor—may be desired. With a ratiometric sensor, there may be a reference analyte-insensitive chromophore in addition to the analyte-sensing chromophore or there may be a dual luminescent chromophore that exhibits both an analyte-sensitive emission and an analyte-insensitive emission. In the former case, it will be appreciated that both chromophores would be contained within the biocompatible polymer. The analyte-sensitive and analyte-insensitive emissions are at two different wavelengths. The intensity of the analyte-sensitive emission relative to the intensity of the reference emission is then monitored. Thus, this manner of sensing can be used to account for some of the variability of the analyte-sensitive intensity that may arise in real-world applications.

In one configuration, the reader 400 transmits information wirelessly (such as through Bluetooth, WiFi, mobile telephony or other suitable communication protocols) to the computer system 300 or other hardware using suitable protocols and hardware and software interfaces (such as a mobile app, web app or the like). In another configuration, wired connectivity between the reader 400 and the computer system 300 or other backhaul may be provided. Factors based on how certain data initiation, gathering, processing and displaying take place may influence the physical situs of various components and functionality so that in one form, much of the activity takes place in a wearable platform. In this way, a device functions in an edge-like manner where much of the functionality is in, on or around the microneedle assembly 200 and wearable reader 400, while in another form, a significant amount such activity takes place in the computer system 300 (which in one form may take place in the cloud or as part of a server-based architecture neither of which are shown).

As previously noted, numerous ways to make an optical microneedle assembly 200 for use in interstitial fluid are disclosed, including aerosol jet printing, airbrush coating and inkjet printing. A summary of some of the features of the various methods of coating one or more of the microneedles 220 that make up the microneedle assembly 200 is described next. In some embodiments, the coating includes combining the analyte-sensing chromophore with a hydrogel.

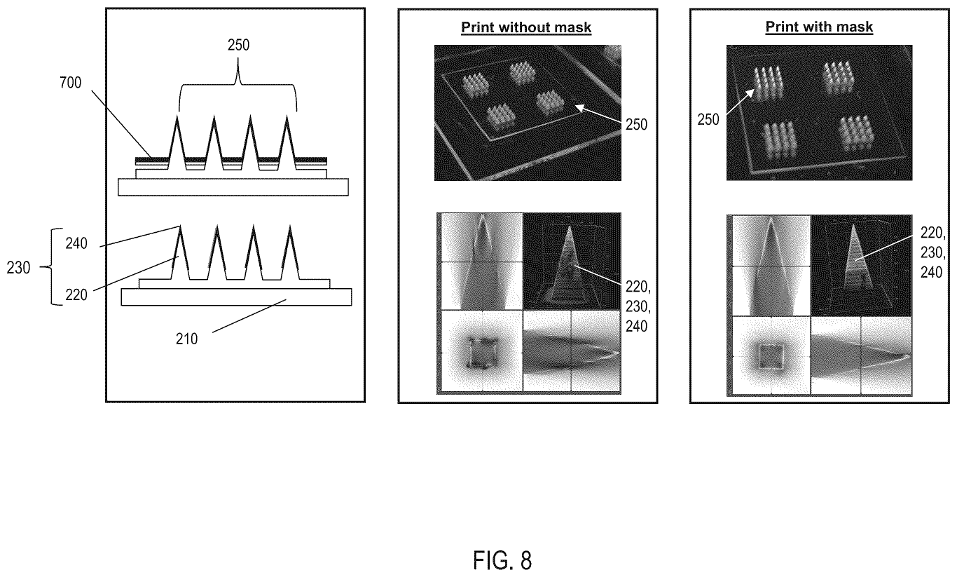

With particular regard to aerosol jet printing, patterns may be formed on the microneedle assembly 200 such that certain subsets of individual microneedles 220 are coated with a different formulation. While the use of a mask similar to mask 700 (which is shown in top portion on the leftmost frame of the figure FIG. 8) is not strictly necessary to prevent coating of the substrate or restrict coating to a certain region of the microneedle 220, in certain situations, the mask 700 may be used to more readily achieve these objectives. As previously mentioned in conjunction with FIG. 2, it may also be possible to allow for patterning of different formulations onto the same individual microneedle 220. One way this could be achieved could is to use a series of masks 700. One benefit of the aerosol jet printing approach is that it is fairly reproducible.

With particular regard to airbrush coating, because it is generally not amenable to microneedle 220 patterning, the use of a mask similar to mask 700 is necessary to localize coating deposition. Significantly, for small sample sizes, the setup and coating deposition may be made to take place rapidly.

With particular regard to inkjet printing, it inherently permits ease of patterning such that certain subsets of individual microneedles 220 are coated with different formulations and—like the aerosol jet printing approach—does not necessarily require the use of the mask 700 in order to achieve localization of the deposited coating made up of the biocompatible polymer 240. In this regard, the use of deposition control ensures that placement of the layer of the ink solution is restricted to a limited, targeted area in a manner similar to that which can be achieved with masks. Significantly, by optimizing this approach, masks (such as mask 700) may not be needed at all. Nevertheless, and like the aerosol jet printing approach, inkjet printing may also use a series of masks to allow for patterning of different formulations onto the same individual microneedle. Moreover, the reproducibility for this approach is high, while the setup and overall speed of the process is good. This technique is also industrially relevant and easily scalable. It will be appreciated that although the term “layer”, “layers” or the like as used in the present disclosure refers to a certain thickness of the coating of biocompatible polymer 240 that is applied to some or all of the surface of the one or more microneedles 220, when used within the context of the inkjet printing approach, the resulting formed layer is in fact an agglomeration of numerous individual droplets some of which during deposition may exhibit some degree of overlap and others of which may not obtain complete surface coverage. As such, and to the extent that the resulting coating may possess some measure of such overlap or incomplete coverage that leads to surface unevenness or the like, it still is deemed to be in the form of a layer.

Additional details associated with each of these coating deposition methods of the biocompatible polymer 240 will be discussed in more detail as follows.

Referring next to FIG. 8, the previously-mentioned inkjet approach for forming a hydrogel coating is shown in more detail. In some embodiments, a mask 700 is used as part of a masking-based printing procedure, while in another the printing may take place without the mask 700. Regarding this latter approach, various optimization strategies may be used as an overall inkjet coating simplification process for specified coverage of the needle (such as 70% coating coverage from the tip 222 of the microneedle 220). Otherwise, the mask 700 may be utilized to guarantee the preferred and specified coating coverage. It will be appreciated that particular print patterns will depend at least in part on the dimensions and design of the microneedles 220 to obtain the preferred coating area coverage and thickness. For instance, the areas that are being coated, the drop spacing (that is, the drops per inch) and the number of passes can be modified. The ink formulation, as well one or more of the print rate, print head temperature, nozzle temperature and build plate (that is to say, substrate) temperature can also be modified. Inkjet printing with adjustments to these parameters can be accomplished with commercially available printers an example of which is the FUJIFULM Dimatix Materials Printer. The volumetric output per layer is based on droplet spacing (for example, dots per inch) that may be adjusted as a parameter on the inkjet printer. By way of non-limiting example, the range of spacing of the droplets may be between about 5 and 50 μm (about 5080 to 508 drops per inch), and more particularly between about 5 and 20 μm (5080 to 1270 drops per inch) and even more particularly to about 10 to 15 μm (2540 to 1693 drops per inch).

Within the present disclosure, if the hydrogel or related biocompatible polymer that is coated onto, formed within or otherwise applied to the one or more microneedles 220 further requires some form of curing, it will be understood that such curing (or post-curing) may take place while the one or more microneedles 220 are in a upright position with the base in a relative downward position and the apex in a relatively upward position or in an inverted position (that is, with the base in a relative upward position and the apex in a relatively downward position). In this way, after an ink that contains the analyte-sensing chromophore is deposited (such as through airbrush coating, aerosol jet printing or inkjet printing) onto the one or more microneedles 220, the microneedles 220 (which in one form define a tapered shape from their base to their apex) may be placed with the base in a relative upward position and the apex in a relative downward position such that the effect of gravity on any as-yet uncured ink will be to flow away from the base.

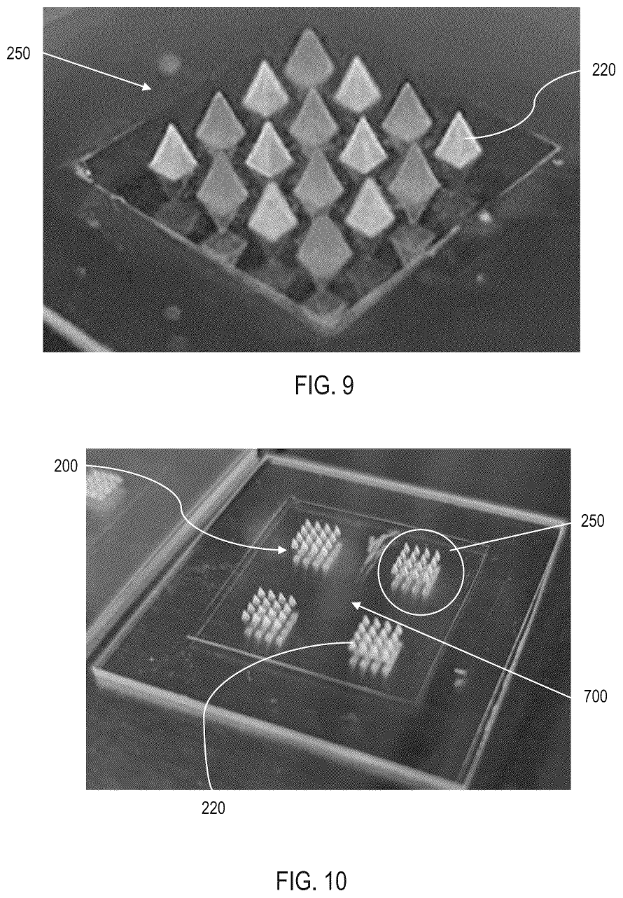

Referring next to FIG. 9, as previously noted, in one form, the individual microneedles 220 of the microneedle assembly 200 may be configured to have specific (or tailored) analyte-sensing capability—such as that mentioned in conjunction with FIG. 2—through the inkjet approach in order to provide the microneedle array 250 with numerous different patterns. Specifically, although a checkered pattern is shown, it will be appreciated that other forms, such as patterning by rows, columns, array quadrants or any other such pattern is deemed possible and within the scope of the present disclosure. In some embodiments, the biocompatible polymer 240 may be in the form of a hydrogel coating where one or more of the various microneedles 220 may have an analyte-specific sensing functionality.

Referring next to FIG. 10, a notional example of numerous arrays 250 of individual microneedles 220 are shown where the biocompatible polymer 240 of FIG. 1B, in the form of a hydrogel, was added by airbrush coating in conjunction with a laser cut mask that in one example is generally similar in construction to the mask 700 that is depicted in FIG. 8. The construction of the microneedles is such that they are transparent to radiation within at least the 400 through 700 nm part of the electromagnetic spectrum. In some embodiments, the biocompatible polymer coating ink formulation precursor containing an analyte-sensing chromophore is cured upon ultraviolet (UV) light or thermal exposure after airbrush deposition. In some embodiments, the microneedle array 250 may be made by airbrush coating with the laser cut mask 700. The size of the openings may be configured to the degree of coverage desired. For example, each of the openings may be made as a 390 micrometer (μm)×390 μm in one example, and as a 290 μm×290 μm in another.

The composition of the hydrogel network may include a master hydrogel formulation that has either a photocurable formulation or thermally curable formulation; examples of both are described in more detail as follows. In any formulation, there will be the presence of about 0.5 weight percent of initiator including, but not limited to, Irgacure 184, Irgacure 651, and Irgacure 819 photoinitiators and benzoyl peroxide and azobisisobutyronitrile thermal initiators. For a first photocurable formulation A, a photopolymerizable mixture is made with either a 49:1:10:19.5:20 or 49:1:10:9.5:30 volume ratio of 2-hydroxyethyl methacrylate, triethylene glycol dimethacrylate, an at the most 10 millimolar solution of the analyte-sensing chromophore in dimethyl sulfoxide, deionized water or phosphate-buffered saline and ethylene glycol. In this case, the resultant hydrogel is pHEMA. Likewise, for a second photocurable formulation B, the mixture is an 82:1:11:20:20 volume ratio of [2-(methacryloyloxy)ethyl]dimethyl-(3-sulfopropyl) ammonium hydroxide, 3-[Bis[2-(methacryloyloxy)ethyl](methyl)ammonio]propane-1-sulfonate, an at the most 10 millimolar solution of the analyte-sensing in dimethyl sulfoxide, deionized water or brine and ethylene glycol. In this case, the resultant hydrogel is PSBMA. In addition, for a thermally curable formulation, the mixture is a 9.5:26:1:1 weight ratio of an at the most 10 millimolar solution of the analyte sensing chromophore in ethanol, phosphate-buffered saline, polyethyleneimine (branched, molecular number average of 10,000 kDa) and poly(ethylene glycol) diglycidal ether. In this case, the resultant hydrogel is polyethyleneimine (PEI).

It will be appreciated that other precursors for the hydrogel may also be used or that the resulting mixture may vary from the aforementioned formulations. Such precursors may include 2-hydroxyethyl methacrylate, triethylene glycol dimethacrylate, [2-(methacryloyloxy)ethyl]dimethyl-(3-sulfopropyl) ammonium hydroxide, 3-[Bis[2-(methacryloyloxy)ethyl](methyl)ammonio]propane-1-sulfonate, polyethyleneimine and poly(ethylene gycol) diglycidal ether. The use of each of these as ingredients for the hydrogel, either individually or in combination, are deemed to be within the scope of the present disclosure.

The microneedle array 250 is generally made from either a negative polydimethylsiloxane (PDMS) mold using a commercially available, curable hard resin or melt processed into a master negative mold with a molten commodity polymer such as polycarbonate. Some or all of the formed individual microneedles 220 are then coated with a biocompatible polymer formulation that in one form is made up of either photocurable hydrogel formulation A or B or thermally cured hydrogel formulation as previously described. Deposition of these hydrogel coatings can be done through aerosol jet printing, inkjet printing or airbrushing. In one instance, a laser cut mask 700 similar to the one depicted in FIG. 8 may be placed on the individual microneedles 220 to ensure that deposition is restricted to the targeted areas.

In some embodiments of the aerosol jet printing approach of photocurable formulations, a 3:1 volume mixture of either photocurable formulation A or B to ethanol is used as the aerosol jet ink with aerosol jet printing parameters of 100 standard cubic centimeters per minute (sccm) sheath flow, 50 sccm atomizer ink flow, 2 second dwell time, 20 repeat coatings per microneedle 220 and build plate temperature at 60° C. while exposed to ultraviolet (UV) light for in situ photocuring. The microneedles 220 are then subjected to a post-cure in a UV chamber for 6 minutes while inverted to prevent any dripping of the ink towards their base 221. In some embodiments, aerosol jet printing of the thermally curable formulation may be accomplished with a 2:1 volume mixture of the thermally curable formulation master to ethanol with aerosol jet printing parameters of 50 standard cubic centimeters (sccm) sheath gas flow, 50 sccm atomizer ink solution flow, 1.5 second dwell time, and 5 repeat coatings per needle and build plate temperature at 50° C. In both forms, the sheath gas is used to surround the ink solution flow to help shape it, while the atomizer flow rate controls the extent of ink deposition.

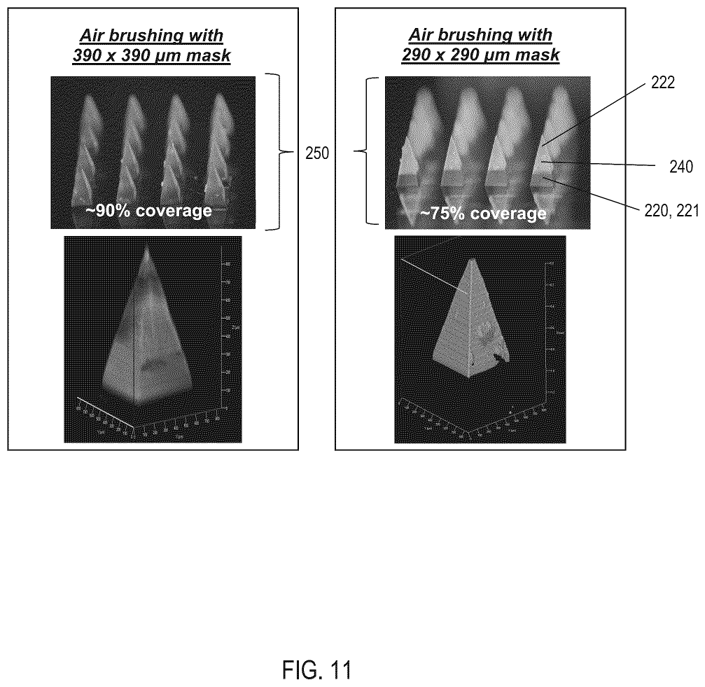

Referring next to FIG. 11, magnified optical and fluorescence confocal images of two different sets of the hydrogel-coated microneedles 220 of FIG. 1B are shown. As previously noted in conjunction with FIG. 2, numerous different patterning approaches may be used for the microneedles 220, either on an individual basis (where each microneedle 220 may be coated differently from its neighbors, or even differently over various parts of its surface, such as along its length) or as part of the array 250 (where one array 250 may be coated differently from its one or more neighbor arrays 250). Thus in the example depicted in FIG. 11, the coating that includes the biocompatible polymer 240 can be patterned in a way that limits coverage to the top portion (that is to say, at or near the tip 222) of the individual microneedles 220 through the use of a version of the laser cut mask 700 of FIG. 8 at a specified dimension of the mask 700 size. In the example using a version of the mask 700 with 390 μm by 390 μm hole features, the resulting biocompatible coating 240 is limited to approximately 90% coverage of each of the microneedles 220. Upon use of a version of the mask 700 with the smaller 290 μm by 290 μm hole features, this coating coverage is adjusted to an even more limited coating of approximately 75% coverage of each microneedle 220. In some embodiments, the biocompatible polymer coating 240 containing the analyte-sensing chromophore is localized to the tip 222 of each microneedle 220 to ensure appropriate optical signal detection is in the presence of the interstitial fluid 25 of FIGS. 1B and 2.

While it will be appreciated that avoiding the deposition of the biocompatible coating 240 onto one or more of the substrate 210 and the base 221 of the microneedle 220 is generally preferable, it is particularly beneficial for the detection of dissolved oxygen when interference from environmental oxygen would affect the accuracy of the measurement. Thus, when configured to perform dissolved oxygen detection in the interstitial fluid, by using phosphorescent chromophores with a long-lived emission, such as Pt (II) and Pd(II) porphyrins, the resulting biocompatible polymer 240 coating could also exhibit changes in phosphorescence lifetime as a result of being in the presence of any environmental oxygen. It will be appreciated that besides this example that is associated with the lifetime sensing that corresponds to dissolved oxygen detection in the interstitial fluid, other forms of sensing (such as the intensity sensing and colorimetric sensing of the same or other analytes) may also benefit from the avoidance of indiscriminate placement of the coating on the substrate 210 that, like oxygen, can lead to interference of analytes not originating from the interstitial fluid. By way of example, for the intensity-based detection of electrolytes, it may be beneficial to avoid the presence of the analyte-sensing chromophore at one or more of the base 221 of the microneedle 220 and the substrate 210 in case this could interact with sweat on the surface of the skin 10 as the sweat could also contain a different quantity of the electrolyte. By way of example, for colorimetric-based detection of pH, it may also be beneficial to avoid the presence of the chromophore near the base 221 of this microneedle 220 and on the substrate 210 to avoid interference from sweat.

For the airbrush approach, facile airbrush deposition and localized biocompatible polymer (for example, hydrogel) coating of the tip 222 of the various microneedles 220 is achieved through use of a laser-cut polymer film. By cutting very small and well-defined apertures in the film to make the masks 700, a certain height of the microneedle 220 can be exposed to the biocompatible polymer 240 deposition. Prior to or following the removal of the mask 700 (shown in FIG. 8), the deposited biocompatible polymer formulation can be cured (such as through a 10-minute UV exposure for the photocurable formulations, or a two-hour thermal curing at 60° C. for the thermally curable formulation). Thus, the size and dimensions of the laser-cut apertures in the thermoplastic film allows selective and limited-area coating of the microneedles 220. In this way, forming the biocompatible polymer 240 on a plurality of microneedles occurs only over a portion of each of the one or more microneedles 220 being treated. Curing (such as through thermal curing, photocuring or the like) of the hydrogels or other biocompatible polymer 240 coatings may be used.

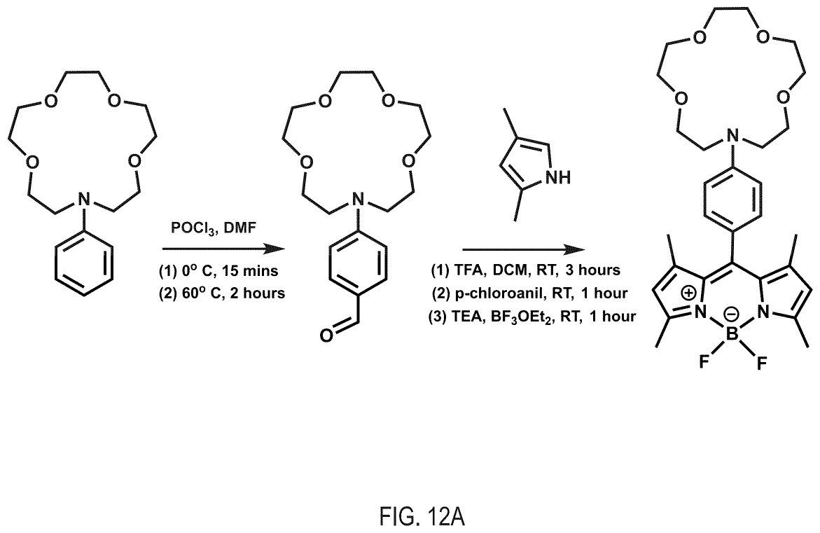

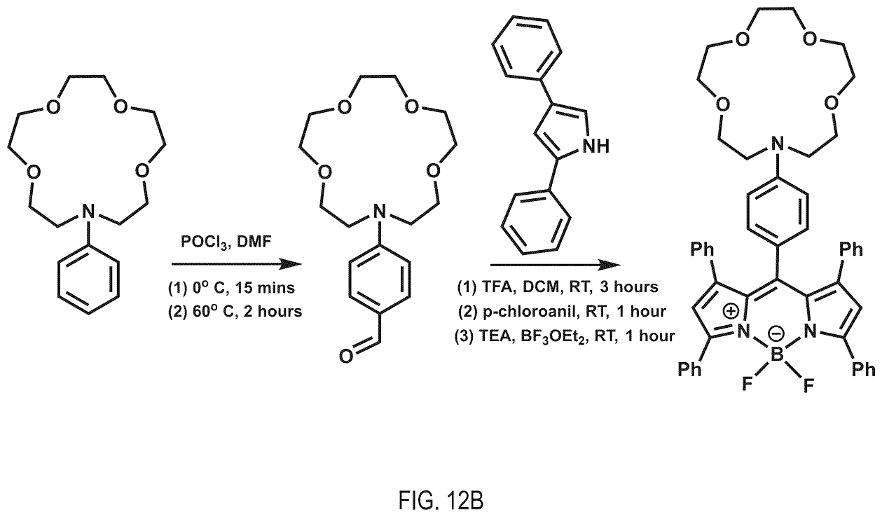

Referring next to FIGS. 12A and 12B, a synthetic approach for a metal electrolyte (specifically sodium, Na⁺) sensor is shown. As shown, an overall two-step synthesis approach is used to make the BODIPY-based chromophore designed for metal electrolyte sensing that is similar to the synthesis of known BODIPY based chromophores. The first step involves mixing phosphorus oxychloride (POCl₃) in dimethylformamide (DMF) at 0° C. for 15 minutes followed by the addition of a dilute solution of N-phenylaza-crown ether (specifically N-phenylaza-15-crown-5 ether for sodium, Na⁺) in DMF for two hours at 60° C. The phenylaldehyde product from the first reaction step is then mixed with dimethylpyrrole (FIG. 12A) or diphenylpyrrole (FIG. 12B) at a 1:2 molar ratio in dichloromethane (DCM) with a 50 microliter (μL) aliquot of trifluoroacetic acid (TFA) and stirred for 3 hours at room temperature under inert conditions. This is followed by the addition of 1 molar equivalent of p-chloranil with additional stirring at room temperature for 1 hour. Subsequent addition of excess trimethylamine (TEA) followed by excess boron trifluoride etherate (BF₃OEt₂) is added via syringe and allowed to stir for 1 hour. Isolated product from column chromatography purification techniques yields the BODIPY-based chromophore (shown specifically as N-phenyl-aza-15-crown-5-ether BODIPY and N-phenyl-aza-15-crown-5-ether tetraphenyl BODIPY for sodium ion sensing (Na⁺) in FIGS. 12A and 12B, respectively) for metal electrolyte sensing.

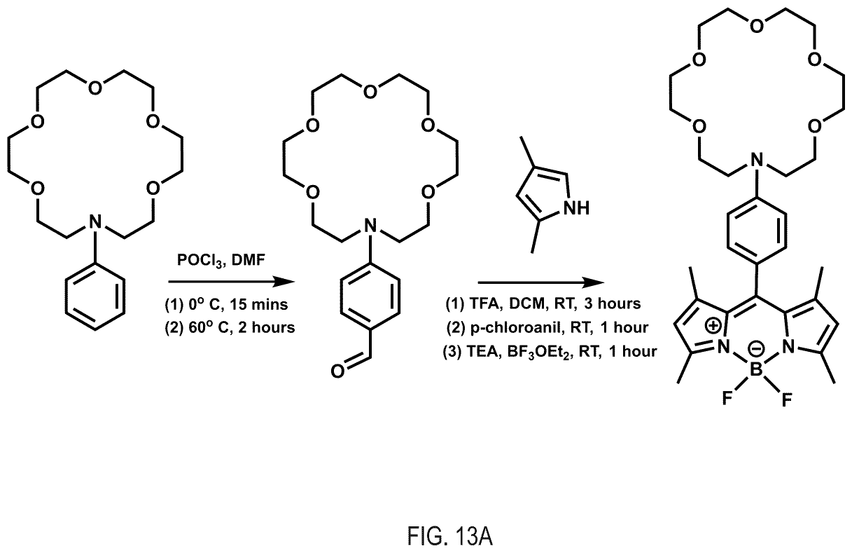

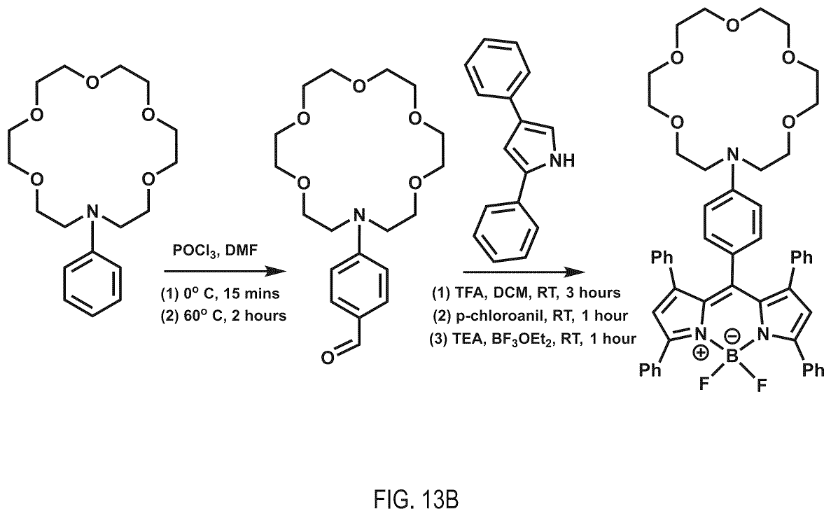

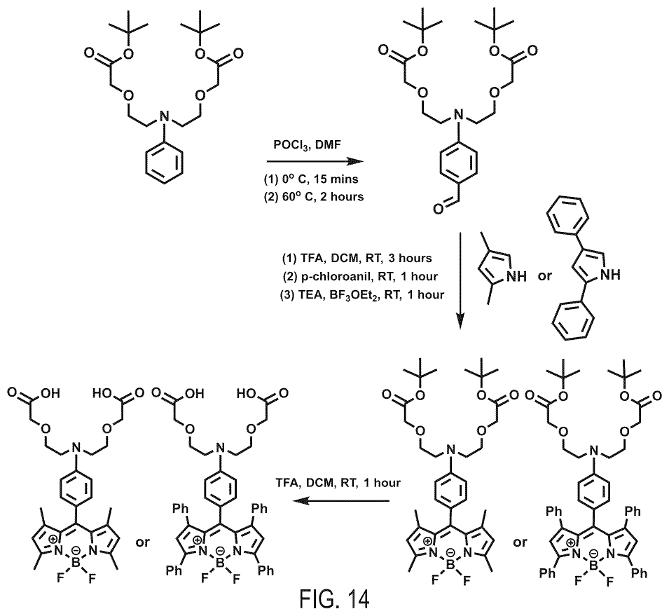

Referring next to FIGS. 13A, 13B, 14, 15, 16A and 16B, the synthetic approach and molecular structure for other metal electrolytes (specifically potassium, K⁺ in the form of N-phenyl-aza-18-crown-6-ether BODIPY in FIG. 13A and N-phenyl-aza-18-crown-6-ether tetraphenyl BODIPY in FIG. 13B. and calcium, Ca²⁺ in the form of N-phenylazabis(ethoxy)acetic acid BODIPY and N-phenylazabis(ethoxy)acetic acid tetraphenyl BODIPY in FIG. 14) are shown. The synthetic approach to synthesize these chromophores follow similar synthetic pathway to the sodium sensing analogue shown in FIGS. 12A and 12B with the exception of an additional acidification step with treatment of trifluoroacetic acid (TFA) in dichloromethane (DCM) for 1 hour at room temperature for the Ca²⁺ sensing analogue.

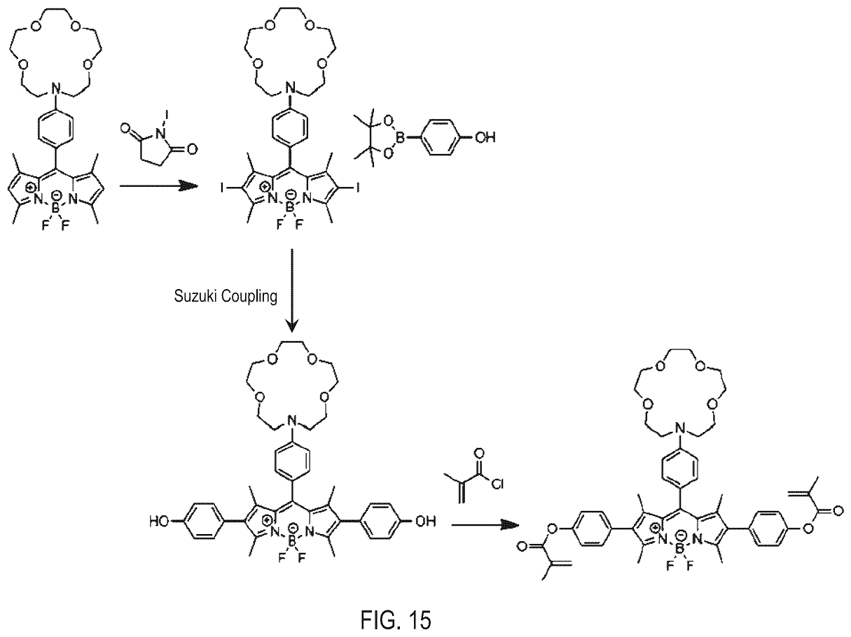

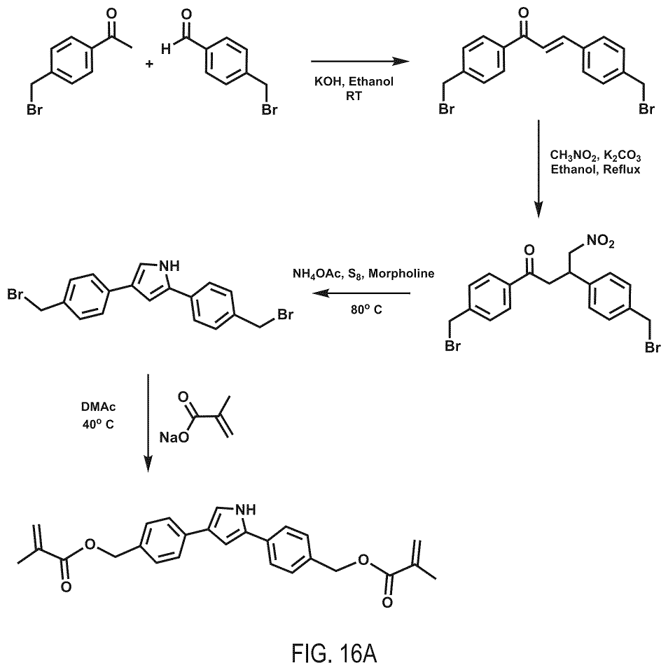

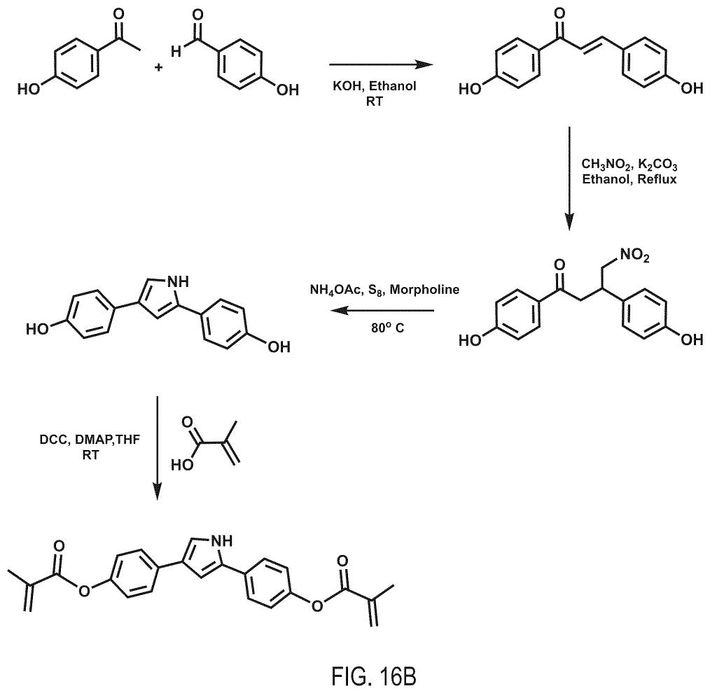

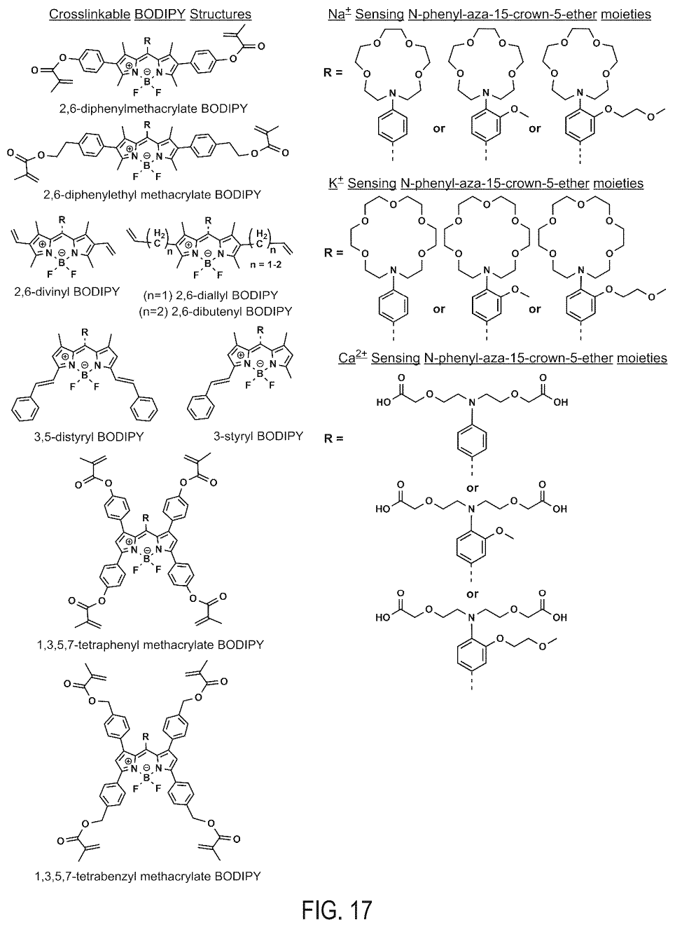

As previously noted, FIG. 6 shows the mechanism used for sodium ion sensing; however, it does not in and of itself provide a full explanation of how a chemically-bound formulation using one of the chromophores disclosed herein is achieved. Referring next to FIG. 15, an example of a sodium ion sensing synthesizable chromophore is shown. This chromophore is capable of underdoing a crosslinking step to chemically bind to a biocompatible polymer. The figure depicts a synthetic pathway to provide appropriate functionalities for chemical attachment to a hydrogel or other coating. The first step requires an iodination step through use of an iodine source (such as N-iodosuccinimide). Second, a cross coupling reaction, such as a Suzuki coupling reaction (such as depicted with particularity in FIG. 15), takes place to add a phenol functionality that can then react with a methacryloyl chloride for appropriate acrylate functionality for crosslinking into acrylate-based biocompatible polymers. Methacrylate functionality can also be afforded via appropriate para functionalization of diphenylpyrrole BODIPY precursors (as depicted with particularity in FIGS. 16A and B). In some embodiments, this may be generally achieved through a four-step synthetic pathway: (1) enol condensation of appropriately para functionalized benzaldehyde with appropriately para functionalized acetophenone, (2) Michael addition of nitromethane, (3) heterocyclic ring closure to yield diphenyl pyrrole, and finally (4) nucleophilic substitution reaction with sodium methacrylate to bromoalkene functionality or DCC coupling of the phenol to methacrylic acid. All para-functionalized benzaldehyde and acetophenones are commercially available. Additionally, crosslinking moieties can also be afforded by Knoevenagle condensation with phenyl aldehydes to afford crosslinkable styryl functionalities without the need of halogenation and cross coupling chemistry. By crosslinking the chromophore to a biocompatible polymer, leaching of the chromophore out of the biocompatible polymer is prevented. It is desirable to prevent chromophore leaching from a biocompatibility perspective because a leached chromophore would be free to migrate within the tissue of the individual. Additionally, if a chromophore is leached from the coating, the strength of the received light would decrease accordingly, potentially introducing error to the reading.

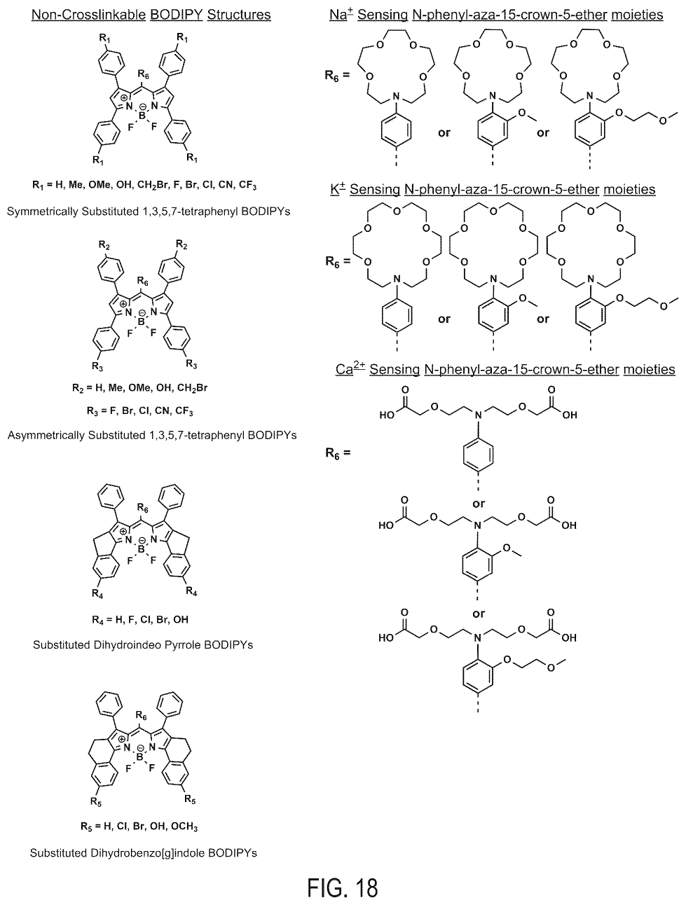

Referring next to FIG. 17, derivations of crosslinking moieties can be afforded using similar synthetic chemistry methodologies. As shown, a variety of metal ion sensing moieties can be combined with any of the BODIPY structures that have added functionality to enable crosslinking to acrylate-based polymers. Synthesizable sodium ion sensing chromophores that can be crosslinked to acrylate-based polymers include N-phenyl-aza-15-crown-5-ether 2,6-diphenylmethacrylate BODIPY, N-phenyl-aza-15-crown-5-ether 2,6-diphenylethyl methacrylate BODIPY, N-phenyl-aza-15-crown-5-ether 2,6-divinyl BODIPY, N-phenyl-aza-15-crown-5-ether 2,6-diallyl BODIPY, N-phenyl-aza-15-crown-5-ether 2,6-dibutenyl BODIPY, N-phenyl-aza-15-crown-5-ether 3,5-distyryl BODIPY, N-phenyl-aza-15-crown-5-ether 3-styryl BODIPY, N-phenyl-aza-15-crown-5-ether 1,3,5,7-tetraphenyl methacrylate BODIPY, N-phenyl-aza-15-crown-5-ether 1,3,5,7-tetrabenzyl methacrylate BODPIY, o-methoxy-N-phenyl-aza-15-crown-5-ether 2,6-diphenylmethacrylate BODIPY, o-methoxy-N-phenyl-aza-15-crown-5-ether 2,6-diphenylethyl methacrylate BODIPY, o-methoxy-N-phenyl-aza-15-crown-5-ether 2,6-divinyl BODIPY, o-methoxy-N-phenyl-aza-15-crown-5-ether 2,6-diallyl BODIPY, o-methoxy-N-phenyl-aza-15-crown-5-ether 2,6-dibutenyl BODIPY, o-methoxy-N-phenyl-aza-15-crown-5-ether 3,5-distyryl BODIPY, o-methoxy-N-phenyl-aza-15-crown-5-ether 3-styryl BODIPY, o-methoxy-N-phenyl-aza-15-crown-5-ether 1,3,5,7-tetraphenyl methacrylate BODIPY, o-methoxy-N-phenyl-aza-15-crown-5-ether 1,3,5,7-tetrabenzyl methacrylate BODPIY, o-methoxyethoxy-N-phenyl-aza-15-crown-5-ether 2,6-diphenylmethacrylate BODIPY, o-methoxyethoxy-N-phenyl-aza-15-crown-5-ether 2,6-diphenylethyl methacrylate BODIPY, o-methoxyethoxy-N-phenyl-aza-15-crown-5-ether 2,6-divinyl BODIPY, o-methoxyethoxy-N-phenyl-aza-15-crown-5-ether 2,6-diallyl BODIPY, o-methoxyethoxy-N-phenyl-aza-15-crown-5-ether 2,6-dibutenyl BODIPY, o-methoxyethoxy-N-phenyl-aza-15-crown-5-ether 3,5-distyryl BODIPY, o-methoxyethoxy-N-phenyl-aza-15-crown-5-ether 3-styryl BODIPY, o-methoxyethoxy-N-phenyl-aza-15-crown-5-ether 1,3,5,7-tetraphenyl methacrylate BODIPY, and o-methoxyethoxy-N-phenyl-aza-15-crown-5-ether 1,3,5,7-tetrabenzyl methacrylate BODPIY. Furthermore, synthesizable potassium ion sensing chromophores that can be crosslinked to acrylate-based polymers include N-phenyl-aza-18-crown-6-ether 2,6-diphenylmethacrylate BODIPY, N-phenyl-aza-18-crown-6-ether 2,6-diphenylethyl methacrylate BODIPY, N-phenyl-aza-18-crown-6-ether 2,6-divinyl BODIPY, N-phenyl-aza-18-crown-6-ether 2,6-diallyl BODIPY, N-phenyl-aza-18-crown-6-ether 2,6-dibutenyl BODIPY, N-phenyl-aza-18-crown-6-ether 3,5-distyryl BODIPY, N-phenyl-aza-18-crown-6-ether 3-styryl BODIPY, N-phenyl-aza-18-crown-6-ether 1,3,5,7-tetraphenyl methacrylate BODIPY, N-phenyl-aza-18-crown-6-ether 1,3,5,7-tetrabenzyl methacrylate BODPIY, o-methoxy-N-phenyl-aza-18-crown-6-ether 2,6-diphenylmethacrylate BODIPY, o-methoxy-N-phenyl-aza-18-crown-6-ether 2,6-diphenylethyl methacrylate BODIPY, o-methoxy-N-phenyl-aza-18-crown-6-ether 2,6-divinyl BODIPY, o-methoxy-N-phenyl-aza-18-crown-6-ether 2,6-diallyl BODIPY, o-methoxy-N-phenyl-aza-18-crown-6-ether 2,6-dibutenyl BODIPY, o-methoxy-N-phenyl-aza-18-crown-6-ether 3,5-distyryl BODIPY, o-methoxy-N-phenyl-aza-18-crown-6-ether 3-styryl BODIPY, o-methoxy-N-phenyl-aza-18-crown-6-ether 1,3,5,7-tetraphenyl methacrylate BODIPY, o-methoxy-N-phenyl-aza-18-crown-6-ether 1,3,5,7-tetrabenzyl methacrylate BODPIY, o-methoxyethoxy-N-phenyl-aza-18-crown-6-ether 2,6-diphenylmethacrylate BODIPY, o-methoxyethoxy-N-phenyl-aza-18-crown-6-ether 2,6-diphenylethyl methacrylate BODIPY, o-methoxyethoxy-N-phenyl-aza-18-crown-6-ether 2,6-divinyl BODIPY, o-methoxyethoxy-N-phenyl-aza-18-crown-6-ether 2,6-diallyl BODIPY, o-methoxyethoxy-N-phenyl-aza-18-crown-6-ether 2,6-dibutenyl BODIPY, o-methoxyethoxy-N-phenyl-aza-18-crown-6-ether 3,5-distyryl BODIPY, o-methoxyethoxy-N-phenyl-aza-18-crown-6-ether 3-styryl BODIPY, o-methoxyethoxy-N-phenyl-aza-18-crown-6-ether 1,3,5,7-tetraphenyl methacrylate BODIPY, and o-methoxyethoxy-N-phenyl-aza-18-crown-6-ether 1,3,5,7-tetrabenzyl methacrylate BODPIY. Synthesizable calcium ion sensing chromophores that can be crosslinked to acrylate-based polymers includes N-phenylazabis(ethoxy)acetic acid 2,6-diphenylmethacrylate BODIPY, N-phenylazabis(ethoxy)acetic acid 2,6-diphenylethyl methacrylate BODIPY, N-phenylazabis(ethoxy)acetic acid 2,6-divinyl BODIPY, N-phenylazabis(ethoxy)acetic acid 2,6-diallyl BODIPY, N-phenylazabis(ethoxy)acetic acid 2,6-dibutenyl BODIPY, N-phenylazabis(ethoxy)acetic acid 3,5-distyryl BODIPY, N-phenylazabis(ethoxy)acetic acid 3-styryl BODIPY, N-phenylazabis(ethoxy)acetic acid 1,3,5,7-tetraphenyl methacrylate BODIPY, N-phenylazabis(ethoxy)acetic acid 1,3,5,7-tetrabenzyl methacrylate BODPIY, o-methoxy-N-phenylazabis(ethoxy)acetic acid 2,6-diphenylmethacrylate BODIPY, o-methoxy-N-phenylazabis(ethoxy)acetic acid 2,6-diphenylethyl methacrylate BODIPY, o-methoxy-N-phenylazabis(ethoxy)acetic acid 2,6-divinyl BODIPY, o-methoxy-N-phenylazabis(ethoxy)acetic acid 2,6-diallyl BODIPY, o-methoxy-N-phenylazabis(ethoxy)acetic acid 2,6-dibutenyl BODIPY, o-methoxy-N-phenylazabis(ethoxy)acetic acid 3,5-distyryl BODIPY, o-methoxy-N-phenylazabis(ethoxy)acetic acid 3-styryl BODIPY, o-methoxy-N-phenylazabis(ethoxy)acetic acid 1,3,5,7-tetraphenyl methacrylate BODIPY, o-methoxy-N-phenylazabis(ethoxy)acetic acid 1,3,5,7-tetrabenzyl methacrylate BODPIY, o-methoxyethoxy-N-phenylazabis(ethoxy)acetic acid 2,6-diphenylmethacrylate BODIPY, o-methoxyethoxy-N-phenylazabis(ethoxy)acetic acid 2,6-diphenylethyl methacrylate BODIPY, o-methoxyethoxy-N-phenylazabis(ethoxy)acetic acid 2,6-divinyl BODIPY, o-methoxyethoxy-N-phenylazabis(ethoxy)acetic acid 2,6-diallyl BODIPY, o-methoxyethoxy-N-phenylazabis(ethoxy)acetic acid 2,6-dibutenyl BODIPY, o-methoxyethoxy-N-phenylazabis(ethoxy)acetic acid 3,5-distyryl BODIPY, o-methoxyethoxy-N-phenylazabis(ethoxy)acetic acid 3-styryl BODIPY, o-methoxyethoxy-N-phenylazabis(ethoxy)acetic acid 1,3,5,7-tetraphenyl methacrylate BODIPY, and o-methoxyethoxy-N-phenylazabis(ethoxy)acetic acid 1,3,5,7-tetrabenzyl methacrylate BODPIY. This list is not meant to be exhaustive and any reasonable structure attainable from these chemistries is within the spirit and scope of this disclosure.

Referring next to FIG. 18, structures of metal ion sensing moieties can be combined with any of the BODIPY structures to form analyte-sensing chromophores that do not contain functionality that enables them to be crosslinked to acrylate-based polymers. Synthesizable sodium ion sensing chromophores that do not contain crosslinking functionality includes N-phenyl-aza-15-crown-5-ether 1,3,5,7-tetraphenyl BODIPY, o-methoxy-N-phenyl-aza-15-crown-5-ether 1,3,5,7-tetraphenyl BODIPY, and o-methoxyethoxy-N-phenyl-aza-15-crown-5-ether 1,3,5,7-tetraphenyl BODIPY that can be symmetrically tetra-substituted in the paraphenyl position where the R₁ group depicted in FIG. 18 may be H, Me, OMe, OH, CH₂Br, F, Br, CI, CN or CF₃ or asymmetrically substituted in the in the paraphenyl position where the R₂ depicted in FIG. 18 may be H, Me, OMe, OH, or CH₂Br, and R₃ may be F, Br, CI, CN or CF₃. Other sodium ion sensing chromophores that do not contain crosslinking functionality includes tetra-substituted N-phenyl-aza-15-crown-5-ether dihydroindeo pyrrole BODIPY, o-methoxy-N-phenyl-aza-15-crown-5-ether dihydroindeo pyrrole BODIPY, and o-methoxyethoxy-N-phenyl-aza-15-crown-5-ether dihydroindeo pyrrole BODIPY where the R₄ group may be H, F, Cl, Br or OH and N-phenyl-aza-15-crown-5-ether dihydrobenzo[g]indole pyrrole BODIPY, o-methoxy-N-phenyl-aza-15-crown-5-ether dihydrobenzo[g]indole pyrrole BODIPY, and o-methoxyethoxy-N-phenyl-aza-15-crown-5-ether dihydrobenzo[g]indole pyrrole BODIPY where R₅ group may be H, Cl, Br, OH, or OCH₃. Synthesizable potassium ion sensing chromophores that do not contain crosslinking functionality includes N-phenyl-aza-18-crown-6-ether 1,3,5,7-tetraphenyl BODIPY, o-methoxy-N-phenyl-aza-18-crown-6-ether 1,3,5,7-tetraphenyl BODIPY, and o-methoxyethoxy-N-phenyl-aza-18-crown-6-ether 1,3,5,7-tetraphenyl BODIPY that can be symmetrically tetra-substituted in the paraphenyl position where the R₁ group depicted in FIG. 18 may be H, Me, OMe, OH, CH₂Br, F, Br, Cl, CN or CF₃ or asymmetrically substituted in the in the paraphenyl position where the R₂ depicted in FIG. 18 may be H, Me, OMe, OH, or CH₂Br, and R₃ may be F, Br, Cl, CN or CF₃. Other potassium ion sensing chromophores that do not contain crosslinking functionality includes tetra-substituted N-phenyl-aza-18-crown-6-ether dihydroindeo pyrrole BODIPY, o-methoxy-N-phenyl-aza-18-crown-6-ether dihydroindeo pyrrole BODIPY, and o-methoxyethoxy-N-phenyl-aza-18-crown-6-ether dihydroindeo pyrrole BODIPY where R₄ group may be H, F, Cl, Br or OH and N-phenyl-aza-18-crown-6-ether dihydrobenzo[g]indole pyrrole BODIPY, o-methoxy-N-phenyl-aza-18-crown-6-ether dihydrobenzo[g]indole pyrrole BODIPY, and o-methoxyethoxy-N-phenyl-aza-18-crown-6-ether dihydrobenzo[g]indole pyrrole BODIPY where R₅ group may be H, Cl, Br, OH or OCH₃. Synthesizable calcium ion sensing chromophores that do not contain crosslinking functionality includes N-phenylazabis(ethoxy)acetic acid 1,3,5,7-tetraphenyl BODIPY, o-methoxy-N-phenylazabis(ethoxy)acetic acid 1,3,5,7-tetraphenyl BODIPY, and o-methoxyethoxy-N-phenylazabis(ethoxy)acetic acid 1,3,5,7-tetraphenyl BODIPY that can be symmetrically tetra-substituted in the paraphenyl position where the R₁ group depicted in FIG. 18 may be H, Me, OMe, OH, CH₂Br, F, Br, Cl, CN or CF₃ or asymmetrically substituted in the in the paraphenyl position where the R₂ depicted in FIG. 18 may be H, Me, OMe, OH, or CH₂Br, and R₃ may be F, Br, Cl, CN or CF₃. Other calcium ion sensing chromophores that do not contain crosslinking functionality includes tetra-substituted N-phenylazabis(ethoxy)acetic acid dihydroindeo pyrrole BODIPY, o-methoxy-N-phenylazabis(ethoxy)acetic acid dihydroindeo pyrrole BODIPY, and o-methoxyethoxy-N-phenylazabis(ethoxy)acetic acid dihydroindeo pyrrole BODIPY where R₄ group may be H, F, Cl, Br, or OH and N-phenylazabis(ethoxy)acetic acid dihydrobenzo[g]indole pyrrole BODIPY, o-methoxy-N-phenylazabis(ethoxy)acetic acid dihydrobenzo[g]indole pyrrole BODIPY, and o-methoxyethoxy-N-phenylazabis(ethoxy)acetic acid dihydrobenzo[g]indole pyrrole BODIPY where R₅ group may be H, Cl, Br, OH, or OCH₃. All of the symmetric/asymmetric non-crosslinkable 1,3,5,7-tetraphenyl BODIPYs precursors are synthesized using the same chemistry previously described: (1) enol condensation of appropriately para functionalized benzaldehyde with appropriately para functionalized acetophenone, (2) Michael addition, (3) ring closure to the diphenyl pyrrole. All para-functionalized benzaldehyde and acetophenones are commercially available. N-heterocyclic pyrrole precursor for dihydroindeno pyrrole BODIPYS can be synthesized via lithium diisopropylamide mediated reaction of the appropriate substituted 1-indanone with appropriate substituted 3-phenyl-2H-azirene. Substituted 1-indadones are commercially available while 3-phenyl-2H-azirenes derivatives are synthesized from styrene. Pyrrole precursors for substituted dihydrobenzo[g]indole BODIPYS are afforded in the same synthetic pathway using 1-tetralone with 3-phenyl-2H-azirene. It will be appreciated that the non-exhaustive nature of the list of chromophores mentioned in FIG. 17 is also applicable—mutatis mutandis—to the moieties of FIG. 18.

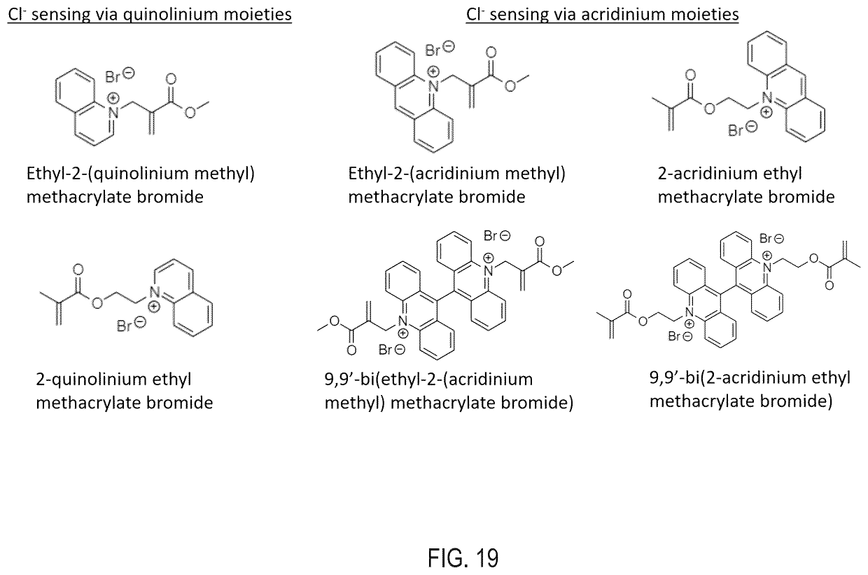

Referring next to FIG. 19, structures for synthesizable quinolinium- and acridinium-based chromophores are shown. Ethyl-2-(quinolinium methyl) methacrylate bromide, 2-quinolinium ethyl methacrylate bromide, ethyl-2-(acridinium methyl) methacrylate bromide, and 2-acridinium ethyl methacrylate bromide, 9,9′-bi (ethyl-2-(acridinium methyl) methacrylate bromide), and 9,9′-bi (2-acridinium ethyl methacrylate bromide) are designed for sensing chloride ions and could be chemically bound to an acrylate-based polymer. It will be appreciated that the non-exhaustive nature of the list of chromophores mentioned in FIGS. 17 and 18 is also applicable—mutatis mutandis—to the moieties of FIG. 19.

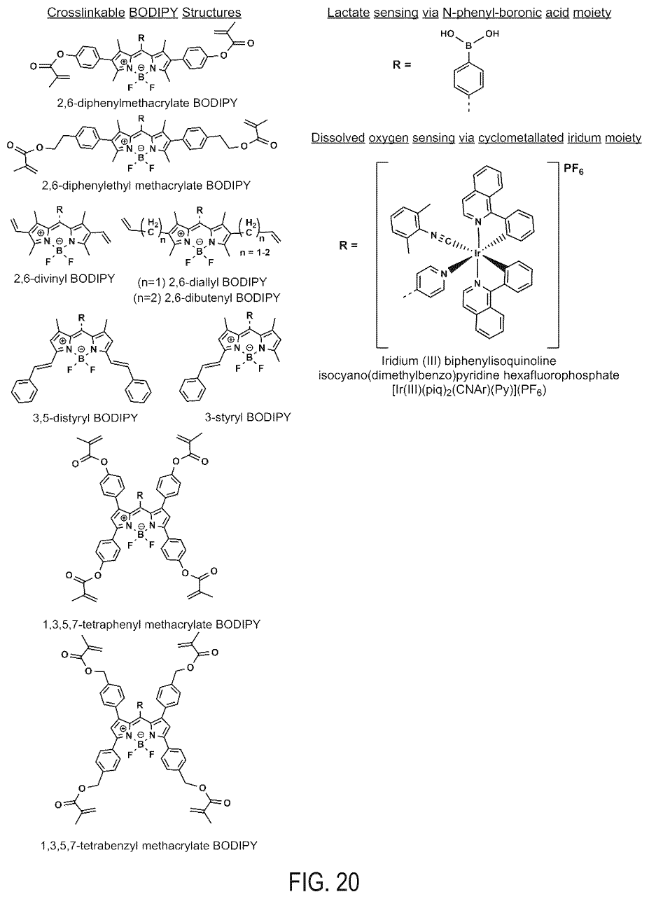

Referring next to FIG. 20, structures for synthesizable N-phenyl-boronic acid BODIPYs and synthesizable cyclometallated iridium(III) BODIPYs are shown. The N-phenyl-boronic acid BODIPYs comprise N-phenyl-boronic acid 2,6-diphenylmethacrylate BODIPY, N-phenyl-boronic acid 2,6-diphenylethyl methacrylate BODIPY, N-phenyl-boronic acid 2,6-divinyl BODIPY, N-phenyl-boronic acid 2,6-diallyl BODIPY, N-phenyl-boronic acid 2,6-dibutenyl BODIPY, N-phenyl-boronic acid 3,5-distyryl BODIPY, and N-phenyl-boronic acid 3-styryl BODIPY, N-phenyl-boronic acid 1,3,5,7-tetraphenyl methacrylate BODIPY, and N-phenyl-boronic acid 1,3,5,7-tetrabenzyl methacrylate BODPIY. These N-phenyl-boronic acid BODIPY chromophores could be used to sense lactate via changes in emission intensity and contain the appropriate functionality such that they could be crosslinked to acrylate-based polymers. The cyclometallated iridium(III) BODIPYs comprise [(Iridium (III) biphenylisoquinoline isocyano(dimethylbenzo) pyridine) (2,6-diphenylmethacrylate BODIPY)](hexafluorophosphate), [(iridium(III) biphenylisoquinoline isocyano(dimethylbenzo) pyridine) (2,6-diphenylethyl methacrylate BODIPY)](hexafluorophosphate), [(Iridium (III) biphenylisoquinoline isocyano(dimethylbenzo) pyridine) (2,6-divinyl BODIPY)](hexafluorophosphate), [(iridium(III) biphenylisoquinoline isocyano(dimethylbenzo) pyridine) (2,6-diallyl BODIPY)](hexafluorophosphate), [(iridium(III) biphenylisoquinoline isocyano(dimethylbenzo) pyridine) (2,6-dibutenyl BODIPY)](hexafluorophosphate), [(Iridium (III) biphenylisoquinoline isocyano(dimethylbenzo) pyridine) (3,5-distyryl BODIPY)](hexafluorophosphate), [(iridium(III) biphenylisoquinoline isocyano(dimethylbenzo) pyridine) (3-styryl BODIPY)](hexafluorophosphate)], [(iridium(III) biphenylisoquinoline isocyano(dimethylbenzo) pyridine) (1,3,5,7-tetraphenyl methacrylate BODIPY)](hexafluorophosphate), and [(iridium(III) biphenylisoquinoline isocyano(dimethylbenzo) pyridine) (1,3,5,7-tetrabenzyl methacrylate BODPIY)](hexafluorophosphate). These cyclometallated iridium(III) BODIPY chromophores should be dual luminescent with an oxygen-insensitive fluorescence emission and an oxygen-sensitive phosphorescence emission. In this manner, they could be used as ratiometric intensity dissolved oxygen sensors. These chromophores also contain the appropriate functionality such that they could be crosslinked to acrylate-based polymers. It will be appreciated that the non-exhaustive nature of the list of chromophores mentioned in FIGS. 17 through 19 is also applicable—mutatis mutandis—to the moieties of FIG. 20.

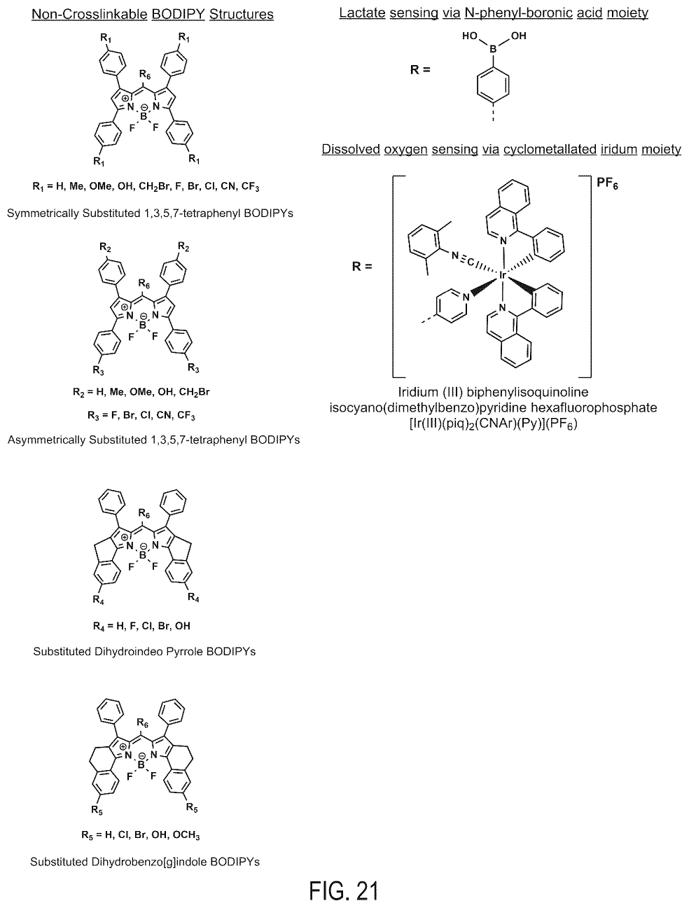

Referring next to FIG. 21, structures for other synthesizable N-phenyl-boronic acid BODIPYs and synthesizable cyclometallated iridium(III) BODIPYs are shown. It is noted that unlike FIG. 20, the structures shown in FIG. 21 do not contain functionality that enables them to be crosslinked to acrylate-based polymers. The N-phenyl-boronic acid BODIPYs that do not contain crosslinking functionality includes N-phenyl-boronic acid 1,3,5,7-tetraphenyl BODIPY that can be symmetrically tetra-substituted in the paraphenyl position where the R₁ group depicted in FIG. 21 may be H, Me, OMe, OH, CH₂Br, F, Br, Cl, CN or CF₃ or asymmetrically substituted in the in the paraphenyl position where the R₂ depicted in FIG. 21 may be H, Me, OMe, OH, or CH₂Br, and R₃ may be F, Br, Cl, CN or CF₃. Other N-phenyl-boronic acid BODIPYs that do not contain crosslinking functionality includes N-phenyl-boronic acid dihydroindeo pyrrole BODIPY where R₄ group may be H, F, Cl, Br, or OH and N-phenyl-boronic acid dihydrobenzo[g]indole pyrrole BODIPY where R₅ group may be H, Cl, Br, OH, or OCH₃. These N-phenyl-boronic acid BODIPY chromophores could be used to sense lactate via changes in emission intensity in polymeric hosts. The cyclometallated iridium(III) BODIPYs comprise that do not contain crosslinking functionality includes [(iridium(III) biphenylisoquinoline isocyano(dimethylbenzo) pyridine) (1,3,5,7-tetraphenyl BODIPY)](hexafluorophosphate) that can be symmetrically tetra-substituted in the paraphenyl position where the R₁ group depicted in FIG. 21 may be H, Me, OMe, OH, CH₂Br, F, Br, Cl, CN or CF₃ or asymmetrically substituted in the in the paraphenyl position where the R₂ depicted in FIG. 21 may be H, Me, OMe, OH, or CH₂Br, and R₃ may be F, Br, CI, CN or CF₃. Other cyclometallated iridium(III) BODIPYs that do not contain crosslinking functionality includes [(iridium(III) biphenylisoquinoline isocyano(dimethylbenzo) pyridine) (dihydroindeo pyrrole BODIPY)](hexafluorophosphate) where R₄ group may be H, F, Cl, Br, or OH and [(iridium(III) biphenylisoquinoline dihydrobenzo[g]indole pyrrole BODIPY)](hexafluorophosphate) where R₅ group may be H, Cl, Br, OH or OCH₃. It will be appreciated that the non-exhaustive nature of the list of chromophores mentioned in FIGS. 17 through 20 is also applicable—mutatis mutandis—to the moieties of FIG. 21.

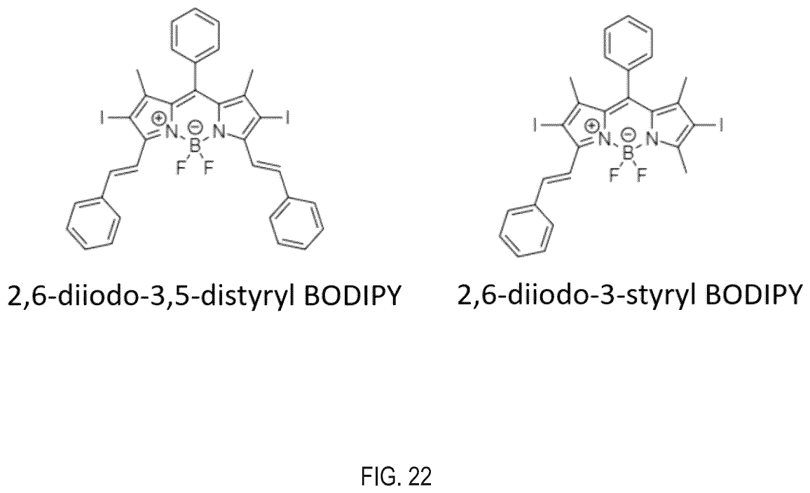

Referring next to FIG. 22, other synthesizable, ratiometric dissolved oxygen sensor chromophores are depicted in the form of iodo-BODIPYs. These chromophores can be crosslinked to acrylate-based polymers. The depicted chromophores comprise 2,6-diiodo-3,5-distyryl BODIPY and 2,6-diiodo-3-styryl BODIPY. It will be appreciated that the non-exhaustive nature of the list of chromophores mentioned in FIGS. 17 through 21 is also applicable—mutatis mutandis—to the moieties of FIG. 22.

Referring next to FIGS. 23 through 27, the analyte-sensing system 100 of FIGS. 1 and 2 is extended to allow enhanced detection through a multiplex sensing system 1000 that provides added flexibility to the sensor functionality of the individual microneedles. In some embodiments, an individual microneedle may include both an analyte-sensing chromophore and a reference (that is to say, non-analyte sensing) chromophore. Likewise, other individual microneedles may only include the analyte sensing chromophore while still others only include the reference chromophore. Likewise, other individual microneedles may include no chromophores. Numerous variations and combinations of all such individual microneedles are thus deemed to be within the spirit and scope of the present disclosure. In addition to the various microneedle configurations, the multiplex sensing system 1000 may include optical equipment to perform one or more of analyzing, storing and presenting results based on the sensed analytes. In some embodiments, the multiplex sensing system 1000 is made up of a reusable portion (shown generally as an optical processing system 1100 that in turn may be an assembly of numerous pieces of equipment that will be discussed in more detail as follows) and a disposable portion in the form of an optical sensor array 1200 that is made of relatively inexpensive components. By this construction, the optical sensor array 1200 may have numerous microneedle-chromophore combinations such that one or more of the microneedle-chromophore combinations have the ability to be analyte sensing while one or more of the microneedle-chromophore combinations are used as a reference in that it or they are not analyte sensing.

Referring with particularity to FIG. 23, the optical sensor array 1200 pairs (i.e., it is communicatively coupled) with a detector 1400 to be able to report on the concentrations of multiple physiological analytes present in the ISF 25 in a minimally invasive manner. In some embodiments, the optical sensor array 1200 includes a substrate 1210 and numerous optical sensors (shown presently in the form of optically transparent microneedles 1220). In some embodiments, one or more of the microneedles 1220 may include a microlens (not shown) in order to optimize light collection. In addition, the optical sensor array 1200 either includes or is otherwise coupled to a corresponding number of optical fibers 1230 and a coupling device that in some embodiments is a linear fiber array 1240, presently shown in both a side view and an edge-on fiber-receiving view. In some embodiments, the optical fibers 1230 may be integrated with the optical sensor array 1200 to form an assembly 1250. In other embodiments, optical fibers 1230 may be a separate component from the optical sensor array 1200. In other words, the optical fibers 1230 may be packaged either separately or with the linear fiber array 1240, optical processing system 1100 or other parts of the multiplex sensing system 1000. Although optical fibers 1230 are presently shown, this configuration could alternatively use a fused fiber waveguide or semiconductor photonic waveguide, as will be discussed in more detail below. Likewise, the linear fiber array 1240 may assume a more general shape (i.e., non-linear, such as circular); both forms being within the spirit and scope of the present disclosure. In some embodiments, the multiplex sensing system 1000 may include more than one coupling device which are not shown in FIG. 23 for simplicity.

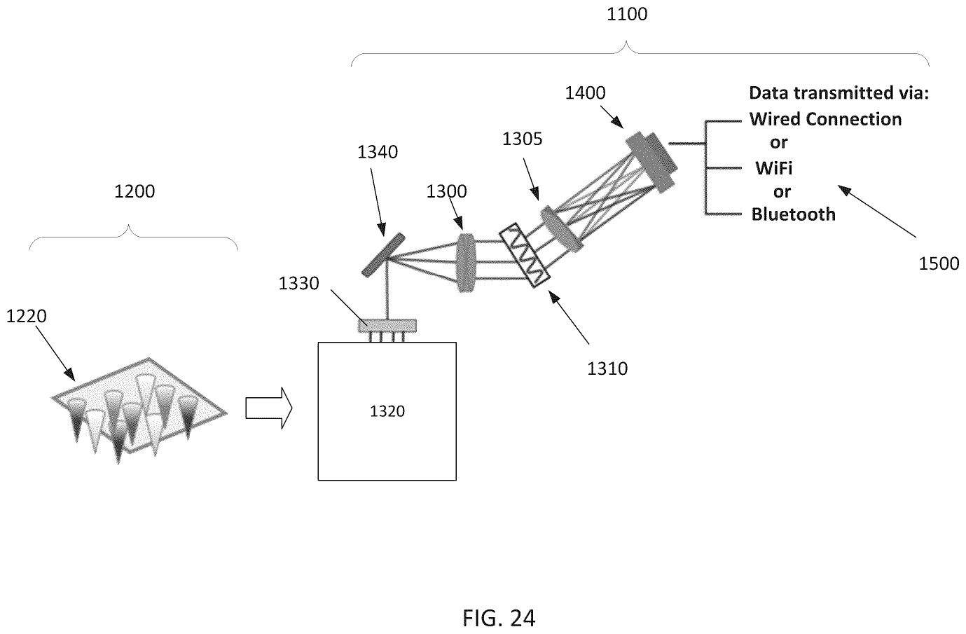

As discussed in connection with the optical fibers 1230, the linear fiber array 1240 may be integrated with the optical sensor array 1200 to form an assembly 1250. However, this is not limiting, and the linear fiber array 1240 and the optical sensor array 1200 may be separate components. In other words, the linear fiber array 1240 and the optical sensor array 1200 may be packaged either separately or with one or more of the optical fibers 1230, the optical processing system 1100 or other parts of the multiplex sensing system 1000. It will be appreciated that all variants are within the scope and spirit of the present disclosure. In some embodiments, a key benefit of assembly 1250 is that it can serve as a prepackaged module where each individual microneedle 1220 (or other sensor, depending on the configuration of the optical sensor array 1200) defines a dedicated signal path. Regardless of the form, a light signal emanating from a single microneedle 1220 has a dedicated signal path in order to be conveyed individually—rather than grouped together—to the optical processing system 1100 in a manner to conserve each signal's unique optical attributes. It will be appreciated that although the linear fiber array 1240 acts as a dedicated one-to-one coupling between each optical fiber 1230 and one or more downstream components of the optical processing system 1100, other forms of coupling device may be used, including those that ensure signal continuity all the way from the microneedles 1220 and the one or more collimating lens 1300, including the various optical fibers 1230 or one or more waveguides 1320 as will be discussed in more detail below in connection with FIG. 24. It will be appreciated that the number of components that make up such coupling device (e.g., coupling device 1200) will be apparent from the context. Likewise, the coupling device may be structured to establish a so-called quick-connect (which may include snap-fit, plug-and-play or other related connection configurations, where all such variants are within the scope of the present disclosure). For example, although not shown, a clamp, collar or related snap-on or other quick-connect fixture may be used to secure the linear fiber array 1240 (or other terminus point for one or more of the optical fibers 1230) to a collimating lens 1300, focusing lens 1305 or other component that makes up a portion of the optical processing system 1100. In some embodiments, this secure connection enabled by the coupling device includes one or both of a mechanical and an optical coupling.

In some embodiments, while the optical processing system 1100 performs some similar functions to that of the reader 400 of FIG. 1B, it will be appreciated that some of the functions performed by the microneedles 220 and the reader 400 of the device of FIG. 1B may overlap for the multi-analyte case of FIGS. 23 through 27 as a result of the connection between the optical fibers 1230 and the waveguide 1320 that will be discussed in more detail below. It will likewise be appreciated that the distinction will be apparent from the context. Within the optical processing system 1100, various components, hardware or equipment may include one or more of the collimating lens 1300, focusing lens 1305, a volume phase grating (VPG) 1310 and the detector 1400 that—although being depicted as a single component—may be an assembly made up of numerous components. In this way, the optical sensor array 1200 (and optionally the linear fiber array 1240, waveguides 1320 or other equivalent optical coupling device) that makes up the disposable portion may be packaged, sold, stored, shipped or otherwise commercialized separately from the optical processing system 1100. In some embodiments, some or all of the components that make up the multiplex sensing system 1000 may be arranged in a portable, wearable form factor to be mobile with the individual who is being monitored. By combining the optical processing system 1100 and the disposable portion in the form of an optical sensor array 1200, the multiplex sensing system 1000 can perform continuous, real-time monitoring of multiple analytes that are present in the ISF 25. As previously noted, the linear fiber array 1240 or other coupling device is formed as part of the disposable portion that is made up of the optical sensor array 1200. Alternatively, the linear fiber array 1240 or other suitable coupling device is formed as part of the optical processing system 1100; either version is within the spirit and scope of the present disclosure.

As previously noted, the individual sensors that make up the optical sensor array 1200 are configured as microneedles 1220, although it will be appreciated that other forms of sensors or related light-transmissive devices may be used to form the optical sensor array 1200. For example, sensors of different shapes (not shown) may still contain the same analyte-sensing chromophores incorporated within or coated onto a transparent material, but not be needle-shaped, such as in uses that do not require piercing of an individual's skin. By way of example and not limitation, such sensors could be used for environmental monitoring (for water or other fluids) or for monitoring analytes in extracted forms of ISF 25. It will be appreciated that regardless of whether the optical sensor array 1200 is made up of the optical microneedles 1220 or other types of optical devices, each may be configured to detect a particular analyte in a manner similar to that which is discussed herein. It will further be appreciated that all such variants of the optical sensor array 1200 are within the spirit and scope of the present disclosure, even though much of the remainder of the disclosure discusses such sensors within the specific context of a microneedle 1220.

In some embodiments, the optical sensor array 1200 is made to contain different analyte-sensing chromophores on different optical microneedles 1220. In this way, one or all of the optical microneedles 1220, the material in which the sensing is occurring (such as the hydrogel or other biocompatible polymer) as well as the one or more chromophores it contains, may define an agent. In some embodiments, this agent is either part of or otherwise cooperative with each optical microneedle 1220 to form a transdermal window. As such, the agent may be made up of an analyte-sensing chromophore (either with or without a reference chromophore) such that a corresponding microneedle-chromophore combination interacts with at least a portion of the particular analyte present in the ISF 25. Relatedly, in a more general configuration (that is to say, not just one limited to polymer-coated microneedles 1220), the agent may be defined solely by the chromophore-containing microneedle 1220. Each of the numerous microneedles 1220 includes one or more particular chromophores that are engineered to identify a particular analyte. In this way, the particular type of optical sensing microneedle 220 (that is to say, ones configured with a particular polymer coating) of FIGS. 1 and 2 may be replaced with numerous tailored optical microneedles 1220 within a single disposable optical sensor array 1200. This has the effect of making each different microneedle-chromophore combination with each optical sensor array 1200 uniquely-qualified to detect the presence of a particular analyte within the ISF 25. In other embodiments, several of the microneedles 1220 (for example, three out of nine) may be configured to target one given analyte (such as sodium ions). In this case, the remaining six microneedles 1220 would be used to target two other analytes. Having multiple distinct measurements per analyte helps provide a more reliable measurement of that given analyte. In other cases, every microneedle 1220 of the optical sensor array 1200 may be detecting another analyte (in that case, nine analytes would be targeted using the shown three-by-three array). Moreover, each optical sensor array 1200 may be tailored to particular groups of analytes, especially for those that are indicative of common or overlapping areas of medical or diagnostic interest, such as those where dissolved oxygen, electrolytes, biomarkers, neurotransmitters or other analytes of interest as discussed herein.

One way to achieve this microneedle-chromophore combination is by first fabricating the various optical microneedles 1220 and then using printing or a related form of deposition to place a polymer containing an analyte-sensing chromophore on the surface of each. By way of example, the deposition may include the use of ink jet printing. For each optical microneedle 1220, an analyte-insensitive dye may also be incorporated to act as a reference. In other embodiments, the chromophores may be incorporated into each of the optical microneedles 1220 in situ during the fabrication process of each optical microneedle 1220. For example, in a casting-based approach where material is deposited into a negative mold, the cavities that correspond to the remaining microneedles 1220 could be blocked off so that then precursor material is permitted to flow or otherwise be placed into the single negative cavity or cavities of interest. As previously noted, the optical sensor array 1200 may be configured as a stand-alone, disposable item meant for one-time use or as a part of the more comprehensive multiplex sensing system 1000. In use, the optical sensor array 1200 is inserted into the skin of an individual (using, for example, an applicator or applied manually).

As previously noted, at least some of the different optical microneedles 1220 contain different analyte-sensing chromophores to allow for sensing of a given analyte. In some embodiments, these individual optical microneedles 1220 are arranged within the optical sensor array 1200 on the substrate 1210 (shown presently as a three row by three column matrix, but more generally as an N×M matrix where “N” represents the rows and “M” represents the columns), and are coupled to a corresponding number of the optical fibers 1230 that form a signal connection between each of the optical microneedles 1220 and the linear fiber array 1240 or related coupling device. Output from such coupling device is fed through one or more collimating lenses 1300, one or more focusing lenses 1305 (which may or may not be similar in construction to the one or more collimating lenses 1300) and one or more specialized VPGs (which operates through changes of the index of refraction rather than grooved surfaces or other repetitive structures) 1310 in order to obtain a spectrum from each optical microneedle 1220 over a specified wavelength range, as well as to focus the spectral data obtained from the optical sensor array 1200 onto specified sections of the detector 1400 that corresponds to the three row by three column matrix that is representative of the optical sensor array 1200. As such, the matrix depicted by the detector 1400 is merely a representation of the data being collected thereon rather than in the form of an actual display. Thus, although not shown, it will be understood that the data that is depicted on the array of the detector 1400 could also be sent to an external device (such as a mobile phone, tablet, laptop computer, the cloud or other suitable equipment for storage, viewing or subsequent analysis and processing), if required.

Within the present disclosure, it will be appreciated that the spectrum being obtained from each optical microneedle 1220 is a spectrum that is a particular species of a larger spectrum genus. For example, if an intensity-based sensing is performed, an emission spectrum is acquired, while if colorimetric-based sensing is performed, a reflectance spectrum is obtained. It will be further appreciated that the context will dictate the precise or generalized nature of the spectrum that is being obtained. It will be appreciated that while changes that occurred in the output from the chromophores with different concentrations of the analyte may be correlated to a particular photophysical property, the mere transmission of the corresponding data is more precisely thought of as an optical signal being delivered from the chromophores. The nature of the transmitted signal versus the underlying photophysical property will be apparent from the context.

In some embodiments, the detector 1400 can be defined as a 2D complementary metal-oxide-semiconductor (CMOS), while in other embodiments as a charged-couple device (CCD) where the choice of use may be dictated by numerous considerations, including cost, ability to deploy in a wearable form factor or other integrated packaging, need for active versus passive pixels, power consumption, ability to perform real-time processing or the like. As with the coupling device, the multiplex sensing system 1000 may include more than one detector 1400. In some embodiments, the multiplex sensing system 1000 includes one or more built-in light-emitting diodes (LEDs) or laser diodes that may be used to provide an exciting light source for the optical microneedles 1220. Filters (not shown) block the excitation light from reaching the detector 1400. It will be understood that although the output is shown on the detector 1400 as an N×M array in general and a 3×3 array in particular (where N corresponds to a particular row and M to a particular column within the optical sensor array 1200), the present disclosure encompasses larger arrays, including those when N #M. The spectrum that is obtained for each individual optical microneedle 1220 may in turn be mapped out in a corresponding matrix-like manner as shown in order to represent simultaneous detection of an entirety of the N×M spectra. One or both of the raw spectral data and the estimated concentration of a given analyte is then conveyed to one or more of the aforementioned external devices over a suitable data transfer mechanism, such as a wired or wireless network 1500. Various forms of data transfer protocols may be used for the wireless version of the network 1500, such as Bluetooth or WiFi. In some embodiments, the optical processing system 1100 may itself perform its own analysis either locally in conjunction with computer system 300 or remotely with a cloud-based server, an offsite server, or other resources on the acquired data as part of a descriptive analytics approach. In other embodiments, the optical processing system 1100 may itself perform its own analysis on the acquired data as part of a data-informed predictive analytics approach. In this latter form, the optical processing system 1100 may employ machine learning so that recourse to a priori-based analysis is minimized or eliminated entirely. For example, the detection of particular analytes in the ISF 25 may be used to indicate the presence of a particular medical condition when compared against known baseline medical conditions. It will be understood that such known baseline medical conditions may be generated through a supervised, training-based model. In some embodiments, these machine learning-based approaches could potentially be used to help predict whether the changes in analyte concentration over time or the relative level of analytes compared to one another indicates a deleterious changes in a health condition. In other embodiments, such conditions may be accessed through known local or remote (i.e., external) databases, the latter of which may be accessed conventionally through internet queries, subscription-based services or the like, as well as through large language models (LLMs). In either a descriptive or predictive analytics mode of operation, the optical processing system 1100 may include any necessary equipment to establish the aforementioned wired or wireless communication.

In the example shown, the output from the optical processing system 1100 may be projected on the detector 1400 such that the data can subsequently be transmitted and viewed on another device, such as those previously discussed in conjunction with the wired or wireless network 1500. In some embodiments, this output corresponds to one or more detected analytes that may form a composite or aggregate of numerous cells or regions each of which corresponds to a particular one of the optical microneedles 1220 (or related optical sensor) and which are computed either within the optical processing system 1100 or remotely in the cloud or other suitable computational equipment. In some embodiments, the composite resembles hyperspectral imaging or multispectral imaging, either having both spatial and spectral content and where adjacent ones of the cells are either contiguous spectral bands (hyperspectral imaging) or spaced spectral bands (multispectral imaging) where images are acquired in a time-sequential manner or simultaneously, the latter of which may further include dividing all of the elements and then recombining them in one or more postprocessing steps. In some embodiments, the resulting data cube (also referred to as a three-dimensional (3D) data cube made up of one-dimensional spectral information and two-dimensional spatial information that collectively make up the spectral irradiance of a given image) projects band-sequential spatial raster images or a related signal type, response levels or the like in a pixelated manner on detector 1400.

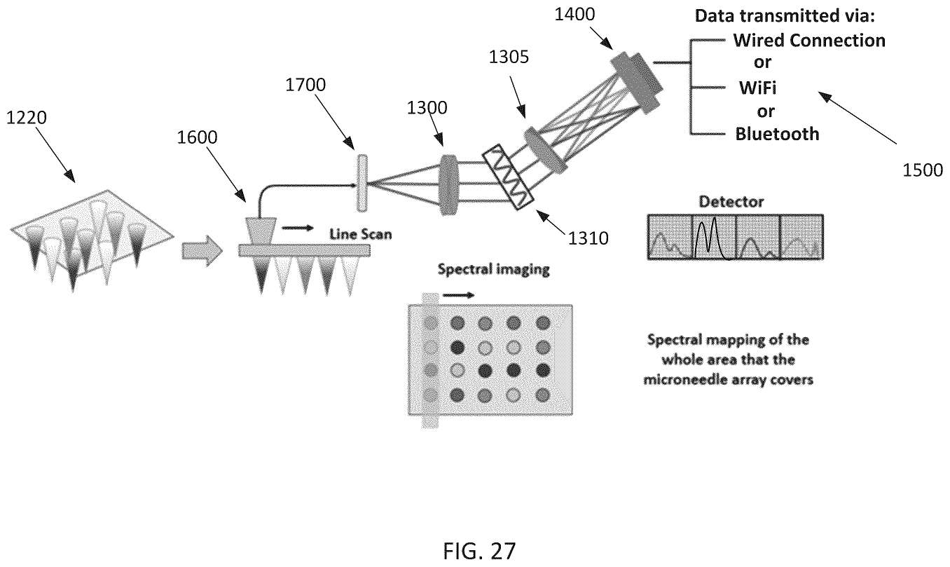

In the embodiment depicted in FIG. 23, a snapshot-based approach may be used to acquire the entire dataset during a single integration period of the detector 1400, while in other embodiments (as will be discussed in more detail in conjunction with FIG. 27) a scanning-based approach may be used to acquire the entire dataset over a certain measurement period as determined by the multiplex sensing system 1000. Thus, in contrast to conventional camera-based approaches that merely sample red-green-blue (RGB) simulations of the visible spectrum, the detector 1400 may measure each pixel (such as shown in the matrix of projected data that corresponds to the detector 1400 and which corresponds to a single spatial location within the data cube), including data that may extend beyond the visible range, thereby providing enhanced spectral resolution. By constructing the spectral irradiance into a hyperspectral data cube, the two dimensional (2D) pixels may be redefined as 3D voxels. Significantly, although only one spectral region from each individual microneedle 1220 is obtained, in the aggregate, the spectral range is enhanced since the spectra may be different for different microneedles 1220. Thus, the multiplex sensing system 1000 could allow for the simultaneous acquisition of more than one spectral region of the same acquired image from the optical sensor array 1200. This in turn allows for higher resolution imaging, as well as an enhanced ability to remove noise, clutter or other artifacts that may otherwise mask the true analyte being sensed.

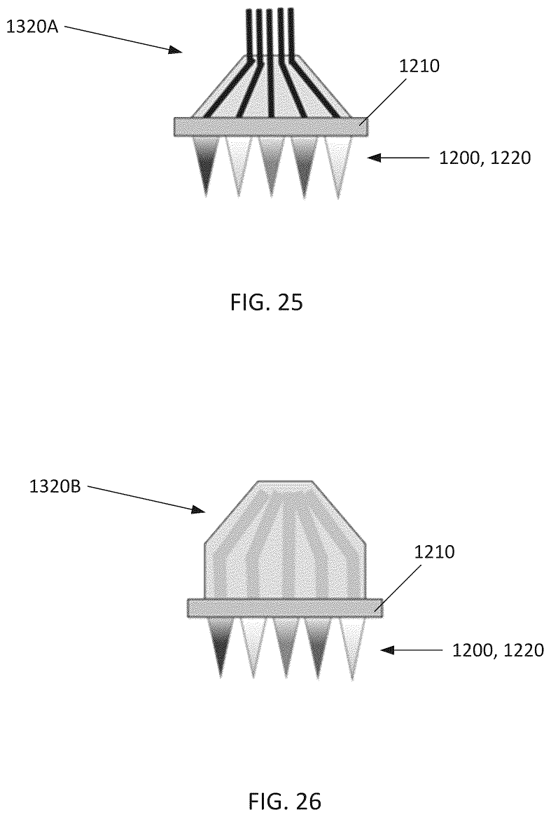

Unlike the snapshot-based approach shown in FIG. 23 where numerous microneedles 1220 are signally coupled to the multiplex sensing system 1000 via respective optical fibers 1230, the embodiments shown in FIGS. 24 through 26 sequentially obtain the different spectra instead of a snapshot of all the spectra. Particularly, in the example of FIGS. 24 through 26, the individual optical microneedles 1220 may be coupled to waveguides 1320, as opposed to optical fibers 1230. In some embodiments, the waveguides 1320 may define a tapered fused fiber waveguide 1320A, shown in FIG. 25, or a semiconductor photonic waveguide 1320B, shown in FIG. 26. Regardless of the type of intermediate connector (e.g., optical fiber 1230 or waveguide 1320), a microelectromechanical system (MEMS) optical switch (or transistor) 1330 is used to control from which individual optical microneedle 1220 data is being transmitted. In this way, the acquired raw data from the various optical microneedles 1220 is then routed (such as through an angled mirror 1340 or other related device) to then pass through one or more collimating lenses 1300, one or more VPGs 1310, one or more focusing lenses 1305 and into the detector 1400. In some embodiments, the MEMS optical switch 1330 forms the same N×M array as that of the optical sensor array 1200. As previously noted, one or a series of lenses 1300, 1305 and the VPG 1310 are used to collect spectra from a single optical microneedle 1220 at a time for subsequent transmission to the detector 1400. Although the front-end components of the system depicted in FIG. 24 may represent a simpler, lower cost, and lower power version of the one shown for the multiplex sensing system 1000 in FIG. 23, they must cycle through each individual optical microneedle 1220 instead of being able to detect output from the entire optical sensor array 1200 simultaneously. Moreover, the discussion herein of hyperspectral imaging is most appropriate for the embodiment depicted in FIG. 23.

Referring next to FIG. 27, as previously noted, spectral imaging may be achieved through a scanning-based approach to acquire the entire dataset over a certain measurement period, as determined by the multiplex sensing system 1000. In some embodiments, this scanning-based approach can be a line scan 1600 that is able to collect data from one row of optical microneedles 1220 (presently shown as four in number but understood to include numbers fewer or greater) at a time using (as with the embodiments of FIGS. 23 through 27) a series of lenses 1300, 1305, one or more VPGs 1310 and detector 1400. This presents an intermediate option between the more comprehensive multiplex sensing system 1000 of FIG. 23 and the simpler multiplex sensing system 1000 of FIGS. 24 through 26, depending on overall system cost versus the ability to monitor multiple analytes at the exact same time. A signal transmission device 1700 is depicted generally to represent any suitable device to transmit the acquired optical data from a particular row of the microneedles 1220 to the collimating lens 1300. In one iteration, it could be the linear fiber array 1240 of FIG. 23 or the waveguide 1320 of FIGS. 24 through 26.

Regardless of the particular embodiment of the multiplex sensing system 1000 that is depicted in FIGS. 23 through 27, medical and related forms of continuous health monitoring of multiple analytes in a minimally invasive manner is enabled. As previously discussed, monitoring dissolved oxygen and dissolved metal ion and chloride levels is useful in the detection of hypoxia, hyperoxia, dehydration and other conditions. In one example, the configuration of FIGS. 23 through 27 have numerous tailored optical microneedles 1200 or related optical sensors, such that detection of dehydration may be extended to assess not just the hydration status but also the type of dehydration through having various analyte-sensing dyes for detection of multiple electrolytes. In this regard, the ability to simultaneously detect multiple analytes is important as relative levels of different analytes provide valuable health information that is otherwise difficult or impossible to acquire without a real-time approach as enabled by the multiplex sensing system 1000 of FIGS. 23 through 27. It will be understood that this is but one example of where a multiplexed-based sensing approach may be achieved through the embodiments of FIGS. 23 through 27. In yet another example, monitoring electrolytes is also beneficially used in extreme environments as a way to provide early indication of hypothermia and hyperthermia, while in yet another low or high levels of various electrolytes can provide indication of a variety of diseases and conditions such as parathyroid hormone deficiency, cardiac arrhythmia, adrenal hyperfunction and kidney disease. In still another example, continuous monitoring of certain electrolyte levels may be achieved for patients with certain conditions, such as cystic fibrosis and epilepsy. In yet another example, sensing of tissue oxygenation could indicate hypoxia or hyperoxia and could be helpful for monitoring patients with sepsis, certain cancers, critical limb ischemia and other conditions.

Also as previously noted, in one form some or all of the components making up the multiplex sensing system 1000 of FIGS. 23 through 27 are configured to be wearable such that it can be used for mobile-based continuous health monitoring without having to have the individual be tethered or otherwise secured to stationary monitoring equipment. In some embodiments, the continuous monitoring may be achieved with a single wearable microneedle array 1200 in the form of a disposable, one-time use wearable patch of microneedles 1220 that in turn allows the acquired data to be conveyed over a wireless version of the network 1500, all as previously discussed.

In light of the foregoing, it will be apparent that light-transmissive microneedle arrays 250 depicted as part of the microneedle assembly 200 in FIGS. 1B and 2 and the microneedle array 1200 of FIGS. 23 through 27 offer minimally-invasive, real-time ways to measure certain analytes of interest within the interstitial fluid, including (as noted with particularity for the embodiments of FIGS. 23 through 27) the ability to sense and detect numerous different analytes. In some embodiments, the microneedle assemblies 200, 1200 operate predominantly or exclusively in the visible part of the electromagnetic spectrum. Regardless of which part of the spectrum the acquired data resides, these measurements may be operated upon by a computer-controlled circuit (such as the computer system 300, also as depicted in FIG. 1A) in order to correlate the measurements to various physiological conditions of interest, such as electrolyte concentrations and tissue oxygenation. This correlation is generally done by comparing the results to known algorithmic or empirical baselines.

Although not shown, it will be appreciated that the multiplex sensing system 1000 may be computer-based in a manner similar to the analyte-sensing system 100 of FIG. 1 that includes a computer system 300. In this way, the various activities related to the detection, analysis and reporting of the results may be performed automatically. As with the analyte-sensing system 100, the multiplex sensing system 1000 with computer system 300 may function as a control circuit that may be used for implementing the various processes described herein, including where the computer system 300 forms a part of a control circuit.

### OTHER CONSIDERATIONS

The phrasing and terminology used herein is for the purpose of description and should not be regarded as limiting.

Measurements, sizes, amounts, and the like may be presented herein in a range format. The description in range format is provided merely for convenience and brevity and should not be construed as an inflexible limitation on the scope of the invention. Accordingly, the description of a range should be considered to have specifically disclosed all the possible subranges as well as individual numerical values within that range. For example, description of a range such as 1-20 meters should be considered to have specifically disclosed subranges such as 1 meter, 2 meters, 1-2 meters, less than 2 meters, 10-11 meters, 10-12 meters, 10-13 meters, 10-14 meters, 11-12 meters, 11-13 meters, etc.

Within the present disclosure, one or more of the following claims may utilize the term “wherein” as a transitional phrase. For the purposes of defining features discussed in the present disclosure, this term is introduced in the claims as an open-ended transitional phrase that is used to introduce a recitation of a series of characteristics of the structure and should be interpreted in like manner as the more commonly used open-ended preamble term “comprising” and its variants that do not preclude the possibility of additional acts or structures.

Within the present disclosure, terms such as “preferably”, “generally” and “typically” are not utilized to limit the scope of the claims or to imply that certain features are critical, essential, or even important to the disclosed structures or functions. Rather, these terms are merely intended to highlight alternative or additional features that may or may not be utilized in a particular embodiment of the disclosed subject matter. Likewise, it is noted that the terms “substantially” and “approximately” and their variants are utilized to represent the inherent degree of uncertainty that may be attributed to any quantitative comparison, value, measurement or other representation. As such, use of these terms represents the degree by which a quantitative representation may vary from a stated reference without resulting in a change in the basic function of the subject matter at issue.

Within the present disclosure, the term “individual”—when referring to someone or something undergoing an analyte-sensing procedure—is meant to include a person or other animate object (such as a dog, cat, other pet, livestock or the like that may benefit from the microneedle-based sensing of analytes in fluids as discussed herein). Accordingly, the various terms used herein to identify such individual, including “person”, “user”, “animal” or “patient” are deemed to be equivalents within the present disclosure, and that any greater degree of specificity of such terms will be apparent from the context.

Within the present disclosure, the use of the prepositional phrase “at least one of” is deemed to be an open-ended expression that has both conjunctive and disjunctive attributes. For example, a claim that states “at least one of A, B and C” (where A, B and C are definite or indefinite articles that are the referents of the prepositional phrase) means A alone, B alone, C alone, A and B together, A and C together, B and C together or A, B and C together.

As used in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

The use of “including,” “comprising,” “having,” “containing,” “involving,” and variations thereof, is meant to encompass the items listed thereafter and additional items.

Use of ordinal terms such as “first,” “second,” “third,” etc., in the claims to modify a claim element does not by itself connote any priority, precedence, or order of one claim element over another or the temporal order in which acts of a method are performed. Ordinal terms are used merely as labels to distinguish one claim element having a certain name from another element having a same name (but for use of the ordinal term), to distinguish the claim elements.

Within the present disclosure, claimed elements that are adapted or configured to accomplish a specified objective are those that are designed and constructed to be used as a particular device in a particular way to have a particular structural attribute in order to accomplish such objective using positive, present-tense and concrete actions rather than merely as being capable of achieving such objective. This distinction is particularly apparent when such element is described or shown herein as being in an actual state of configuration or adaptation.

Within the present disclosure, the following claims are not intended to be interpreted based on 35 USC 112 (f) unless and until such claim limitations expressly use the phrase “means for” or “steps for” followed by a statement of function void of further structure. Moreover, the corresponding structures, materials, acts and equivalents of all means or step plus function elements in the claims that follow are intended to include any structure, material or act for performing the function in combination with other claimed elements as specifically claimed.

Within the present disclosure, the singular forms “a,” “an” and “the” include plural references unless the context clearly dictates otherwise. The modifier “about” used in connection with a quantity is inclusive of the stated value and has the meaning dictated by the context (for example, it includes at least the degree of error associated with the measurement of the particular quantity). The modifier “about” should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%” may indicate a range of 9% to 11%, and “about 1” may mean from 0.9 to 1.1. Other meanings of “about” may be apparent from the context, such as rounding off, so, for example “about 1” may also mean from 0.5 to 1.4.

The term “approximately”, the phrase “approximately equal to”, and other similar phrases, as used in the specification and the claims (e.g., “X has a value of approximately Y” or “X is approximately equal to Y”), should be understood to mean that one value (X) is within a predetermined range of another value (Y). The predetermined range may be plus or minus 20%, 10%, 5%, 3%, 1%, 0.1%, or less than 0.1%, unless otherwise indicated.

For the recitation of numeric ranges herein, each intervening number there between with the same degree of precision is explicitly contemplated. For example, for the range of 6 to 9, the numbers 7 and 8 are contemplated in addition to 6 and 9, and for the range 6.0 to 7.0, the number 6.0, 6.1, 6.2, 6.3, 6.4, 6.5, 6.6, 6.7, 6.8, 6.9 and 7.0 are explicitly contemplated.

The present description is for purpose of illustration and is not intended to be exhaustive or limited. Many modifications and variations will be apparent to those of ordinary skill in the art without departing from the scope and spirit of the present disclosure. Aspects of the present disclosure were chosen and described in order to best explain the principles and practical applications, and to enable others of ordinary skill in the art to understand the subject matter contained herein for various embodiments with various modifications as are suited to the particular use contemplated.

Unless otherwise defined, all technical and scientific terms used herein that relate to materials and their processing have the same meaning as commonly understood by one of ordinary skill in the art. In case of conflict, the present document, including definitions, will control.

Furthermore, connections between components or systems within the figures are not intended to be limited to direct connections. Rather, data or signals between these components may be modified, re-formatted, or otherwise changed by intermediary components. Also, additional or fewer connections may be used. The terms “coupled,” “connected,” or “communicatively coupled” shall be understood to include direct connections, indirect connections through one or more intermediary devices, wireless connections, and so forth.

Reference in the specification to “one embodiment,” “preferred embodiment,” “an embodiment,” “some embodiments,” or “embodiments” means that a particular feature, structure, characteristic, or function described in connection with the embodiment is included in at least one embodiment of the invention and may be in more than one embodiment. Also, the appearance of the above-noted phrases in various places in the specification is not necessarily referring to the same embodiment or embodiments.

The use of certain terms in various places in the specification is for illustration purposes only and should not be construed as limiting. A service, function, or resource is not limited to a single service, function, or resource; usage of these terms may refer to a grouping of related services, functions, or resources, which may be distributed or aggregated.

Furthermore, one skilled in the art shall recognize that: (1) certain steps may optionally be performed; (2) steps may not be limited to the specific order set forth herein; (3) certain steps may be performed in different orders; and (4) certain steps may be performed simultaneously or concurrently.

Implementations of the subject matter and the operations described in this specification can be implemented in digital electronic circuitry, or in computer software, firmware, or hardware, including the structures disclosed in this specification and their structural equivalents, or in combinations of one or more of them. Implementations of the subject matter described in this specification can be implemented as one or more computer programs, i.e., one or more modules of computer program instructions, encoded on computer storage medium for execution by, or to control the operation of, data processing apparatus. Alternatively or in addition, the program instructions can be encoded on an artificially generated propagated signal, e.g., a machine-generated electrical, optical, or electromagnetic signal, that is generated to encode information for transmission to suitable receiver apparatus for execution by a data processing apparatus. A computer storage medium can be, or be included in, a computer-readable storage device, a computer-readable storage substrate, a random or serial access memory array or device, or a combination of one or more of them. Moreover, while a computer storage medium is not a propagated signal, a computer storage medium can be a source or destination of computer program instructions encoded in an artificially-generated propagated signal. The computer storage medium can also be, or be included in, one or more separate physical components or media (e.g., multiple CDs, disks, or other storage devices).

The operations described in this specification can be implemented as operations performed by a data processing apparatus on data stored on one or more computer-readable storage devices or received from other sources.

The term “data processing apparatus” encompasses all kinds of apparatus, devices, and machines for processing data, including by way of example a programmable processor, a computer, a system on a chip, or multiple ones, or combinations, of the foregoing. The apparatus can include special purpose logic circuitry, e.g., an FPGA (field programmable gate array) or an ASIC (application-specific integrated circuit). The apparatus can also include, in addition to hardware, code that creates an execution environment for the computer program in question, e.g., code that constitutes processor firmware, a protocol stack, a database management system, an operating system, a cross-platform runtime environment, a virtual machine, or a combination of one or more of them. The apparatus and execution environment can realize various different computing model infrastructures, such as web services, distributed computing and grid computing infrastructures.

A computer program (also known as a program, software, software application, script, or code) can be written in any form of programming language, including compiled or interpreted languages, declarative or procedural languages, and it can be deployed in any form, including as a stand-alone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub-programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform actions by operating on input data and generating output. The processes and logic flows can also be performed by, and apparatus can also be implemented as, special purpose logic circuitry, e.g., an FPGA (field programmable gate array) or an ASIC (application-specific integrated circuit).

Processors suitable for the execution of a computer program include, by way of example, both general and special purpose microprocessors, and any one or more processors of any kind of digital computer. Generally, a processor will receive instructions and data from a read-only memory or a random access memory or both. The essential elements of a computer are a processor for performing actions in accordance with instructions and one or more memory devices for storing instructions and data. Generally, a computer will also include, or be operatively coupled to receive data from or transfer data to, or both, one or more mass storage devices for storing data, e.g., magnetic disks, magneto-optical disks, optical disks, or solid state drives. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device (e.g., a universal serial bus (USB) flash drive), to name just a few. Devices suitable for storing computer program instructions and data include all forms of non-volatile memory, media and memory devices, including, by way of example, semiconductor memory devices, e.g., EPROM, EEPROM, and flash memory devices; magnetic disks, e.g., internal hard disks or removable disks; magneto-optical disks; and CD-ROM and DVD-ROM disks. The processor and the memory can be supplemented by, or incorporated in, special purpose logic circuitry.

To provide for interaction with a user, implementations of the subject matter described in this specification can be implemented on a computer having a display device, e.g., a CRT (cathode ray tube) or LCD (liquid crystal display) monitor, for displaying information to the user and a keyboard and a pointing device, e.g., a mouse, a trackball, a touchpad, or a stylus, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's client device in response to requests received from the web browser.

Implementations of the subject matter described in this specification can be implemented in a computing system that includes a back-end component, e.g., as a data server, or that includes a middleware component, e.g., an application server, or that includes a front-end component, e.g., a client computer having a graphical user interface or a Web browser through which a user can interact with an implementation of the subject matter described in this specification, or any combination of one or more such back-end, middleware, or front-end components. The components of the system can be interconnected by any form or medium of digital data communication, e.g., a communication network. Examples of communication networks include a local area network (“LAN”) and a wide area network (“WAN”), an inter-network (e.g., the Internet), and peer-to-peer networks (e.g., ad hoc peer-to-peer networks).

The computing system can include clients and servers. A client and server are generally remote from each other and typically interact through a communication network. The relationship of client and server arises by virtue of computer programs running on the respective computers and having a client-server relationship to each other. In some implementations, a server transmits data (e.g., an HTML page) to a client device (e.g., for purposes of displaying data to and receiving user input from a user interacting with the client device). Data generated at the client device (e.g., a result of the user interaction) can be received from the client device at the server.

In some embodiments, aspects of the systems and methods described herein may be implemented using ML and/or AI technologies.

“Machine learning” generally refers to the application of certain techniques (e.g., pattern recognition and/or statistical inference techniques) by computer systems to perform specific tasks. Machine learning techniques may be used to build models based on sample data (e.g., “training data”) and to validate the models using validation data (e.g., “testing data”). The sample and validation data may be organized as sets of records (e.g., “observations” or “data samples”), with each record indicating values of specified data fields (e.g., “independent variables,” “inputs,” “features,” or “predictors”) and corresponding values of other data fields (e.g., “dependent variables,” “outputs,” or “targets”). Machine learning techniques may be used to train models to infer the values of the outputs based on the values of the inputs. When presented with other data (e.g., “inference data”) similar to or related to the sample data, such models may accurately infer the unknown values of the targets of the inference data set.

As used herein, “model” may refer to any suitable model artifact generated by the process of using a machine learning algorithm to fit a model to a specific training data set. The terms “model,” “data analytics model,” “machine learning model” and “machine learned model” are used interchangeably herein.

As used herein, the “development” of a machine learning model may refer to construction of the machine learning model. Machine learning models may be constructed by computers using training data sets. Thus, “development” of a machine learning model may include the training of the machine learning model using a training data set. In some cases (generally referred to as “supervised learning”), a training data set used to train a machine learning model can include known outcomes (e.g., labels or target values) for individual data samples in the training data set. For example, when training a supervised computer vision model to detect images of cats, a target value for a data sample in the training data set may indicate whether or not the data sample includes an image of a cat. In other cases (generally referred to as “unsupervised learning”), a training data set does not include known outcomes for individual data samples in the training data set.

Following development, a machine learning model may be used to generate inferences with respect to “inference” data sets. For example, following development, a computer vision model may be configured to distinguish data samples including images of cats from data samples that do not include images of cats. As used herein, the “deployment” of a machine learning model may refer to the use of a developed machine learning model to generate inferences about data other than the training data.

Each numerical value presented herein, for example, in a table, a chart, or a graph, is contemplated to represent a minimum value or a maximum value in a range for a corresponding parameter. Accordingly, when added to the claims, the numerical value provides express support for claiming the range, which may lie above or below the numerical value, in accordance with the teachings herein. Absent inclusion in the claims, each numerical value presented herein is not to be considered limiting in any regard.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In certain implementations, multitasking and parallel processing may be advantageous. Other steps or stages may be provided, or steps or stages may be eliminated, from the described processes. Accordingly, other implementations are within the scope of the following claims.

It will be appreciated by those skilled in the art that the preceding examples and embodiments are exemplary and not limiting to the scope of the present disclosure. It is intended that all permutations, enhancements, equivalents, combinations, and improvements thereto that are apparent to those skilled in the art upon a reading of the specification and a study of the drawings are included within the true spirit and scope of the present disclosure. It shall also be noted that elements of any claims may be arranged differently including having multiple dependencies, configurations, and combinations.

While particular embodiments have been illustrated and described herein, it should be understood that various other changes and modifications may be made without departing from the spirit and scope of the claimed subject matter. Moreover, although various aspects of the claimed subject matter have been described herein, such aspects need not be utilized in combination. It is therefore intended that the appended claims cover all such changes and modifications that are within the scope of the claimed subject matter.

What is claimed is:

1. An optical processing system comprising:
a plurality of lenses configured to receive individual light signals from analyte-sensing chromophores disposed on an array when the analyte-sensing chromophores are illuminated by light while being exposed to different specific analytes within an interstitial fluid;
at least one volume phase grating (VPG) communicatively coupled to at least one of the plurality of lenses; and
a detector communicatively coupled to the at least one VPG so that light signals from the analyte-sensing chromophores are focused by the plurality of lenses to the at least one VPG and transmitted to the detector, wherein the detector is operable to generate spectra with each spectrum corresponding to an individual light signal from a single analyte-sensing chromophore.

2. The optical processing system of claim 1, further comprising a coupling device disposed upstream of the array that contains the analyte-sensing chromophores, the coupling device operable to convey light signals from the analyte-sensing chromophores to the plurality of lenses.

3. The optical processing system of claim 2, wherein the coupling device comprises a linear fiber array, a non-linear fiber array, or a waveguide.

4. The optical processing system of claim 1, wherein the plurality of lenses comprise:
a collimating lens disposed in an optical path between the coupling device and the VPG; and
a focusing lens disposed in an optical path between the VPG and the detector.

5. The optical processing system of claim 1, wherein each spectrum registers on the detector as a data cube.

6. The optical processing system of claim 1, wherein the array is an array of microneedles with the analyte-sensing chromophores disposed therein, and wherein sensing occurs in the interstitial fluid located between tissue spaces in human skin.

7. The optical processing system of claim 6, wherein the array of microneedles comprises microneedles arranged in N number of rows and M number of columns.

8. The optical processing system of claim 6, wherein the individual light signals from the analyte-sensing chromophores are obtained by scanning the array of microneedles or by sequentially using a microelectromechanical system (MEMS) optical switch or a transistor.

9. The optical processing system of claim 1, wherein each spectrum is an indicia of a specific analyte present in the interstitial fluid.

10. A multiplex sensing system comprising:
an optical sensor array with microneedle-chromophore combinations, wherein the microneedle-chromophore combinations comprise analyte-sensing chromophores whose at least one photophysical property is altered and optically detected when the analyte-sensing chromophores are illuminated with light while exposed to a specific analyte in an interstitial fluid;
a plurality of lenses;
a coupling device disposed between the optical sensor array and the plurality of lenses, the coupling device operable to receive and convey optical signals from the microneedle-chromophore combinations to the plurality of lenses;
a volume phase grating (VPG) operatively coupled to the plurality of lenses, the VPG operable to aid with a formation of spectra for the received optical signals; and
a detector communicatively coupled to the VPG via one or more additional lenses, wherein the detector is operable to:
detect the optical signals from the analyte-sensing chromophores when the analyte-sensing chromophores are exposed to at least a portion of the specific analyte within the interstitial fluid and after the analyte-sensing chromophores are illuminated with light; and
generate a spectrum for each optical signal detected, the generated spectrum is an indicia of a specific analyte being present in the interstitial fluid.

11. The multiplex sensing system of claim 10, wherein the light used to illuminate the analyte-sensing chromophores when exposed to the specific analytes is provided by at least one of the multiplex sensing system and a computer system controlling the multiplex sensing system.

12. The multiplex sensing system of claim 10, wherein the optical sensor array and the coupling device define a modular assembly that forms a quick-connect coupling with at least one other component of the multiplex sensing system.

13. The multiplex sensing system of claim 12, wherein the modular assembly forms a quick-connect coupling with at least one lens from the plurality of lenses.

14. The multiplex sensing system of claim 10, wherein the coupling device comprises a linear fiber array, a non-linear fiber array, or a waveguide.

15. The multiplex sensing system of claim 14, wherein the waveguide comprises a semiconductor photonic waveguide or a fused fiber waveguide.

16. The multiplex sensing system of claim 10, wherein at least one of the plurality of lenses, the coupling device, the VPG, the one or more additional lenses, and the detector define an optical processing system.

17. The multiplex sensing system of claim 16, wherein at least a portion of the optical processing system defines a wearable form factor.

18. The multiplex sensing system of claim 10, wherein the plurality of lenses comprises a collimating lens disposed in an optical path between the coupling device and the VPG, and the one or more additional lenses comprise a focusing lens disposed in an optical path between the VPG and the detector.

19. The multiplex sensing system of claim 10, wherein an agent comprising a biocompatible polymer and the analyte-sensing chromophore is engineered to interact with the specific analyte, the agent further comprising a non analyte-sensing reference chromophore.

20. The multiplex sensing system of claim 10, wherein the detector is capable of concurrently detecting the optical signals from all the analyte-sensing chromophores and concurrently generating spectra corresponding to the detected optical signals.
